(19) United States
(12) Patent Application Publication
(10) Pub. No.: US 2008/0146811 A1
(43) Pub. Date: Jun. 19, 2008

(54) Compounds and Methods For Treatment of Thrombosis

(76) Inventors: Hongfeng Deng, Acton, MA (US); Jian Lin, Walpole, MA (US); Zihong Guo, Southbury, CT (US); Harold V. Meyers, Weston, MA (US); Sherin S. Abdel-Meguid, Exton, PA (US); Robert E. Babine, Carlsbad, CA (US)

Correspondence Address: CLARK & ELBING LLP, 101 FEDERAL STREET, BOSTON, MA, 02110, US

(21) Appl. No.: 11/658,121

(22) PCT Filed: Jul. 22, 2005
(86) PCT No.: PCT/US2005/026022

§ 371 Date: Jul. 11, 2007

Related U.S. Application Data
(60) Provisional application No. 60/590,718, filed on Jul. 23, 2004.

(51) Int. Cl. C07D 401/12; C07D 209/14

(52) U.S. Cl. 546/277.4; 548/495

(57) ABSTRACT

The invention provides compounds, pharmaceutical compositions, and methods for the treatment of thromboembolic disorders, such as, for example, arterial cardiovascular thromboembolic disorders, venous cardiovascular thromboembolic disorders, or thromboembolic disorders in the chambers of the heart.

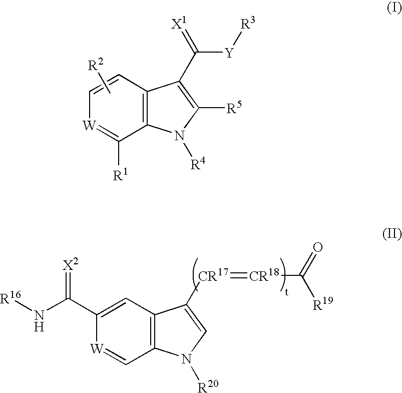

### BACKGROUND OF THE INVENTION

Blood coagulation is the first line of defense against blood loss following injury. The blood coagulation “cascade” involves a number of circulating serine protease zymogens, regulatory cofactors and inhibitors, as shown in FIG. 1. Each enzyme, once generated from its zymogen, specifically cleaves the next zymogen in the cascade to produce an active protease. This process is repeated until finally thrombin cleaves the fibrinopeptides from fibrinogen to produce fibrin that polymerizes to form a blood clot. Although efficient clotting limits the loss of blood at a site of trauma, it also poses the risk of systemic coagulation resulting in massive thrombosis. Under normal circumstances, hemostasis maintains a balance between clot formation (coagulation) and clot dissolution (fibrinolysis). However, in certain disease states such as acute myocardial infarction and unstable angina, the rupture of an established atherosclerotic plaque results in abnormal thrombus formation in the coronary arterial vasculature.

Despite the availability of a number of approved anticoagulant therapies, myocardial infarction, unstable angina, atrial fibrillation, stroke, pulmonary embolism, and deep vein thrombosis represent areas of major medical need. Cardiovascular diseases (e.g., acute myocardial infarction, stroke, and pulmonary embolism) disable or kill more people in the developed world than any other disease. Over two million patients are hospitalized each year in the U.S. for acute arterial thrombosis and stroke. The worldwide population for acute arterial antithrombotic therapy is five to six million, while over 25 million patients have chronic arterial thrombosis. Over 10 million individuals are candidates for venous thrombosis therapy.

A large medical need exists for novel anticoagulation drugs that lack some or all of the side effects of currently available drugs, such as the risk of bleeding episodes and patient-to-patient variability that results in the need for close monitoring and titration of therapeutic doses. Current anticoagulant therapies that dominate the market include injectable unfractionated and low molecular weight (LMW) heparin, and orally administered warfarin (coumadin).

Three phases of the coagulation cascade can be described, namely initiation, amplification, and propagation (see FIG. 1). Inhibiting enzymes of the propagation phase, i.e., Factor Xa and Factor IIa (thrombin), has been an area of active interest in the pharmaceutical industry for some time. The first generation of thrombin inhibitors to reach the clinic were polypeptides derived from natural sources, such as the potent anticoagulant, hirudin, which is a leech peptide. Potent, orally available, small molecule thrombin inhibitors have been discovered over the past few decades. Some of these are now in the clinic or are ready to be marketed. Efforts to develop potent Factor Xa inhibitors are not far behind. Targeting enzymes involved in propagation (e.g., hirudin) does not appear to be ideal since inhibitors of this phase of the coagulation cascade are associated with severe bleeding. This is further supported by findings that Factor V and Factor X deficiencies are associated with severe bleeding episodes.

Several new treatments under development are aimed at the initiation phase that involves Factor VII and tissue factor (TF). These include an active site-blocked Factor VIIa, a high affinity neutralizing antibody against TF, and a nematode protein (NAPcc) that inhibits Factor VIIa/TF. Because these approaches target the very start of the coagulation cascade, they may lead to bleeding episodes.

Due to the limited efficacy and adverse side-effects of some current therapeutics for the inhibition of undesirable thrombosis (e.g., deep vein thrombosis and stroke), improved compounds and methods are needed for preventing or treating undesirable thrombosis.

### SUMMARY OF THE INVENTION

Accordingly, in a first aspect, the invention features a compound of formula (I):

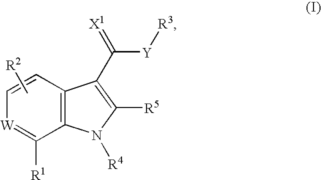

or a derivative, isomer, pharmaceutically acceptable salt, pharmaceutically acceptable solvate, pharmaceutically active metabolite, or prodrug thereof, where

W is N or CR⁶, where R⁶ is H, halo, hydroxy, substituted or unsubstituted C₁₋₆ alkyl, substituted or unsubstituted C₂₋₆ alkenyl, substituted or unsubstituted C₁₋₆ alkoxy, substituted or unsubstituted C₂₋₁₂ alkoxyalkyl, where the alkylene group is of one to six carbon atoms, substituted or unsubstituted C₆ or C₁₀ aryl, substituted or unsubstituted C₆ or C₁₀ aryloxy, or —(CH₂)_{q}NR^{G6}R^{H6}, where q is an integer of from zero to two and each of R^{G6} and R^{H6} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N6}, where R^{N6} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A6}, where R^{A6} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) —CONR^{B6}R^{C6}, where each of R^{B6} and R^{C6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, and substituted or unsubstituted C₂₋₁₅ heterocyclylalkyl, where the alkylene group is of one to six carbon atoms, or R^{B6} taken together with R^{C6} and N forms a substituted or unsubstituted 5- or 6-membered ring, (o) —S(O)₂R^{S6}, where R^{S1} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (p) a peptide chain of 1-4 natural or unnatural alpha-amino acid residues, where the peptide is linked via its C-terminal carboxy group to N, with the proviso that no two groups are bound to the nitrogen atom through a carbonyl group or a sulfonyl group;

X¹ is (H,H) or NR⁷, where R⁷ is H, C₁₋₆ alkyl, OH, NH₂, NO₂, CO₂R^{7a}, where R^{7a} is C₁₋₆ alkyl, or R⁷ taken together with R³ forms a 5- or 6-membered ring via a C₁ or C₂ linkage;

Y is NH or O, provided that when Y is O, X¹ is (H,H);

R¹ is a substituted or unsubstituted C₂₋₇ alkanoyl, substituted or unsubstituted C₁₋₆ alkyl, substituted or unsubstituted C₂₋₆ alkenyl, substituted or unsubstituted C₁₋₆ alkoxy, substituted or unsubstituted C₂₋₁₂ alkoxyalkyl, where the alkylene group is of one to six carbon atoms, substituted or unsubstituted C₁₋₆ alkylsulfinyl, substituted or unsubstituted C₂₋₁₂ alkylsulfinylalkyl, where the alkylene group is of one to six carbon atoms, substituted or unsubstituted C₁₋₆ alkylsulfonyl, substituted or unsubstituted C₆ or C₁₀ aryl, substituted or unsubstituted C₇₋₁₆ arylalkyl, where the alkylene group is of one to six carbon atoms, C₁₋₆ aminoalkyl, substituted or unsubstituted C₇ or C₁₁ aryloyl, substituted or unsubstituted C₃₋₈ cycloalkyl, substituted or unsubstituted C₄₋₁₄ cycloalkylalkyl, where the alkylene group is of one to six carbon atoms, substituted or unsubstituted C₁₋₉ heterocyclyl, substituted or unsubstituted C₁₋₉ (heterocyclyl)oxy, substituted or unsubstituted C₂₋₁₀ (heterocyclyl)oyl, substituted or unsubstituted C₁₆ thioalkoxy, substituted or unsubstituted C₂₋₁₂ thioalkoxyalkyl, where the alkylene group is of one to six carbon atoms, substituted or unsubstituted C₆ or C₁₀ aryloxy, substituted or unsubstituted C₃₋₈ cycloalkoxy, substituted or unsubstituted C₄₋₁₄ cycloalkylalkoxy, substituted or unsubstituted C₇₋₁₆ arylalkoxy, amidino, guanidino, ureido, —(CH₂)_{q}CO₂R^{A1}, where q is an integer of from zero to four and R^{A1} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}COR^{K1}, where q is an integer of from zero to four and R^{K1} is a peptide chain of 1-4 natural or unnatural alpha-amino acid residues, where the peptide is linked via its N-terminal amine to the CO group, —(CH₂)_{q}COR^{N1}, where q is an integer of from zero to four and R^{N1} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CONR^{B1}R^{C1}, where q is an integer of from zero to four and each of R^{B1} and R^{C1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B1} taken together with R^{C1} and N forms a substituted or unsubstituted 5- or 6-membered ring, —(CH₂)_{q}S(O)₂R^{D1}, where q is an integer of from zero to four and R^{D1} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide of 1-4 natural or unnatural alpha-amino acid residues, where the peptide is linked via its N-terminal amine to the S(O)₂ group, —(CH₂)_{q}S(O)₂NR^{E1}R^{F1}, where q is an integer of from zero to four and each of R^{E1} and R^{F1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E1} taken together with R^{F1} and N forms a substituted or unsubstituted 5- or 6-membered ring, —(CH₂)_{q}CR^{U1}═CR^{V1}CO₂R^{A1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{A1} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CR^{U1}=CR^{V1}COR^{K1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{K1} is a peptide chain of 1-4 natural or unnatural alpha-amino acid residues, where the peptide is linked via its N-terminal amine to the CO group, —(CH₂)_{q}CR^{U1}═CR^{V1}COR^{N1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{N1} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CR^{U1}═CR^{V1}CONR^{B1}R^{C1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and each of R^{B1} and R^{C1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B1} taken together with R^{C1} and N forms a substituted or unsubstituted 5- or 6-membered ring, —(CH₂)_{q}CR^{U1}═CR^{V1}S(O)₂R^{D1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{D1} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, —(CH₂)_{q}CR^{U1}═CR^{V1}S(O)₂NR^{E1}R^{F1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₁₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and each of R^{E1} and R^{F1} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E1} taken together with R^{F1} and N forms a substituted or unsubstituted 5- or 6-membered ring, —(CH₂)_{q}NR^{G1}R^{H1}, where q is an integer of from zero to four and each of R^{G1} and R^{H1} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N1}, where R^{N1} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A1}, where R^{A1} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) —S(O)₂R⁵¹, where R^{S1} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (o) a peptide chain of 1-4 natural or unnatural alpha-amino acid residues, where the peptide is linked via its C-terminal carboxy group to N, with the proviso that no two groups are bound to the nitrogen atom through a carbonyl group or a sulfonyl group, O(CH₂)_{q}OR^{A1}, where q is an integer of from 0 to 4 and R^{A1} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or —(CH₂)_{q}NR^{b1}CONR^{E1}R^{F1}, where q is an integer of from 0 to 4, R^{b1} is hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and each of R^{E1} and R^{F1} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E1} taken together with R^{F1} and N forms a substituted or unsubstituted 5- or 6-membered ring;

R² is an H or a substituted or unsubstituted C₁₋₆ alkyl, substituted or unsubstituted C₂₋₆ alkenyl, hydroxyl, substituted or unsubstituted C₁₋₆ alkoxy, substituted or unsubstituted C₇₋₁₆ aralkoxy, trifluoromethyl, halo, amidino, N-hydroxyamidino, guanidino, —(CH₂)_{q}CO₂R^{A2}, where q is an integer of from zero to two and R^{A2} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CONR^{B2}R^{C2}, where q is an integer of from zero to two and each of R^{B2} and R^{C2} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) Substituted or unsubstituted C₁₋₉ heterocyclyl, and (f) substituted or unsubstituted C₂₋₁₅ heterocyclylalkyl, where the alkylene group is of one to six carbon atoms, or R^{B2} taken together with R^{C2} and N forms a substituted or unsubstituted 5- or 6-membered ring, —(CH₂)_{q}S(O)₂R^{D2}, where q is an integer of from zero to two and R^{D2} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}S(O)₂NR^{E2}R^{F2}, where q is an integer of from zero to two and each of R^{E2} and R^{F2} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E2} taken together with R^{F2} and N forms a substituted or unsubstituted 5- or 6-membered ring, or —(CH₂)_{q}NR^{G2}R^{H2}, where q is an integer of from zero to two and each of R^{G2} and R^{H2} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (h) substituted or unsubstituted C₁₋₉ heterocyclyl, (k) substituted or unsubstituted C₂₋₁₅ heterocyclylalkyl, where the alkylene group is of one to six carbon atoms, (l) —COR^{N2}, where R^{N2} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A2}, where R^{A2} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (n) —S(O)₂R^{S2}, where R^{S2} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, or R^{G2} taken together with R^{H2} and N forms a substituted or unsubstituted 5- or 6-membered ring, with the proviso that no two groups are bound to the nitrogen atom through a carbonyl group or a sulfonyl group;

R³ is H or C₁₋₆ alkyl, or when taken together with R⁵ or R⁷ forms a 5- or 6-membered ring via a C₁ or C₂ linkage;

R⁴ is H or a substituted or unsubstituted C₂₋₇ alkanoyl, substituted or unsubstituted C₁₋₆ alkyl, substituted or unsubstituted C₂₋₆ alkenyl, substituted or unsubstituted C₂₋₁₂ alkoxyalkyl, substituted or unsubstituted C₂₋₁₂ alkylsulfinylalkyl, substituted or unsubstituted C₁₋₆ alkylsulfonyl, substituted or unsubstituted C₆ or C₁₀ aryl, substituted or unsubstituted C₇₋₁₆ arylalkyl, substituted or unsubstituted C₁₋₆ aminoalkyl, substituted or unsubstituted C₇ or C₁₁ aryloyl, C₁₋₆ azidoalkyl, carboxaldehyde, carboxamide, C₃₋₈ cycloalkyl, C₄₋₁₄ cycloalkylalkyl, substituted or unsubstituted C₁₋₉ heterocyclyl, C₂₋₁₅ heterocyclylalkyl, C₂₋₁₀ (heterocyclyl)oyl, hydroxy, C₁₋₆ hydroxyalkyl, N-protected aminoalkyl, C₂₋₁₂ thioalkoxyalkyl, C₁₋₄ perfluoroalkyl, substituted or unsubstituted C₆ or C₁₀ aryloxy, —(CH₂)_{q}CR^{U4}═CR^{V4}CO₂R^{A4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{A4} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CR^{U4}═CR^{V4}COR^{K4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{K4} is a peptide chain of 1-4 natural or unnatural alpha-amino acid residues, where the peptide is linked via its N-terminal amine to the CO group, —(CH₂)_{q}CR^{U4}═CR^{V4}COR^{N4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{N4} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CR^{U4}═CR^{V4}CONR^{B4}R^{C4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and each of R^{B4} and R^{C4} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B4} taken together with R^{C4} and N forms a substituted or unsubstituted 5- or 6-membered ring, —(CH₂)_{q}CR^{U4}═CR^{V4}S(O)₂R^{D4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{D4} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, —(CH₂)_{q}CR^{U4}═CR^{V4}S(O)₂NR^{E4}R^{F4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and each of R^{E4} and R^{F4} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E4} taken together with R^{F4} and N forms a substituted or unsubstituted 5- or 6-membered ring, —(CH₂)_{q}(CR^{Y4}R^{Z4})ᵣ(CH₂)ₛCO₂R^{A4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆ alkylsulfonyl, or (l) aminosulfonyl, and R^{A4} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or taken together with R^{Y4} or R^{Z4} forms an optionally substituted or unsubstituted 5- or 6-membered ring, —(CH₂)_{q}(CR^{Y4}R^{Z4})ᵣ(CH₂)ₛC(O)R^{K4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆ alkylsulfonyl, or (l) aminosulfonyl, and R^{K4} is a peptide chain of 1-4 natural or unnatural alpha-amino acid residues, where the peptide is linked via its N-terminal amine to the CO group, —(CH₂)_{q}(CR^{Y4}R^{Z4})ᵣ(CH₂)ₛC(O)R^{N4}, where each of q and s is, independently, an integer of from zero to four, and r is an integer of from zero to one, each of R^{Y4} and R^{Z4} is, independently, (a) hydrogen, (I) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀heteroaroyl, (i) substituted or unsubstituted C₂₋₇ alkoxycarbonyl, (j) aminocarbonyl, (k) substituted or unsubstituted C₁₋₆₇ alkylsulfonyl, or (l) aminosulfonyl, and R^{N4} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, or taken together with R^{Y4} or R^{Z4} forms an optionally substituted or unsubstituted 5- to 7-membered ring, —(CH₂)_{q}(CR^{Y4}R^{Z4})ᵣ(CH₂)ₛCONR^{B4}R^{C4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆ alkylsulfonyl, or (l) aminosulfonyl, and each of R^{B4} and R^{C4} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B4} taken together with R^{Y4} or R^{Z4} forms an optionally substituted or unsubstituted 5- or 6-membered ring, or R^{B4} taken together with R^{C4} and N forms an optionally substituted 5- or 6-membered ring, —(CH₂)_{q}(CR^{Y4}R^{Z4})ᵣ(CH₂)ₛS(O)₂R^{D4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or Cu, aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆₇ alkylsulfonyl, or (l) aminosulfonyl, and R^{D4} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, or taken together with R^{Y4} or R^{Z4} forms an optionally substituted or unsubstituted 5- to 7-membered ring, —(CH₂)_{q}(CR^{Y4}R^{Z4})ᵣ(CH₂)ₛS(O)₂NR^{E4}R^{F4}, where each of q and is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, G) aminocarbonyl, (k) substituted or unsubstituted C₁₋₆₇ alkylsulfonyl, or (l) aminosulfonyl, and each of R^{E4} and R^{F4} is independently selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E4} taken together with R^{Y4} or R^{Z4} forms an optionally substituted or unsubstituted 5- or 6-membered ring, or R^{E4} taken together with R^{F4} forms an optionally substituted 5- or 6-membered ring, —(CH₂)_{q}(CR^{Y4}R^{Z4})ᵣ(CH₂)ₛNR^{G4}R^{H4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆ alkylsulfonyl, or (l) aminosulfonyl, and each of R^{G4} and R^{H4} is independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N4}, where R^{N4} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A4}, where R^{A4} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) —S(O)₂R^{S4}, where R^{S4} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, with the proviso that no two groups are bound to the nitrogen atom through a carbonyl group or a sulfonyl group, or R^{G4} taken together with R^{Y4} or R^{Z4} forms an optionally substituted or unsubstituted 5- to 7-membered ring, or R^{G4} taken together with R^{H4} and N forms an optionally substituted 5- or 6-membered ring, or (o) —CONR^{E4}R^{F4}, and each of R^{E4} and R^{F4} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E4} taken together with R^{Y4} or R^{Z4} forms an optionally substituted or unsubstituted 5- to 7-membered ring, or R^{E4} taken together with R^{F4} and N forms a substituted or unsubstituted 5- or 6-membered ring; and

R⁵ is H or a substituted or unsubstituted C₁₋₆ alkyl, C₂₋₆ alkenyl, C₁₋₆ alkoxy, carboxamide, C₃₋₈ cycloalkyl, hydroxy, nitro, nitrile, C₁₋₆ thioalkoxy, C₁₋₄ perfluoroalkyl, C₁₋₄ perfluoroalkoxy, —(CH₂)_{q}NR^{G5}R^{H5}, where q is zero to two and each of R^{G5} and R^{H5} is, independently, selected from the group consisting of (a) hydrogen, (b) an N-protecting group, (c) alkyl of one to six carbon atoms, (d) alkenyl of two to six carbon atoms, (e) alkynyl of two to six carbon atoms, and (f) cycloalkyl of three to eight carbon atoms, and (g) cycloalkylalkyl, where the cycloalkyl group is of three to eight carbon atoms.

In one embodiment, R¹ is a substituted or unsubstituted C₂₋₇ alkanoyl, substituted or unsubstituted C₇ or C₁₁ aryloyl, substituted or unsubstituted C₂₋₁₀ (heterocyclyl)oyl, substituted or unsubstituted C₁₋₆ alkyl, substituted or unsubstituted C₂₋₆ alkenyl, or —(CH₂)_{q}CR^{U1}═CR^{V1}CONR^{B1}R^{C1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and each of R^{B1} and R^{C1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B1} taken together with R^{C1} and N forms a substituted or unsubstituted 5- or 6-membered ring.

Preferably, R¹ is —CR^{U1}═CR^{V1}CONR^{B1}R^{C1}, R^{B1} is H and R^{C1} is

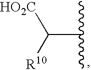

where R¹⁰ is an substituted or unsubstituted C₁₋₆ alkyl, substituted or unsubstituted C₆ or C₁₀ aryl, substituted or unsubstituted C₇₋₁₆ aralkyl, substituted or unsubstituted C₁₋₉ heterocyclyl, or C₂₋₁₅ heterocyclylalkyl.

In another example, R¹ is —CR^{U1}═CR^{V1}CONR^{B1}R^{C1}, W is CH, X¹ is NR⁶, each of R² and R³ is H, and R⁴ is —CH₂CO₂R^{A4}, —CH₂CONR^{B4}R^{C4}, —CH₂S(O)₂R^{D4}, —CH₂S(O)₂NR^{E4}R^{F4}, —CH₂C(O)R^{K4}, or —CH₂C(O)R^{N4}, where each of R^{A4}, R^{B4}, R^{C4}, R^{D4}, R^{E4}, R^{F4}, R^{K4}, and R^{N4} is as previously defined and R^{K4} is a peptide chain of 1-4 natural or unnatural alpha-amino acid residues, where the peptide is linked via its N-terminal amine to the CO group.

In yet another example, R¹ is —CR^{U1}═CR^{V1}CONR^{B1}R^{C1}, W is CH, X¹ is NR⁶, each of R² and R³ is H, and R⁴ is —(CR^{Y4}R^{Z4})CO₂R^{A4}, —(CR^{Y4}R^{Z4})CONR^{B4}R^{C4}, —(CR^{Y4}R^{Z4})S(O)₂R^{D4}, —(CR^{Y4}R^{Z4})S(O)₂NR^{E4}R^{F4}, —(CR^{Y4}R^{Z4})C(O)R^{K4}, —(CR^{Y4}R^{Z4})C(O)R^{N4}, —(CR^{Y4}R^{Z4})NR^{G4}R^{H4}, or —(CR^{Y4}R^{Z4})NR^{b4}C(O)NR^{E4}R^{F4}, where each of R^{A4}, R^{B4}, R^{b4}, R^{C4}, R^{D4}, R^{E4}, R^{F4}, R^{G4}, R^{H4}, R^{N4}, R^{Y4}, and R^{Z4} is as previously defined.

In another embodiment, R¹ is —(CH₂)_{q}COR^{K1}, where q is an integer of from zero to four and R^{K1} is a peptide chain of 1-4 natural or unnatural alpha-amino acid residues, where the peptide is linked via its N-terminal amine to the CO group.

In another embodiment, X¹ is NR⁶, where R⁶ is as previously defined. In a desirable example X¹ is NH and R³ is H.

In another embodiment, R⁴ is —CH₂CO₂R^{A4}, —CH₂CONR^{B4}R^{C4}, —CH₂S(O)₂R^{D4}, —CH₂S(O)₂NR^{E4}R^{F4}, —CH₂C(O)R^{N4}, or —CH₂C(O)R^{K4}, where each of R^{A4}, R^{B4}, R^{C4}, R^{D4}, R^{E4}, R^{F4}, and R^{N4} is as previously defined, and R^{K4} is a peptide chain of 1-4 natural or unnatural alpha-amino acid residues, where the peptide is linked via its N-terminal amine to the CO group.

In another embodiment, R⁴ is —CR^{U4}═CHCONR^{B4}R^{C4}, where R^{U4} is (a) hydrogen, (b) substituted or unsubstituted C₁₋₆ alkyl, (c) substituted or unsubstituted C₆ or C₁₀ aryl, (d) substituted or unsubstituted C₇₋₁₆ aralkyl, (e) substituted or unsubstituted C₁₋₉ heterocyclyl, or (f) substituted or unsubstituted C₂₋₁₅ heterocyclylalkyl, R^{B4} is H or substituted or unsubstituted C₁₋₆ alkyl, and R^{C4} is a substituted or unsubstituted C₁₋₆ alkyl, substituted or unsubstituted C₃₋₈ cycloalkyl, substituted or unsubstituted C₂₋₆ alkenyl, substituted or unsubstituted C₆ or C₁₀ aryl, substituted or unsubstituted C₇₋₁₆ arylalkyl, substituted or unsubstituted C₁₋₉ heterocyclyl, or substituted or unsubstituted C₂₋₁₅ heterocyclylalkyl, or R^{B4} taken together with R^{C4} and N forms an optionally substituted 5- or 6-membered ring.

In yet another embodiment, R⁴ is a substituted or unsubstituted C₆ or C₁₀ aryl, substituted or unsubstituted C₇₋₁₆ arylalkyl, substituted or unsubstituted C₁₋₉heterocyclyl or substituted or unsubstituted C₂₋₁₅heterocyclylalkyl. Preferably, R⁴ is selected from the group consisting of:

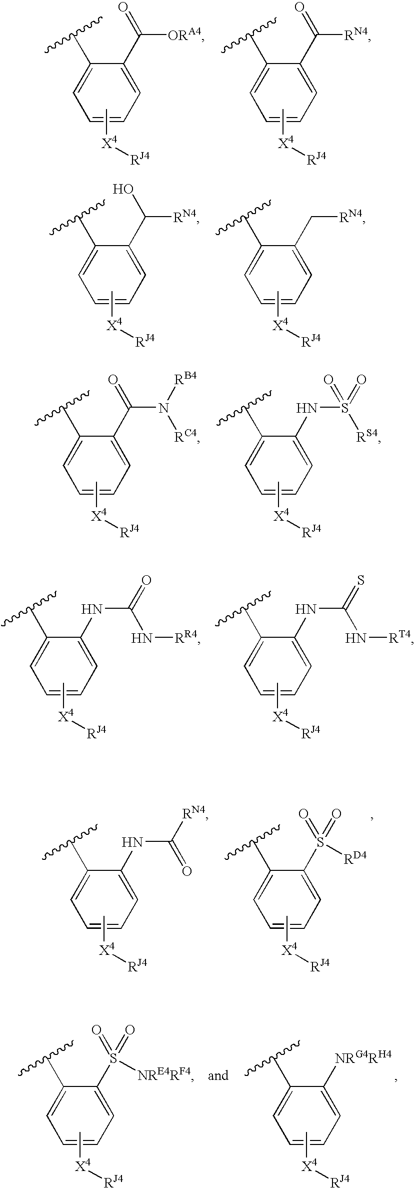

where each of R^{A4}, R^{B4}, R^{C4}, R^{F4}, R^{G4}, R^{H4}, R^{N4}, R^{R4}, R^{R4}, R^{S4} and R^{T4} is, independently hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms; X⁴ is O, S, NR¹⁴, or does not exist, wherein R¹⁴ is hydrogen or a substituted or unsubstituted C₁₋₆ alkyl; and R^{J4} is hydrogen, NO₂, SO₃H, CO₂H, substituted or unsubstituted C₁₋₆ alkyl, substituted or unsubstituted C₁₋₆ alkenyl, substituted or unsubstituted C₇₋₁₆ aralkyl, substituted or unsubstituted C₈₋₁₆ aralkenyl, substituted or unsubstituted C₂₋₁₅ heteroaralkyl, substituted or unsubstituted C₃₋₁₅ heteroaralkenyl, substituted or unsubstituted C₂₋₇ acyl, substituted or unsubstituted C₇₋₁₁ aroyl, substituted or unsubstituted C₃₋₁₀ heteroaroyl, substituted or unsubstituted C₂₋₇ alkoxycarbonyl, substituted or unsubstituted C₄₋₉ cycloalkoxycarbonyl, substituted or unsubstituted C₈₋₁₇ aralkoxycarbonyl, substituted or unsubstituted C₇ or C₁₁ aryloxycarbonyl, substituted or unsubstituted C₃₋₁₆ heteroaralkyloxycarbonyl, substituted or unsubstituted C₂₋₁₀ heterocyclyloxycarbonyl, aminocarbonyl, substituted or unsubstituted C₂₋₇ alkylaminocarbonyl, substituted or unsubstituted C₃₋₁₃ dialkylaminocarbonyl, substituted or unsubstituted C₄₋₉ cycloalkylaminocarbonyl, substituted or unsubstituted C₈₋₁₇ aralkylaminocarbonyl, substituted or unsubstituted C₇ or C₁₁ arylaminocarbonyl, substituted or unsubstituted C₃₋₁₀ heterocyclylaminocarbonyl, C₃₋₁₆ heteroaralkylaminocarbonyl, substituted or unsubstituted C₂₋₇ alkylthiocarbonyl, substituted or unsubstituted C₄₋₉ cycloalkylthiocarbonyl, substituted or unsubstituted C₇₋₁₁ arylthiocarbonyl, substituted or unsubstituted C₈₋₁₇ aralkylthiocarbonyl, substituted or unsubstituted C₂₋₁₀ heterocyclylthiocarbonyl, substituted or unsubstituted C₃₋₁₆ heteroaralkylthiocarbonyl, substituted or unsubstituted C₁₋₆ alkylsulfonyl, substituted or unsubstituted C₃₋₈ cycloalkylsulfonyl, substituted or unsubstituted C₇₋₁₆ aralkylsulfonyl, substituted or unsubstituted C₆ or C₁₀ arylsulfonyl, substituted or unsubstituted C₂₋₉ heterocyclylsulfonyl, or a substituted or unsubstituted C₂₋₁₅ heteroaralkylsulfonyl, with the proviso that R^{J4} is not SO₃H or CO₂H when X⁴ is O or S.

In another aspect, the invention features a compound of formula (II):

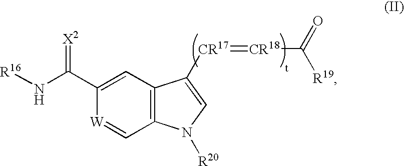

or a derivative, isomer, pharmaceutically acceptable salt, pharmaceutically acceptable solvate, pharmaceutically active metabolite, or prodrug thereof, where

t is 0 or 1;

W is N or CR²¹, where R²¹ is H, halo, amino, hydroxy, substituted or unsubstituted C₁₋₆ alkoxy, substituted or unsubstituted C₁₋₆ alkyl, or forms a 5- or 6-membered ring with R²² via a C₁ or C₂ linkage;

X² is (H,H) or NR²², where R²² is H, C₁₋₆ alkyl, OH, NH₂, NO₂, CO₂R^{22a}, where R^{22a} is C₁₋₆ alkyl, or R²² taken together with R¹⁶ or R²¹ forms a 5- or 6-membered ring via a C₁ or C₂ linkage;

R¹⁶ is H, substituted or unsubstituted C₁₋₆ alkyl, or when taken together with R²² forms a 5- or 6-membered ring via a C₁ or C₂ linkage;

each of R¹⁷ and R¹⁸ is, independently H, halo, or C₁₋₆ alkyl;

R¹⁹ is C₁₋₆ alkyl C₃₋₈ cycloalkyl OR²³, or NR²³R²⁴, where each of R²³ or R²⁴ is, independently, H, substituted or unsubstituted C₁₋₆ alkyl, C₃₋₈ cycloalkyl, or C₂₋₆ alkenyl, or R²³ and R²⁴ taken together with N forms a substituted or unsubstituted 5- or 6-membered ring;

R²⁰ is a substituted or unsubstituted C₂₋₇ alkanoyl, substituted or unsubstituted C₁₋₆ alkyl, substituted or unsubstituted C₂₋₆ alkenyl, substituted or unsubstituted C₁₋₆ alkoxy, substituted or unsubstituted C₂₋₁₂ alkoxyalkyl, where the alkylene group is of one to six carbon atoms, substituted or unsubstituted C₁₋₆ alkylsulfinyl, substituted or unsubstituted C₂₋₁₂ alkylsulfinylalkyl, where the alkylene group is of one to six carbon atoms, substituted or unsubstituted C₁₋₆ alkylsulfonyl, substituted or unsubstituted C₆ or C₁₀ aryl, substituted or unsubstituted C₇₋₁₆ arylalkyl, where the alkylene group is of one to six carbon atoms, C₁₋₆ aminoalkyl, substituted or unsubstituted C₇ or C₁₁ aryloyl, substituted or unsubstituted C₃₋₈ cycloalkyl, substituted or unsubstituted C₄₋₁₄ cycloalkylalkyl, where the alkylene group is of one to six carbon atoms, substituted or unsubstituted C₁₋₉ heterocyclyl, C₂₋₁₅ heterocyclylalkyl, substituted or unsubstituted C₁₋₉ (heterocyclyl)oxy, substituted or unsubstituted C₂₋₁₀ (heterocyclyl)oyl, substituted or unsubstituted C₁₋₆ thioalkoxy, substituted or unsubstituted C₂₋₁₂ thioalkoxyalkyl, where the alkylene group is of one to six carbon atoms, substituted or unsubstituted C₆ or C₁₀ aryloxy, substituted or unsubstituted C₃₋₈ cycloalkoxy, substituted or unsubstituted C₄₋₁₄ cycloalkylalkoxy, substituted or unsubstituted C₇₋₁₆ arylalkoxy, amidino, guanidino, ureido, —(CH₂)_{q}CO₂R^{A20}, wherein q is an integer of from zero to four and R^{A20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}COR^{K20}, wherein q is an integer of from zero to four and R^{K20} is a peptide chain of 1-4 natural or unnatural alpha-amino acid residues, where the peptide is linked via its N-terminal amine to the CO group, —(CH₂)_{q}COR^{N20}, wherein q is an integer of from zero to four and R^{N20} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CONR^{B20}R^{C20}, wherein q is an integer of from zero to four and each of R^{B20} and R^{C20} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B20} taken together with R^{C20} and N forms a substituted or unsubstituted 5- or 6-membered ring, —(CH₂)_{q}S(O)₂R^{D20}, wherein q is an integer of from zero to four and R^{D20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide of 1-4 natural or unnatural alpha-amino acid residues, where the peptide is linked via its N-terminal amine to the S(O)₂ group, —(CH₂)_{q}S(O)₂NR^{E20}R^{F20}, wherein q is an integer of from zero to four and each of R^{E20} and R^{F20} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E20} taken together with R^{F20} and N forms a substituted or unsubstituted 5- or 6-membered ring, —(CH₂)_{q}CR^{U20}═CR^{V20}CO₂R^{A20}, wherein q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and R^{A20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CR^{U2}O═CR^{V20}COR^{K20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and R^{K20} is a peptide chain of 1-4 natural or unnatural alpha-amino acid residues, where the peptide is linked via its N-terminal amine to the CO group, —(CH₂)_{q}CR^{U20}═CR^{V20}COR^{N20}, where q is an integer of from zero to four, each of R^{U20} and R^{V20} is, independently, (a) hydrogen, (b) halogen, (c) hydroxyl, (d) substituted or unsubstituted C₁₋₆alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and R^{N20} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CR^{U20}═CR^{V20}CONR^{B20}R^{C20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and each of R^{B20} and R^{C20} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B20} taken together with R^{C20} and N forms a substituted or unsubstituted 5- or 6-membered ring, —(CH₂)_{q}CR^{U20}═CR^{V20}S(O)₂R^{D20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂—X₅ heterocyclylalkyl, and R^{D20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, —(CH₂)_{q}CR^{U20}═CR^{V20}S(O)₂NR^{E20}R^{F20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and each of R^{E20} and R^{F20} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E20} taken together with R^{F20} and N forms a substituted or unsubstituted 5- or 6-membered ring, —(CH₂)_{q}NR^{G20}R^{H20}, where q is an integer of from zero to four and each of R^{G20} and R^{H20} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N20}, where R^{N20} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A20}, where R^{A20} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) (CH₂)_{q}OR^{A20}, where q is an integer of from 0 to 4 and R^{A20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, (o) —S(O)₂R^{S20}, where R^{S20} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (p) a peptide chain of 1-4 natural or unnatural alpha-amino acid residues, where the peptide is linked via its C-terminal carboxy group to N, with the proviso that no two groups are bound to the nitrogen atom through a carbonyl group or a sulfonyl group, or —NR^{b20}CONR^{E20}R^{F20}, where R^{b20} is hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and each of R^{E20} and R^{F20} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E20} taken together with R^{F20} and N forms a substituted or unsubstituted 5- or 6-membered ring.

In another aspect, the invention features a pharmaceutical composition that includes a pharmaceutically acceptable carrier and a therapeutically effective amount of any compound of the invention, or a pharmaceutically acceptable salt or prodrug thereof.

The invention also features a method of treating a patient in need of thromboembolic disorder treatment that includes administering to the patient a therapeutically effective amount of any compound of the invention, or a pharmaceutically acceptable salt or prodrug thereof. The thromboembolic disorder can be arterial cardiovascular thromboembolic disorders, venous cardiovascular thromboembolic disorders, and thromboembolic disorders in the chambers of the heart; including unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In addition to their use in anticoagulant therapy, Factor XIa inhibitors are useful in the treatment and prevention of other diseases in which the generation of thrombin has been implicated as playing a physiologic role. For example, thrombin has been implicated in contributing to the morbidity and mortality of chronic and degenerative diseases such as cancer, arthritis, atherosclerosis, and Alzheimer's disease by its ability to regulate many different cell types through specific cleavage and activation of a cell surface thrombin receptor, mitogenic effects, diverse cellular functions such as cell proliferation, for example, abnormal proliferation of vascular cells resulting in restenosis or angiogenesis, release of PDGF, and DNA synthesis. Inhibition of Factor XIa effectively blocks thrombin generation and therefore neutralizes any physiologic effects of thrombin on various cell types. The representative indications discussed above include some, but not all, of the potential clinical situations amenable to treatment with a Factor XIa inhibitor.

The terms “acyl” or “alkanoyl,” as used interchangeably herein, represent an alkyl group, as defined herein, or hydrogen attached to the parent molecular group through a carbonyl group, as defined herein, and is exemplified by formyl, acetyl, propionyl, butanoyl and the like. Exemplary unsubstituted acyl groups are of from 2 to 7 carbons.

The term “acylamino,” as used herein, represents an acyl group attached to the parent molecular group through a nitrogen atom. Exemplary unsubstituted acylamino groups are of from 2 to 7 carbons.

The term “acyloxy,” as used herein, represents an acyl group attached to the parent molecular group through an oxygen atom. Exemplary unsubstituted acyloxy groups have from 2 to 7 carbons.

The term “alkenyl,” as used herein, represents monovalent straight or branched chain groups of, unless otherwise specified, from 2 to 6 carbons containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) amino; (5) aryl; (6) arylalkoxy, where the alkylene group is of one to six carbon atoms; (7) aryloyl; (8) azido; (9) carboxaldehyde; (10) cycloalkyl of three to eight carbon atoms; (11) halo; (12) heterocycle; (13) (heterocycle)oxy; (14) (heterocycle)oyl; (15) hydroxy; (16) N-protected amino; (17) nitro; (18) oxo; (19) spiroalkyl of three to eight carbon atoms; (20) thioalkoxy of one to six carbon atoms; (21) thiol; (22) —CO₂R^{A}, where R^{A} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (23) —C(O)NR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (24) —S(O)₂R^{D}, where R^{D} is selected from the group consisting of (a) alkyl, (b) aryl and (c) arylalkyl, where the alkylene group is of one to six carbon atoms; (25) —S(O)₂NR^{E}R^{F}, where each of R^{E} and RF is, independently, selected from the group consisting of (a) hydrogen, (b) alkyl, (c) aryl and (d) arylalkyl, where the alkylene group is of one to six carbon atoms; and (26) —NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group.

The terms “alkoxy” or “alkyloxy,” as used interchangeably herein, represent an alkyl group attached to the parent molecular group through an oxygen atom. Exemplary unsubstituted alkoxy groups are of from 1 to 6 carbons.

The terms “alkoxyalkyl” or “alkyloxyalkyl,” as used interchangeably herein, represent an alkyl group to which is attached an alkoxy group. Exemplary unsubstituted alkoxyalkyl groups are of from 2 to 12 carbons.

The terms “alkoxycarbonyl” or “alkyloxycarbonyl,” as used interchangeably herein, represent an ester group; i.e. an alkoxy group, attached to the parent molecular group through a carbonyl group and is exemplified by methoxycarbonyl, ethoxycarbonyl and the like. Exemplary unsubstituted alkoxycarbonyl groups are of from 2 to 7 carbons.

The term “alkyl,” as used herein, represents a monovalent group derived from a straight or branched chain saturated hydrocarbon of, unless otherwise specified, from 1 to 6 carbons and is exemplified by methyl, ethyl, n- and iso-propyl, n-, sec-, iso- and tert-butyl, neopentyl and the like and may be optionally substituted with one, two, three or, in the case of alkyl groups of two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) amino; (5) aryl; (6) arylalkoxy; (7) aryloyl; (8) azido; (9) carboxaldehyde; (10) cycloalkyl of three to eight carbon atoms; (11) halo; (12) heterocyclyl; (13) (heterocycle)oxy; (14) (heterocycle)oyl; (15) hydroxyl; (16) N-protected amino; (17) nitro; (18) oxo; (19) spiroalkyl of three to eight carbon atoms; (20) thioalkoxy of one to six carbon atoms; (21) thiol; (22) —CO₂R^{A}, where R^{A} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, (23) —C(O)NR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (24) —S(O)₂R^{D}, where R^{D} is selected from the group consisting of (a) alkyl, (b) aryl and (c) arylalkyl, where the alkylene group is of one to six carbon atoms; (25) —S(O)₂NR^{E}R^{F}, where each of R^{E} and RF is, independently, selected from the group consisting of (a) hydrogen, (b) alkyl, (c) aryl and (d) arylalkyl, where the alkylene group is of one to six carbon atoms; and (26) —NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group.

The term “alkylamino,” as used herein, represents an alkyl group attached to the parent molecular group through a nitrogen atom. Exemplary unsubstituted alkylamino groups are of from 1 to 6 carbons.

The term “alkylaminocarbonyl,” as used herein, represents an alkylamino group attached to the parent molecular group through a carbonyl group. Exemplary unsubstituted alkylaminocarbonyl groups are of from 2 to 7 carbons.

The term “alkylaminosulfonyl,” as used herein, represents an alkylamino group attached to the parent molecular group through an —S(O)₂— group. Exemplary unsubstituted alkylaminosulfonyl groups are of from 1 to 6 carbons.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkylsulfinyl,” as used herein, represents an alkyl group attached to the parent molecular group through an —S(O)— group. Exemplary unsubstituted alkylsulfinyl groups are of from 1 to 6 carbons.

The term “alkylsulfinylalkyl,” as used herein, represents an alkyl group, as defined herein, substituted by an alkylsulfinyl group. Exemplary unsubstituted alkylsulfinylalkyl groups are of from 2 to 12 carbons.

The term “alkylsulfonyl,” as used herein, represents an alkyl group attached to the parent molecular group through an —S(O)₂— group. Exemplary unsubstituted alkylsulfonyl groups are of from 1 to 6 carbons.

The term “alkylsulfonylalkyl,” as used herein, represents an alkyl group, as defined herein, substituted by a alkylsulfonyl group. Exemplary unsubstituted alkylsulfonylalkyl groups are of from 2 to 12 carbons.

The term “alkylthio,” as used herein, represents an alkyl group attached to the parent molecular group through a sulfur atom. Exemplary unsubstituted alkylthio groups are of from 1 to 6 carbons.

The term “alkynyl,” as used herein, represents monovalent straight or branched chain groups of from two to six carbon atoms containing a carbon-carbon triple bond and is exemplified by ethynyl, 1-propynyl, and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) amino; (5) aryl; (6) arylalkoxy, where the alkylene group is of one to six carbon atoms; (7) aryloyl; (8) azido; (9) carboxaldehyde; (10) cycloalkyl of three to eight carbon atoms; (11) halo; (12) heterocycle; (13) (heterocycle)oxy; (14) (heterocycle)oyl; (15) hydroxy; (16) N-protected amino; (17) nitro; (18) oxo; (19) spiroalkyl of three to eight carbon atoms; (20) thioalkoxy of one to six carbon atoms; (21) thiol; (22) —CO₂R^{A}, where R^{A} is selected from the group consisting of (a) alkyl, (b) aryl and (c) arylalkyl, where the alkylene group is of one to six carbon atoms; (23) —C(O)NR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (24) —S(O)₂R^{D}, where R^{D} is selected from the group consisting of (a) alkyl, (b) aryl and (c) arylalkyl, where the alkylene group is of one to six carbon atoms; (25) —S(O)₂NR^{E}R^{F}, where each of R^{E} and RF is, independently, selected from the group consisting of (a) hydrogen, (b) alkyl, (c) aryl and (d) arylalkyl, where the alkylene group is of one to six carbon atoms; and (26) —NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group.

The term “alpha-amino acid residue,” as used herein, represents a —N(R^{A})C(R^{B})(R^{C})C(O)— linkage, where R^{A} is selected from the group consisting of (a) hydrogen, (b) alkyl, (c) aryl and (d) arylalkyl, as defined herein; and each of R^{B} and R^{C} is, independently, selected from the group consisting of: (a) hydrogen, (b) optionally substituted alkyl, (c) optionally substituted cycloalkyl, (d) optionally substituted aryl, (e) optionally substituted arylalkyl, (f) optionally substituted heterocyclyl, and (g) optionally substituted heterocyclylalkyl, each of which is as defined herein. For natural amino acids, R^{B} is H and R^{C} corresponds to those side chains of natural amino acids found in nature, or their antipodal configurations. Exemplary natural amino acids include alanine, cysteine, aspartic acid, glutamic acid, phenylalanine, glycine, histidine, isoleucine, lysine, leucine, methionine, aspartamine, ornithine, proline, glutamine, arginine, serine, threonine, valine, tryptophan, and tyrosine, each of which, except glycine, as their D- or L-form. As used herein, for the most part, the names of naturally-occurring amino acids and aminoacyl residues used herein follow the naming conventions suggested by the IUPAC Commission on the Nomenclature of Organic Chemistry and the IUPAC-IUB Commission on Biochemical Nomenclature as set out in Nomenclature of α-Amino Acids (Recommendations, 1974), Biochemistry 14 (2), 1975. The present invention also contemplates non-naturally occurring (i.e., unnatural) amino acid residues in their D- or L-form such as, for example, homophenylalanine, phenylglycine, cyclohexylglycine, cyclohexylalanine, cyclopentyl alanine, cyclobutylalanine, cyclopropylalanine, cyclohexylglycine, norvaline, norleucine, thiazoylalanine (2-, 4- and 5-substituted), pyridylalanine (2-, 3- and 4-isomers), naphthalalanine (1- and 2-isomers) and the like. Stereochemistry is as designated by convention, where a bold bond indicates that the substituent is oriented toward the viewer (away from the page) and a dashed bond indicates that the substituent is oriented away from the viewer (into the page). If no stereochemical designation is made, it is to be assumed that the structure definition includes both stereochemical possibilities.

The term “amidine,” as used herein, represents an —C(═NH)NH₂ group.

The term “amino,” as used herein, represents an —NH₂ group.

The term “aminoalkyl,” as used herein, represents an alkyl group, as defined herein, substituted by an amino group.

The term “aryl,” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl and the like and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) cycloalkylalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) —(CH₂)_{q}CONR^{B}R^{C}, where R^{B} and R^{C} are independently selected from the group consisting of (a) hydrogen, (b) alkyl, (c) aryl and (d) arylalkyl, where the alkylene group is of one to six carbon atoms; (38) —(CH₂)_{q}S(O)₂R^{D}, where R^{D} is selected from the group consisting of (a) alkyl, (b) aryl and (c) arylalkyl, where the alkylene group is of one to six carbon atoms; (39) —(CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The terms “arylalkenyl” or “aralkenyl,” as used interchangeably herein, represent an aryl group attached to the parent molecular group through an alkenyl group. Exemplary unsubstituted arylalkenyl groups are of from 8 to 16 carbons.

The terms “arylalkoxy” or “aralkoxy,” as used interchangeably herein, represent an arylalkyl group attached to the parent molecular group through an oxygen atom. Exemplary unsubstituted arylalkoxy groups are of from 7 to 16 carbons.

The terms “arylalkoxycarbonyl” or “aralkoxycarbonyl,” as used interchangeably herein, represent an arylalkoxy group attached to the parent molecular group through a carbonyl group. Exemplary unsubstituted arylalkoxycarbonyl groups are of from 8 to 17 carbons.

The terms “arylalkyl” or “aralkyl,” as used interchangeably herein, represent an aryl group attached to the parent molecular group through an alkyl group. Exemplary unsubstituted arylalkyl groups are of from 7 to 16 carbons.

The terms “arylalkylamino” or “aralkylamino,” as used interchangeably herein, represent an arylalkyl group attached to the parent molecular group through a nitrogen atom. Exemplary unsubstituted arylalkylamino groups are of from 7 to 16 carbons.

The terms “arylalkylaminocarbonyl” or “aralkylaminocarbonyl,” as used interchangeably herein, represents an arylalkylamino group attached to the parent molecular group through a carbonyl group. Exemplary unsubstituted arylalkylaminocarbonyl groups are of from 8 to 17 carbons.

The terms “arylalkylsulfinyl” or “aralkylsulfinyl,” as used interchangeably herein, represent an arylalkyl group attached to the parent molecular group through an —SO— group. Exemplary unsubstituted arylalkylsulfinyl groups are of from 7 to 16 carbons.

The terms “arylalkylsulfonyl” or “aralkylsulfonyl,” as used interchangeably herein, represent an aralkyl group attached to the parent molecular group through an —S(O)₂— group. Exemplary unsubstituted arylalkylsulfonyl groups are of from 7 to 16 carbons.

The term “arylalkylthio” or “aralkylthio,” as used interchangeably herein, represents an arylalkyl group attached to the parent molecular group through a sulfur atom. Exemplary unsubstituted arylalkylthio groups are of from 7 to 16 carbons.

The term “arylamino,” as used herein, represents an aryl group which is attached to the parent molecular group through a nitrogen atom. Exemplary unsubstituted arylamino groups are of 6 or 10 carbons.

The term “arylaminocarbonyl,” as used herein, represents an arylamino group attached to the parent molecular group through a carbonyl group. Exemplary unsubstituted arylaminocarbonyl groups are of from 7 or 11 carbons.

The term “arylaminosulfonyl,” as used herein, represents an arylamino group attached to the parent molecular group through an —S(O)₂— group. Exemplary unsubstituted arylaminosulfonyl groups are of 6 or 10 carbons.

The term “aryloxy,” as used herein, represents an aryl group which is attached to the parent molecular group through an oxygen atom. Exemplary unsubstituted aryloxy groups are of 6 or 10 carbons.

The term “aryloxycarbonyl,” as used herein, represents an aryloxy group which is attached to the parent molecular group through a carbonyl group. Exemplary unsubstituted aryloxycarbonyl groups are of 7 or 11 carbons.

The terms “aryloyl” or “aroyl,” as used interchangeably herein, represent an aryl group which is attached to the parent molecular group through a carbonyl group. Exemplary unsubstituted aryloxycarbonyl groups are of 7 or 11 carbons.

The terms “aryloylamino” or “aroylamino,” as used interchangeably herein, represent an aroyl group which is attached to the parent molecular group through a nitrogen atom. Exemplary unsubstituted aryloylamino groups are of 7 or 11 carbons.

The term “arylsulfinyl,” as used herein, represents an aryl group attached to the parent molecular group through an —SO— group. Exemplary unsubstituted arylsulfinyl groups are of 6 or 10 carbons.

The term “arylsulfonyl,” as used herein, represents an aryl group attached to the parent molecular group through an —S(O)₂— group. Exemplary unsubstituted arylsulfonyl groups are of 6 or 10 carbons.

The term “arylthio,” as used herein, represents an aryl group which is attached to the parent molecular group through a sulfur atom. Exemplary unsubstituted arylthio groups are of 6 or 10 carbons.

The term “azido,” as used herein, represents an —N₃ group.

The term “azidoalkyl,” as used herein, represents an alkyl group, as defined herein, substituted by an azido group.

By “blood component” is meant a biological entity normally found in blood, such as, for example cells, such as erythrocytes, leukocytes, and platelets, or proteins such as immunoglobulins, serum albumin, ferritin, steroid binding proteins, such as corticosteroid-binding globulin and sex hormone-binding globulin, transferrin, thyroxin binding protein, and alpha-2-macroglobulin. Blood components also include glycans, including glycosylamino glycans. Preferred blood components are those that have reactive organic functionality, such as thiols or amines.

The terms “carbamate” or “carbamyl,” as used interchangeably herein, represent a R^{A}OC(O)NR^{B}— group, or a —OC(O)NR^{B}— linkage, depending on the chemical context in which this term is used, where R^{A} is selected from the group consisting of (a) alkyl, (b) aryl, and (c) arylalkyl; and R^{B} is selected from the group consisting of (a) hydrogen, (b) alkyl, (c) aryl and (d) arylalkyl, as defined herein.

The term “carbonate,” as used herein represents a —R^{A}OC(O)O— group, or a —OC(O)O— linkage, depending on the chemical context in which this term is used, where R^{A} is selected from the group consisting of (a) alkyl, (b) aryl, and (c) arylalkyl, as defined herein.

The term “carbonyl,” as used herein, represents a C═O group.

The term “carboxaldehyde,” as used herein, represents a —CHO group.

The term “(carboxaldehyde)alkyl,” as used herein, represents an alkyl group, as defined herein, substituted by a carboxaldehyde group.

The term “carboxy,” as used herein, represents a —CO₂H group.

The term “carboxyalkyl,” as used herein, represents an alkyl group, as defined herein, substituted by a carboxy group.

The term “cycloalkenyl,” as used herein represents a monovalent cyclic hydrocarbon of from three to eight carbons, unless otherwise specified, having at least one carbon-carbon double bond. The cycloalkenyl groups of this invention can be optionally substituted with (1) alkanoyl of one to six carbon atoms; (2) alkyl of one to six carbon atoms; (3) alkoxy of one to six carbon atoms; (4) alkoxyalkyl, where die alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) cycloalkylalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (38) —(CH₂)_{q}S(O)₂R^{D}, where R^{D} is selected from the group consisting of (a) alkyl, (b) aryl and (c) arylalkyl, where the alkylene group is of one to six carbon atoms; (39) —(CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “cycloalkyl,” as used herein represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl groups of this invention can be optionally substituted with (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) cycloalkylalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (38) —(CH₂)_{q}S(O)₂R^{D}, where R^{D} is selected from the group consisting of (a) alkyl, (b) aryl and (c) arylalkyl, where the alkylene group is of one to six carbon atoms; (39) —(CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “cycloalkylamino,” as used herein, represents a cycloalkyl group attached to the parent molecular group through a nitrogen atom. Exemplary unsubstituted cycloalkylamino groups are of from 3 to 8 carbons.

The term “cycloalkylaminocarbonyl,” as used herein, represents a cycloalkylamino group attached to the parent molecular group through a carbonyl group. Exemplary unsubstituted cycloalkylaminocarbonyl groups are of from 4 to 9 carbons.

The terms “cycloalkyloxy” or “cycloalkoxy,” as used interchangeably herein, represent a cycloalkyl group, as defined herein, attached to the parent molecular group through an oxygen atom. Exemplary unsubstituted cycloalkyloxy groups are of from 3 to 8 carbons.

The terms “cycloalkyloxycarbonyl” or “cycloalkoxycarbonyl,” as used interchangeably herein, represent a cycloalkyloxy group, as defined herein, attached to the parent molecular group through a carbonyl group. Exemplary unsubstituted cycloalkyloxycarbonyl groups are of from 4 to 9 carbons.

The term “cycloalkylalkoxy,” as used herein, represents an alkoxy group, as defined herein, to which is attached a cycloalkyl group. Exemplary unsubstituted cycloalkylalkoxy groups are of from 4 to 14 carbons.

The term “cycloalkylalkyl,” as used herein, represents a cycloalkyl group, as defined herein, attached to the parent molecular group through an alkyl group. Exemplary unsubstituted cycloalkylalkyl groups are of from 4 to 14 carbons.

The term “cycloalkylsulfinyl,” as used herein, represents a cycloalkyl group attached to the parent molecular group through an —SO— group.

Exemplary unsubstituted cycloalkylsulfinyl groups are of from 3 to 8 carbons.

The term “cycloalkylsulfonyl,” as used herein, represents a cycloalkyl group attached to the parent molecular group through an —S(O)₂— group. Exemplary unsubstituted cycloalkylsulfonyl groups are of from 3 to 8 carbons.

The term “dialkylamino,” as used herein, represents an N,N-dialkylsubstituted amine attached to the parent molecular group through the nitrogen atom. The two alkyl substituents of a dialkylamino group can be the same or different, or can be joined together to form a ring. Exemplary dialkylamino groups are of from 2 to 12 carbons and include dimethylamino, diethylamino, pyrrolidino, and piperidino.

The term “haloalkyl,” as used herein, represents an alkyl group, as defined herein, substituted by one, two, or three halogen atoms and is exemplified by chloromethyl, bromoethyl, trifluoromethyl and the like.

The term “halogen,” as used herein, represents F, Cl, Br and I.

The term “heteroaryl,” as used herein, represents that subset of heterocycles, as defined herein, which are aromatic: i.e., they contain 4n+2 pi electrons within the mono- or multicyclic ring system. Exemplary unsubstituted heteroaryl groups are of from 1 to 9 carbons.

The terms “heteroarylalkenyl” or “heteroaralkenyl,” or as used interchangeably herein, represent a heteroaryl group, as defined herein, attached to the parent molecular group through an alkenyl group. Exemplary unsubstituted heteroarylalkenyl groups are of from 3 to 15 carbons.

The terms “heteroarylalkyl” or “heteroaralkyl,” as used interchangeably herein, represent a heteroaryl group, as defined herein, attached to the parent molecular group through an alkyl group. Exemplary unsubstituted heteroarylalkyl groups are of from 2 to 15 carbons.

The terms “heteroarylalkylamino” or “heteroaralkylamino,” as used interchangeably herein, represent a heteroarylalkyl group, as defined herein, attached to the parent molecular group through a nitrogen atom. Exemplary unsubstituted heteroarylalkylamino groups are of from 2 to 15 carbons.

The terms “heteroarylalkylaminocarbonyl” or “heteroaralkylaminocarbonyl,” or as used interchangeably herein, represent a heteroarylalkylamino group, as defined herein, attached to the parent molecular group through a carbonyl group. Exemplary unsubstituted heteroarylalkylaminocarbonyl groups are of from 3 to 16 carbons.

The terms “heteroaryloyl” or “heteroaroyl,” or as used interchangeably herein, represent a heteroaryl group, as defined herein, attached to the parent molecular group through a carbonyl group. Exemplary unsubstituted heteroaryloyl groups are of from 2 to 10 carbons.

The terms “heteroarylalkyloxy” or “heteroaralkoxy,” or as used interchangeably herein, represent a heteroarylalkyl group, as defined herein, attached to the parent molecular group through an oxygen atom. Exemplary unsubstituted heteroarylalkyloxy groups are of from 2 to 15 carbons.

The terms “heteroarylalkyloxycarbonyl” or “heteroaralkoxycarbonyl,” as used interchangeably herein, represent a heteroaralkoxy group, as defined herein, attached to the parent molecular group through a carbonyl group. Exemplary unsubstituted heteroarylalkyloxycarbonyl groups are of from 3 to 16 carbons.

The terms “heteroarylalkylsulfonyl” or “heteroaralkylsulfonyl,” as used interchangeably herein, represent a heteroarylalkyl group attached to the parent molecular group through an —S(O)₂— group. Exemplary unsubstituted heteroarylalkylsulfonyl groups are of from 2 to 15 carbons.

The term “heteroarylamino,” as used herein, represents a heteroaryl group attached to the parent molecular group through a nitrogen atom. Exemplary unsubstituted heteroarylamino groups are of from 1 to 9 carbons.

The term “heteroarylaminocarbonyl,” as used herein, represents a heteroarylamino group attached to the parent molecular group through a carbonyl group. Exemplary unsubstituted heteroarylaminocarbonyl groups are of from 2 to 10 carbons.

The term “heteroarylaminosulfonyl,” as used herein, represents a heteroarylamino group attached to the parent molecular group through an —S(O)₂— group. Exemplary unsubstituted heteroarylaminosulfonyl groups are of from 1 to 9 carbons.

The term “heteroaryloxy,” as used herein, represents a heteroaryl group attached to the parent molecular group through an oxygen atom. Exemplary unsubstituted heteroaryloxy groups are of from 1 to 9 carbons.

The term “heteroaryloxycarbonyl,” as used herein, represents a heteroaryloxy group attached to the parent molecular group through a carbonyl group. Exemplary unsubstituted heteroaryloxycarbonyl groups are of from 1 to 9 carbons.

The term “heteroarylsulfonyl,” as used herein, represents a heteroaryl group attached to the parent molecular group through an —S(O)₂— group. Exemplary unsubstituted heteroarylsulfonyl groups are of from 1 to 9 carbons.

The term “heteroarylthio,” as used herein, represents a heteroaryl group attached to the parent molecular group through a sulfur atom. Exemplary unsubstituted heteroaryloxy groups are of from 1 to 9 carbons.

The terms “heterocycle” or “heterocyclyl,” as used interchangeably herein represent a 5-, 6- or 7-membered ring, unless otherwise specified, containing one, two, three, or four heteroatoms independently selected from the group consisting of nitrogen, oxygen and sulfur. The 5-membered ring has zero to two double bonds and the 6- and 7-membered rings have zero to three double bonds. The term “heterocycle” also includes bicyclic, tricyclic and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring such as indolyl, quinolyl, isoquinolyl, tetrahydroquinolyl, benzofuryl, benzothienyl and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrotbienyl, dihydrothienyl, dihydroinidolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

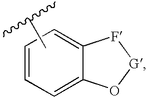

where

F′ is selected from the group consisting of —CH₂—, —CH₂O— and —O—, and G′ is selected from the group consisting of —C(O)— and —(C(R′)(R′″))ᵥ—, where each of R′ and R″ is, independently, selected from the group consisting of hydrogen or alkyl of one to four carbon atoms, and v is one to three and includes groups such as 1,3-benzodioxolyl, 1,4-benzodioxanyl and the like. Any of the heterocycle groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) cycloalkylalkyl, where the cycloalkyl group is of three to eight carbon atoms and the alkylene group is of one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) —(CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) —(CH₂)_{q}CONR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (38) —(CH₂)_{q}S(O)₂R^{D}, where R^{D} is selected from the group consisting of (a) alkyl, (b) aryl and (c) arylalkyl, where the alkylene group is of one to six carbon atoms; (39) —(CH₂)_{q}S(O)₂NR^{E}R^{F}, where each of R^{E} and RF is, independently, selected from the group consisting of (a) hydrogen, (b) alkyl, (c) aryl and (d) arylalkyl, where the alkylene group is of one to six carbon atoms; (40) —(CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) cycloalkylalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “heterocyclylalkyl” represents a heterocyclyl group attached to the parent molecular group through an alkyl group. Exemplary unsubstituted heterocyclylalkyl groups are of from 2 to 15 carbons.

The terms “heterocyclylamino” or “(heterocycle)amino,” as used interchangeably herein, represents a heterocycle group, as defined herein, attached to the parent molecular group through nitrogen. Exemplary unsubstituted heterocyclylamino groups are of from 1 to 9 carbons.

The terms “heterocyclyloxy” or “(heterocycle)oxy,” as used interchangeably herein, represents a heterocycle group, as defined herein, attached to the parent molecular group through an oxygen atom. Exemplary unsubstituted heterocyclyloxy groups are of from 1 to 9 carbons.

The terms “heterocyclyloxycarbonyl” or “(heterocycle)oxycarbonyl,” as used interchangeably herein, represents a heterocycloxy group, as defined herein, attached to the parent molecular group through a carbonyl group. Exemplary unsubstituted heterocyclyloxycarbonyl groups are of from 2 to 10 carbons.

The term “heterocyclyloyl” or “(heterocycle)oyl,” as used interchangeably herein, represents a heterocycle group, as defined herein, attached to the parent molecular group through a carbonyl group. Exemplary unsubstituted heterocyclyloyl groups are of from 2 to 10 carbons.

The term “heterocyclylsulfonyl,” as used herein, represents a heterocyclyl group attached to the parent molecular group through an —S(O)₂— group. Exemplary unsubstituted heterocyclylsulfonyl groups are of from 1 to 9 carbons.

The term “heterocyclylthio,” as used herein, represents a heterocyclyl group attached to the parent molecular group through a sulfur atom. Exemplary unsubstituted heteroaryloxy groups are of from 1 to 9 carbons.

The term “hydroxy” as used herein, represents an —OH group.

The term “hydroxyalkyl,” as used herein, represents an alkyl group, as defined herein, substituted by one to three hydroxy groups, with the proviso that no more than one hydroxy group may be attached to a single carbon atom of the alkyl group and is exemplified by hydroxymethyl, dihydroxypropyl and the like.

The term “methine” as used herein, represents a ═C(H)— group.

The terms “N-protected amidino” or “protected amidino,” as used interchangeably herein, refers to an amidino group, as defined herein, to which is attached an N-protecting or nitrogen-protecting group, as defined herein. Preferable amidine protection includes one or two Boc protecting groups, one or two Cbz protecting groups, a trityl protecting group, or a protection with a trityl analog (such as, for example, chlorotrityl or methoxytrityl). In addition, the amidine protecting group can serve as a handle for the solid-phase support of amidine-containing intermediates in which the intermediate is linked to the resin via a labile moiety, such as for example, a carbamate or a trityl moiety. These intermediates are useful for the preparation of compounds of the invention via solid-phase synthesis routes.

The term “N-protected amino,” as used herein, refers to an amino group, as defined herein, to which is attached an N-protecting or nitrogen-protecting group, as defined herein.

The term “N-protected aminoalkyl,” as used herein, refers to an alkyl group, as defined herein, which is substituted by an N-protecting or nitrogen-protecting group, as defined herein.

The terms “N-protecting group” or “nitrogen protecting group” as used herein, represent those groups intended to protect an amino group against undersirable reactions during synthetic procedures. Commonly used N-protecting groups are disclosed in Greene, “Protective Groups In Organic Synthesis, 3^{rd} Edition” (John Wiley & Sons, New York, 1999), which is incorporated herein by reference. N-protecting groups comprise acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and chiral auxiliaries such as protected or unprotected D, L or D, L-amino acids such as alanine, leucine, phenylalanine and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl and the like, arylalkyl groups such as benzyl, triphenylmethyl, benzyloxymethyl and the like and silyl groups such as trimethylsilyl and the like. Preferred N-protecting groups are formyl, acetyl, benzoyl, pivaloyl, t-butylacetyl, alanyl, phenylsulfonyl, benzyl, t-butyloxycarbonyl (Boc) and benzyloxycarbonyl (Cbz).

The term “nitro,” as used herein, represents an —NO₂ group.

The term “nitroalkyl,” as used herein, represents an alkyl group substituted by an —NO₂ group.

The term “oxo,” as used herein, represents ═O.

The term “perfluoroalkyl,” as used herein, represents an alkyl group, as defined herein, where each hydrogen radical bound to the alkyl group has been replaced by a fluoride radical. Perfluoroalkyl groups are exemplified by trifluoromethyl, pentafluoroethyl, and the like.

The term “perfluoroalkoxy,” as used herein, refers to a perfluoroalkyl group, as defined herein, attached to the parent molecular group through an oxygen atom.

The term “pharmaceutically acceptable salt,” as use herein, represents those salts which are, within the scope of sound medical judgement, suitable for use in contact with the tissues of humans and animals without undue toxicity, irritation, allergic response and the like and are commensurate with a reasonable benefit/risk ratio. Pharmaceutically acceptable salts are well known in the art. For example, S. M Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like. The term “pharmaceutically acceptable ester,” as used herein, represents esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl group preferably has not more than 6 carbon atoms. Examples of particular esters includes formates, acetates, propionates, butyates, acrylates and ethylsuccinates.

The term “pharmaceutically active metabolite” as used herein, means a biologically active sustance resulting from one or more in vivo processing steps on a compound of the invention when administered to a living organism, such as, for example, a human. A pharmaceutically active metabolite can have a smaller, larger, or the same molecular weight as the corresponding compound of the invention from which it is derived. Non-limiting examples of metabolites are those substances resulting from in vivo degradation, oxidation, glycosylation, or isomerization.

The term “pharmaceutically acceptable prodrugs” as used herein, means prodrugs of the compounds of the present invention which are, within the scope of sound medical judgement, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “prodrug,” as used herein, represents compounds which are transformed in vivo into a parent compound of the above formula, for example, by hydrolysis in blood. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996), each of which is incorporated herein by reference.

By “ring system substituent” is meant a substituent attached to an aromatic or non-aromatic ring system. When a ring system is saturated or partially saturated the “ring system substituent” further includes methylene (double bonded carbon), oxo (double bonded oxygen) or thioxo (double bonded sulfur).

The term “spiroalkyl,” as used herein, represents an alkylene diradical, both ends of which are bonded to the same carbon atom of the parent group to form a spirocyclic group.

The term “sulfonyl,” as used herein, represents an —S(O)₂— group.

The term “thioalkoxy,” as used herein, represents represents an alkyl group attached to the parent molecular group through a sulfur atom. Exemplary unsubstituted thioalkoxy groups are of from 1 to 6 carbons.

The term “thioalkoxyalkyl,” as used herein, represents an alkyl group substituted by a thioalkoxy group. Exemplary unsubstituted thioalkoxyalkyl groups are of from 2 to 12 carbons.

By “thiocarbonyl” is meant a —C(S) group.

By “thiol” is meant an —SH group.

Asymmetric or chiral centers may exist in the compounds of the present invention. The present invention contemplates the various stercoisomers and mixtures thereof. Individual stereoisomers of compounds or the present invention are prepared synthetically from commercially available starting materials which contain asymmetric or chiral centers or by preparation of mixtures of enantiometic compounds followed by resolution well-known to those of ordinary skill in the art. These methods of resolution are exemplified by (1) attachment of a racemic mixture of enantiomers, designated (+/−), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R” or “S,” depending on the configuration of substituents around the chiral carbon atom.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration, where the term “Z” represents substituents on the same side of the carbon-carbon double bond and the term “E” represents substituents on opposite sides of the carbon-carbon double bond. It is also recognized that for structures in which tautomeric forms are possible, the description of one tautomeric form is equivalent to the description of both, unless otherwise specified. For example, amidine structures of the formula —C(═NR^{Q})NHR^{T} and —C(NHR^{Q})═NR^{T}, where R^{T} and R^{Q} are different, are equivalent tautomeric structures and the description of one inherently includes the other.

### BRIEF DESCRIPTION OF THE DRAWINGS

FIG. 1 is a simplified diagrammatic representation of the coagulation cascade showing the role of Factor XI in this pathway. All three thick arrows represent the amplification phase. The initiation and propagation phases are also labeled. FIIa is also known as thrombin. The arrow from FIIa to FXIa indicates that FXI is activated by FIIa and FXIIa.

FIG. 2 is a synthetic scheme showing the steps in the preparation of compounds of formula XI, including procedures A, B, C, D, E, F, G, H, and I.

FIG. 3 is a synthetic scheme showing the steps in the preparation of compounds of formula XVII, including procedures C, D, E, F, G, H, I, and J.

FIG. 4 is a synthetic scheme showing the steps in the preparation of compounds of formula XIX, including procedures C, D, E, G, H, I, and K.

FIG. 5 is a synthetic scheme showing the steps in the preparation of compounds of formulas XXII and XXVI, including procedures B, C, D, E, G, H, I, and L.

FIG. 6 is a synthetic scheme showing the steps in the preparation of compounds of formulas XXIIa and XXIIIa, including procedures B, G, H, I, and L.

FIG. 7 is a synthetic scheme showing the steps in the preparation of compounds of formula XXX, including procedures C, D, E, M, N, and O.

FIG. 8 is a synthetic scheme showing the steps in the preparation of compounds of formulas XXXII, XXXIII, XXXIV, XXXV, XXXVI, and XXXVII, including procedures B, C, D, E, F, G, H, I, L, P, Q, R, S, and T.

FIG. 9 is a scheme showing the use of the palladium-mediated cross couplings of Procedure U or Procedure V in the synthesis of intermediates useful in the preparation of compounds of the invention.

FIG. 10 is a scheme showing the use of the palladium-mediated cross couplings of Procedure W in the synthesis of intermediates useful in the preparation of compounds of the invention.

FIG. 11 is a synthetic scheme showing the steps in the preparation of compounds of formulas LXII and LXV, including procedures C, D, E, F, G, H, I, Y, X, and Z.

### DETAILED DESCRIPTION

### General Synthetic Methods for the Compounds of the Present Invention

### General Alkylation Procedure A

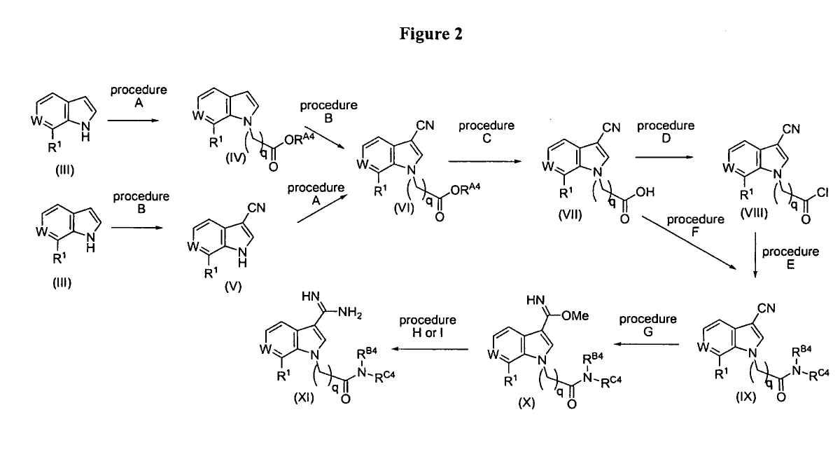

As shown in FIG. 2, an indole compound (W is CH) or a pyrrolopyridine compound (W is N) of formula III is added to a suspension of NaH (960 mg, 60% in mineral oil, 24 mmol) in DMF (20 mL) under ice-bath. The resultant slurry is stirred at RT for 30 min, then cooled down to 0° C., to this mixture was added an alkyl halide (24 mmol), such as, for example, ethyl bromoacetate (q is 1) or ethyl bromopropionate (q is 2). The reaction mixture is warmed to RT and then heated to 45° C. for 1.5 h. After cooling to room temperature, the reaction mixture is diluted with ethyl acetate (200 mL) and washed with brine (3×30 mL). The organic layer is dried over Na₂SO₄ and concentrated under vacuum to provide a compound of formula IV, which can be purified by silica gel chromatography (0-25% ethyl acetate in hexane gradient). Similarly, a compound in which R⁴ is —(CH₂)_{q}CO₂R^{A4}, where q is 1-6, can be prepared using the appropriate bromoalkylenate, Br—(CH₂)_{q}CO₂R^{A4}, where each of R¹ and R^{A4} is as previously defined.

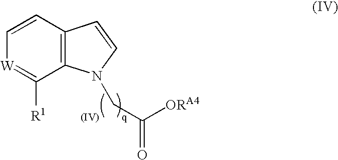

### General Cyanation Procedure B

As shown in FIG. 2, an indole compound (W is CH) or a pyrrolopyridine compound (W is N) of formula III in dry DCM is treated portionwise with chlorosulfonyl isocyanate 1.0 eq.) over 30 minutes at 0° C. (see Tetrahedron 50(22), 6549-58; 1994). After stirring for 1 hour at 0° C., a solution of triethylamine (0.98 eq) in acetonitrile is added dropwise within 45 min at 0-2° C., and then the reaction was warmed to RT and stirred for another 2 h. After removal of the volatiles, the residue is taken up in chloroform and ice-cooled saturated NaHCO₃ solution. The aqueous layer is washed with additional cold chloroform (2×) and the combined chloroform solutions are dried over Na₂SO₄, filtered, and concentrated in vacuo. The residue is purified by silica gel chromatograpy to give a compound of formula V, which can be subsequently converted to a compound of formula VI using general alkylation procedure A.

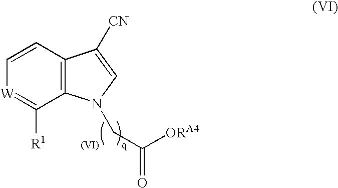

### General Saponification Procedure C

As shown in FIG. 2, a compound of formula VI in THF is treated with 1M NaOH (equal volume to THF, 2.5 eq.), which is added dropwise. The resultant reaction mixture is stirred at RT for 1 h and then acidified to pH 3.7-pH 4.3. A precipitate is collected as product and the filtrate extracted with ethyl acetate. The organic layer is dried over Na₂SO₄ and concentrated and the resulting solid combined with that previously collected by filtration to provide a compound of formula VII as product.

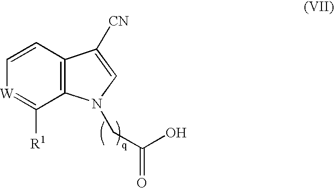

### General Procedure D for the Formation of Acyl Chlorides

As shown in FIG. 2, a compound of formula VIII can be prepared as follows. Oxalyl chloride (5 eq.) is added to the solution of a compound of formula VII in DCM followed by addition of one drop of DMF. The resultant mixture is stirred at RT for 1 h, followed by concentration of the reaction mixture under vacuum. The residue is re-dissolved in DCM followed by removal of the volatiles under vacuum, which is repeated once to ensure removal of excess oxalyl chloride. The resulting acyl chloride of formula VIII can be used directly in subsequent reactions without further purification.

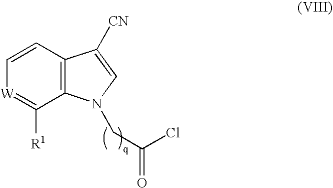

General Procedure E for the Formation of Amides via Acyl Chlorides

As shown in FIG. 2, a compound of formula VIII is dissolved in DCM and transferred to a suspension containing an amine (H—NR^{B4}R^{C4}, 1.1 eq.) and K₂CO₃ (3 eq.) in THF (equal volume to that of DCM used previously) under vigorous stirring. The reaction mixture is stirred for 1.5 h at ambient temperature. The mixture was filtered and the filtrate was concentrated under vacuum and then dissolved in ethyl acetate. The ethyl acetate solution is washed with water, brine, dried over Na₂SO₄, and concentrated under vacuum to afford a compound of formula IX, where each of R¹, R^{B4}, and R^{C4} is as previously defined. An analytical sample can be obtained by silica gel chromatography, using either hexanes/ethyl acetate or DCM/methanol (95:5, v/v) as eluant system.

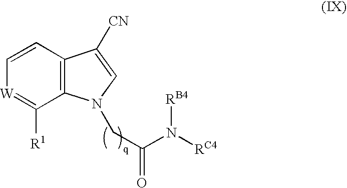

### General Procedure F for the Formation of Amides via Carbodiimide-Mediated Carboxylic Acid Activation

As shown in FIG. 2, to a suspension containing a compound of formula VII (1.0 eq.), HOBt (1.2 eq.), EDC (1.2 eq.), and DIEA (3 eq.) is added an amine (H—NR^{A4}R^{B4}, 1.1 eq.). The reaction mixture is stirred at ambient temperature for 1.5 h and concentrated under vacuum. The residue is taken up in ethyl acetate followed by washing with 5% HOAc, sat. NaHCO₃ and brine successively. The organic layer is dried over Na₂SO₄, filtered, and concentrated to provide a compound of formula IX, where each of R¹, R^{B4}, and R^{C4} is as previously defined, and which can be purified as previously described.

### General Procedure G for the Formation of Imidates

As shown in FIG. 2, an imidate of formula X can be prepared from a compound of formula IX by using a modified literature procedure (Wendt, et al., J. Med. Chem. 47:303-324, 2004). Accordingly, a 6N HCl in methanol solution is prepared by adding acetyl chloride to methanol (2:3, v/v) slowly at 0° C., followed by stirring at ambient temperature for 30 min. The compound of formula IX is added and the reaction mixture is stirred at RT, while monitoring the progress of the reaction by LC-MS analysis. When conversion of the nitrile to the imidate is complete (normally 1-6 hours), the reaction mixture is concentrated under vacuum and the resulting residue used directly in one of the amidine-forming reactions of general procedures H or I.

### General Procedure H for the Formation of Amidines

As shown in FIG. 2, a compound of formula X is dissolved in methanol, treated with ammonium acetate (anhydrous, 6-10 eq.), and stirred at rt for 24 h. The progress of the reaction is monitored by LC-MS analysis until the reaction is judged to be complete. At this time, the reaction mixture is concentrated under vacuum to provide a crude amidine product, which can be purified by preparative HPLC using acetonitrile-water (containing 0.1% TFA as modifier) gradient system to provide amidines, such as a compound of formula XI.

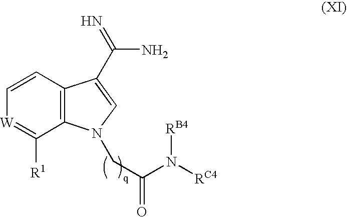

### General Procedure I for the Formation of Amidines

The general procedure I for the preparation of an amidine of formula XI from the corresponding nitrile is similar to procedure H, except that a 7N ammonia in methanol solution is used instead of ammonium acetate for the conversion of imidates to amidines.

### Examples Using Procedures A Through I

Several examples in which compounds of the invention are prepared by any of the procedures A through I are as follows. In one example, compound IXa, in which X⁴ is CH or N and R^{Q4} is carbomethoxy, is prepared starting from a compound of formula III in which W is CH and R¹ is CH₂═CHCH₂OC(O)— via procedures A, B, C, D, and E, using ethyl bromoacetate in procedure A and the appropriately substituted aniline or aminopyridine compound in step E.

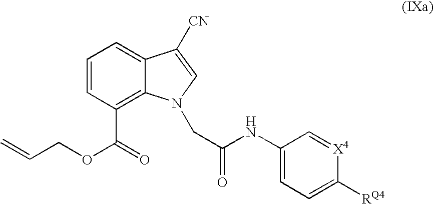

The allyl protecting group is subsequently catalytically removed and the resulting carboxylic acid is coupled to an appropriately substituted benzyl amine using procedures D and E to produce a compound of formula IXb, in which X⁴ is CH or N, R^{Q4} is carbomethoxy, R^{AA1} is hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and R^{G1} is substituted or unsubstituted C₆ or C₁₀ aryl.

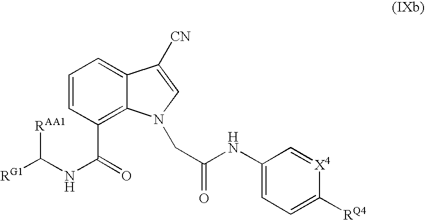

Pinner reaction, as exemplified by procedures G and H, followed by saponification of the R^{Q4} carbomethoxy group, produces a compound of formula IXc, where X⁴, R^{G1}, and R^{AA1} are as defined above.

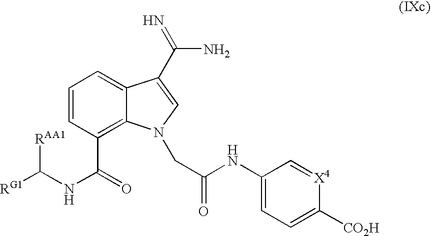

In another example, a compound of formula IV in which R¹ is CHO, q is 1, W is CH and R^{A4} is ethyl, is treated with 3-aminopyridine in a reductive amination procedure to produce compound IXd, which is subsequently subjected to procedures B, D, E (using 4-amino-methylbenzoate as the amine component) G, and H to produce compound IXe, which is subsequently saponified to produce compound IXf.

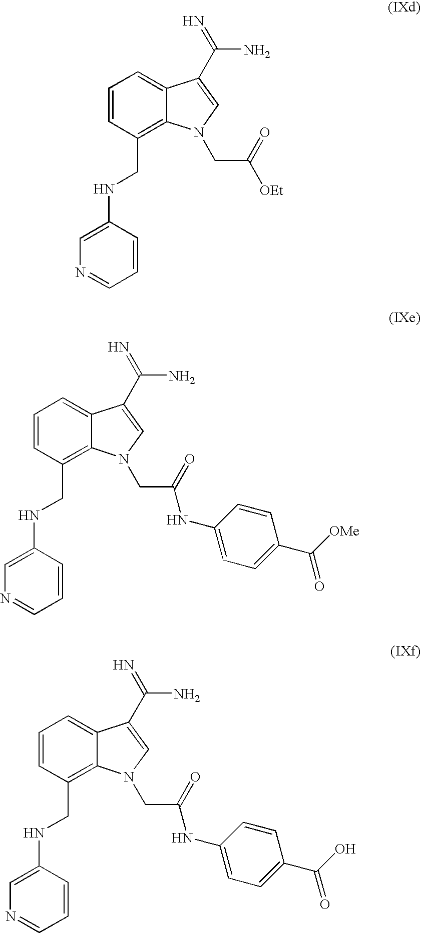

In yet another example, the compound of formula VI in which W is CH, R¹ is CO₂H, q is 1, and R^{A4} is CH₂CH₃ is coupled to a sulfonamide using a carbodiimide coupling reagent, such as EDCI, in the presence of a catalytic amount of 4-dimethylaminopyridine (see Matassa et al., J. Med. Chem. 33:1781-1790, 1990) to produce a compound of formula IXg, in which R^{G1} is a substituted or unsubstituted C₆ or C₁₀ aryl. This compound can be treated with base to deprotonate the sulfonamide nitrogen, followed by reaction with an alkylating agent, such as an alkyl halide, to produce a compound of formula IXh, in which R^{G1} is defined as above, nn is an integer of from 1 to 4, and X^{1a} is CH or N.

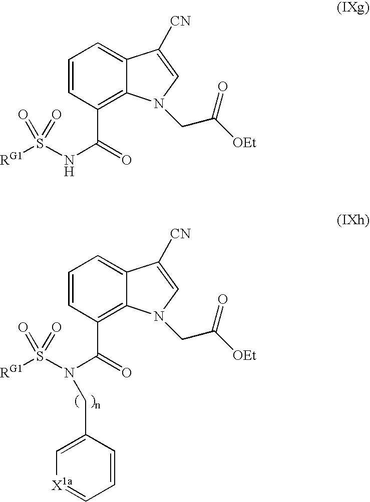

A compound of formula IXh can be subjected to procedures B, D, E (using 4-amino-methylbenzoate as the amine component) G, H, and C to produce a compound of formula IXi, in which R^{G1}, nn, and X^{1a} are as described above.

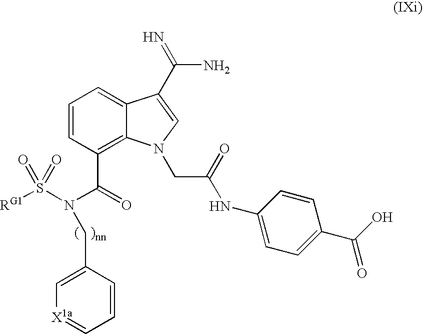

General Procedure J for the N-Alkylation of the Indole Amine with Acetylenes

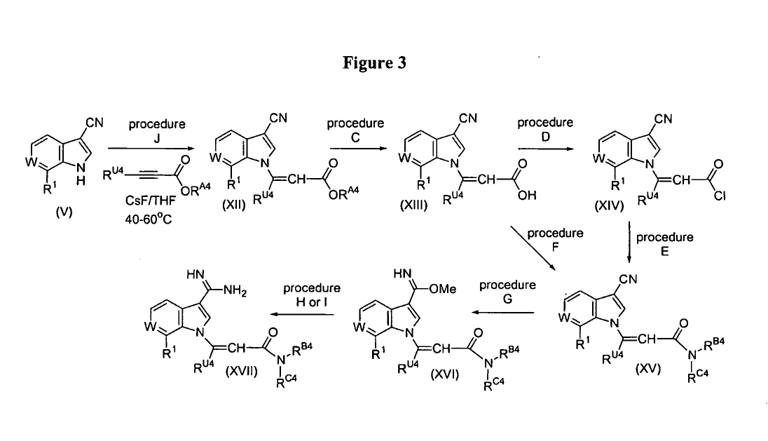

As shown in FIG. 3, a compound of formula V (3.52 mmol) and an acetylenyl compound containing an electron-withdrawing group, such as, for example, R^{U4}—C≡C—CO₂R^{A4} (4.22 mmol) are dissolved in 10 mL THF in the presence of a weak base, such as, for example, CsF or tetrabutylammonium fluoride (7.0 mmol). The reaction mixture is stirred at 23° C.-60° C. for several hours while monitoring the progress of the reaction by LC-MS analysis. When the reaction is complete, the reaction mixture is cooled, diluted with ethyl acetate (20 mL) and washed with water and brine. After drying over sodium sulfate, the organics are concentrated under vacuum to yield a compound of formula XII, which is a mixture of E and Z isomers. This compound can be purified by chromatography, thereby separating the isomers, or carried on to the next reaction as the mixture of isomers. A compound of formula XVII, where each of R¹, R^{U4}, R^{B4}, and R^{C4} is as previously defined, is produced from a compound of formula XII by the sequential application of procedures C, D, E, F, G, and H or I.

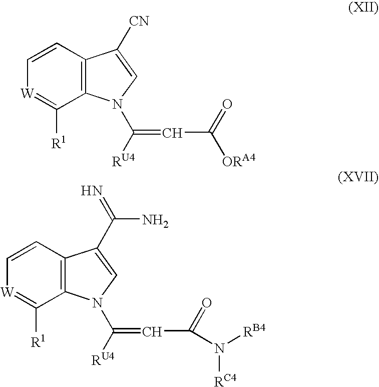

### General Procedure K for the Reduction of a Double Bond

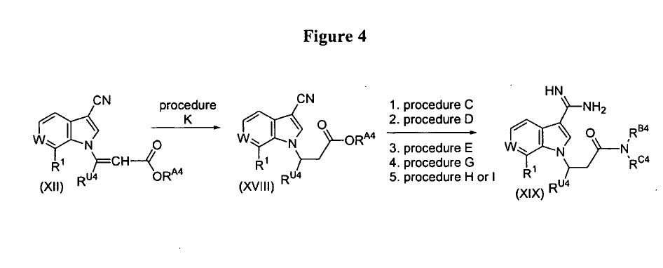

As shown in FIG. 4, a compound of formula XII (1.58 mmol) is dissolved in methanol (10 mL) and hydrogenated in the presence of 10% Pd/C (100 mg) overnight. The reaction mixture is filtered and concentrated under vacuum to yield a compound of formula XVIII. A compound of formula XIX, where each of R¹, R^{U4}, R^{B4}, and R^{C4} is as previously defined, is produced from a compound of formula XVIII by the sequential application of procedures C, D, E or F, G, and H or I.

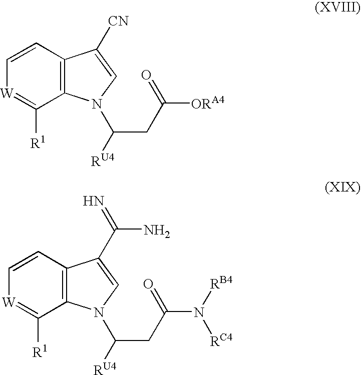

General Procedure L for the N-Arylation of an Indole Nitrogen with an Aryl Fluoride

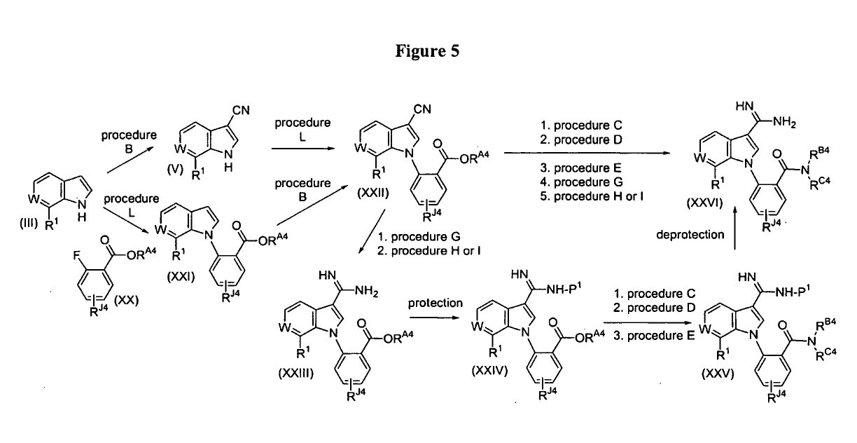

As shown in FIG. 5, a compound of formula XXII can be prepared by an arylation procedure that includes treatment of an indole, such as, for example, a compound of formula V (1.0 eq.), with a base (such as, for example, Na₂CO₃, Cs₂CO₃, KOtBu, or NaH; 1.5 eq.), followed by reaction with a fluorophenyl compound having an electron-withdrawing group, such as, for example, a compound of formula XX (1.1 eq.). In one example, 3-cyanoindole (3.9 mmol) was dissolved in dry DMF (17 mL), followed by addition of 5.9 mmol of sodium hydride as a 60% mineral oil dispersion. After the mixture is stirred for 30 minutes at room temperature, a solution of 4-bromo-2-fluoro-benzoic acid methyl ester (4.30 mmol) in 3 mL DMF was added. The reaction mixture was stirred at 80° C. for 3 hours, followed by cooling to room temperature and treatment with a saturated NH₄Cl solution (80 mL). The mixture was extracted with ethyl acetate (3×80 mL) and the combined organics washed with brine and dried over sodium sulfate. After filtration and concentration under vacuum, the product was purified by chromatography to give 4-bromo-2-(3-cyano-indol-1-yl)benzoic acid, methyl ester.

A compound of formula XXII can be transformed into an amidine of formula XXVI, where each of R¹, R^{J4}, R^{B4}, and R^{C4} is as previously defined, by the sequential application of procedures C, D, E or F, G, and H or I. Alternatively, the nitrile moiety of a compound of formula XXII can be converted to an amidine of formula XXIII, where each of R¹, R^{J4}, and R^{A4} is as previously defined, by procedures G and H or I.

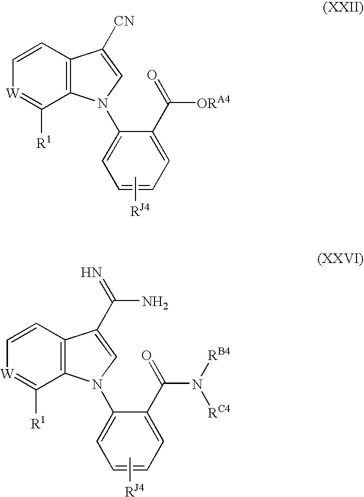

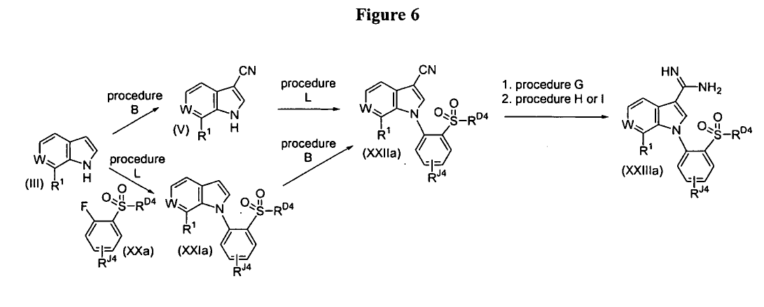

Similarly, as shown in FIG. 6, a compound of formula XXIIa can be prepared by an arylation procedure that includes treatment of an indole, such as, for example, a compound of formula V (1.0 eq.), with a base (such as, for example, Na₂CO₃, Cs₂CO₃, KOtBu, or NaH; 1.5 eq.), followed by reaction with a fluorophenyl compound having an electron-withdrawing group, such as, for example, a compound of formula XXa (1.1 eq.). Transformation of the cyano group of compounds of formula XXa to the amidino group of compounds of formula XXIIIa can be accomplished as previously described using procedure G, followed by procedure H or I.

### General Procedure for the Protection/Deprotection of Amidines Via Carbamates

Procedures for the protection of amidines are known to those skilled in the art (see the discussion of amine protection in Greene, “Protective Groups In Organic Synthesis, 3^{rd} Edition” (John Wiley & Sons, New York, 1999) or Bailey et al., Amidine protection for solution phase library synthesis. Tetrahedron Letters, 40:4847-4850, 1999). In one example, as shown in FIG. 5, a compound of formula XXIII (1 mmol) is dissolved in THF (10 mL) and di-t-butyldicarbonate (1.1 mmol) is added, followed by the addition of diisopropylethylamine (1.2 mmol). The reaction mixture is stirred at room temperature for 16 hours, followed by concentration under vacuum. The crude product is taken up in ethyl acetate (100 mL), washed with 5% citric acid, 5% sodium bicarbonate, water, brine, and dried over sodium sulfate. Filtration followed by removal of the votatiles under vacuum produced a compound of formula XXIV in which P¹ is —CO₂tBu (Boc) and each of R¹, R^{J4}, and R^{A4} is as previously defined. A compound of formula XXIV can be converted to a protected amidine of formula XXV by the sequential application of procedures C, D, and E. The compound of formula XXV can then be deprotected by treatment with 40% TFA/CH₂Cl₂ and purified by HPLC to produce an amidine of formula XXVI, where each of R¹, R^{J4}, R^{B4}, and R^{C4} is as previously defined. The amidine protecting group is chosen such that its deprotection is complementary with other functionality that may exist in the molecule. In addition to the Boc group described above, other amidine protecting groups include the Cbz protecting group, which can be removed via a hydrogenation procedure, and the trityl protecting group, which can be removed by treatment with very mild acid.

### General Procedure M for the Formation of Amidines via Isoxazoles

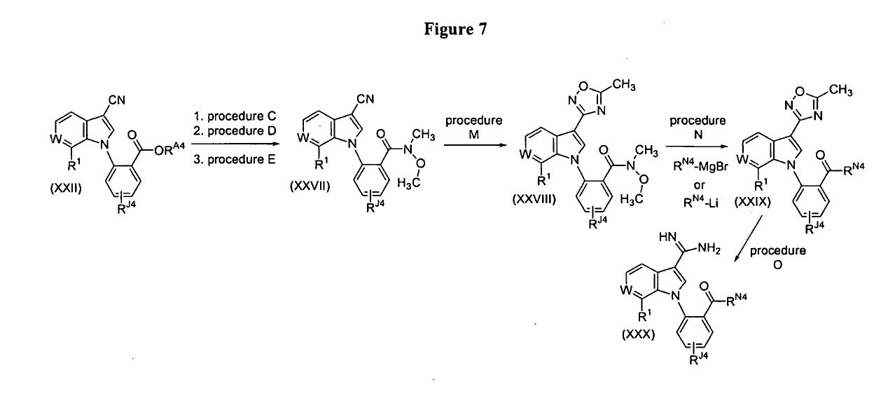

As shown in FIG. 7, a nitrile of formula XXVII (obtained from a compound of formula XXII by a sequence of reactions that includes procedures C, D, and E, with N-methoxy-N-methylamine used in procedure E) is reacted with hydroxylamine under elevated temperatures, followed by acetylation of the intermediate hydroxylaminoimidate with acetyl chloride and diisopropylethylamine, and then tetrabutylammonium fluoride-mediated cyclization to the 1,2,4-oxadiazole of formula XXVIII.

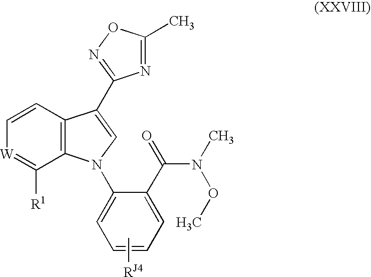

General Procedure N for the Reaction of Weinreb Amides with Organometallic Agents

As shown in FIG. 7, reaction of an N-methoxy-N-methylamide (Weinreb amides) of formula XXVIII with alkyl, alkenyl, cycloalkyl, aralkyl, aryl, heteroaryl, or heteroaralkyl carbanions such as lithium salts (R^{N4}—Li) or Grignard reagents (R^{N4}—MgBr) to form ketones of formula XXIX, where each of R¹, R^{J4}, and R^{N4} is as previously defined.

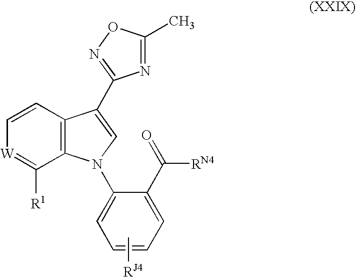

General Procedure O for the Conversion of 1,2,4-oxadiazoles to Amidines

As shown in FIG. 7, oxadiazoles of formula XXIX can be transformed into amidines of formula XXX, where each of R¹, R^{J4}, and R^{N4} is as previously defined, by either catalytic hydrogenation or by treatment with Fe powder.

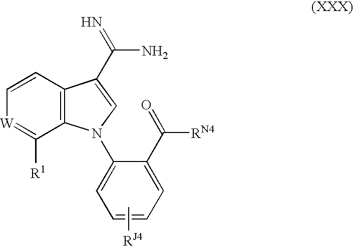

### General Procedure P for Converting Nitroaryl Compounds to Anilines

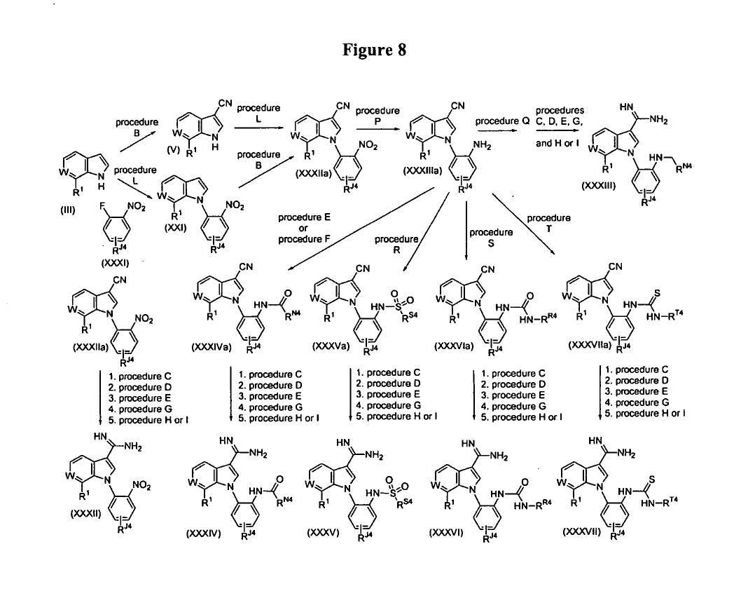

As shown in FIG. 8, a compound of formula XXXIIa (obtained from a compound of formula V by arylation of the indole nitrogen with a 2-nitrofluorobenzene of formula XXXI using general procedure L) can be converted to an aniline of formula XXXIIIa (1 mmol) via reduction of the nitro group using SnCl₂ (10 mmol) in 1:1 CH₂Cl₂/DMF. In one example, the nitro compound (1.0 mmol) is treated with tin(II) chloride dihydrate (5.0 mmol) in ethanol (12 mL) at 70° C. for 16 h. TLC analysis or LC-MS analysis indicates the completion of the reaction. The reaction mixture is concentrated to about half volume (˜5 mL), and poured into ice-cold water (30 mL). The pH is made slightly basic (pH=7-8) by addition of 5% NaHCO₃ before being extracted with ethyl acetate (30 mL×3). The combined organic layers are washed with brine (20 mL×3). The organic layer is treated with charcoal and filtered through celite. The filtrate is dried over Na₂SO₄, and concentrated to afford the desired aniline.

The compound of formula XXXIIa can also be reduced to an aniline of formula XXXIIIa by catalytic hydrogation (see general procedure K).

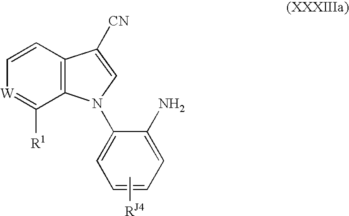

The aniline of formula XXXIIIa can be subsequently used to produce a nitrile of formula XXXIVa by reaction with an acyl chloride (such as, for example, R^{N4}—COCl) or a carboxylic acid (such as, for example, R^{A4}—COOH) by procedures E or F, respectively. The nitrile of XXXIVa can then be converted to an amidine of formula XXXIV, where each of R¹, R^{J4}, and R^{N4} is as previously defined, by the sequential application of procedures C, D, E, G, and H or I. In one example, the amine compound (1.0 mmol) is dissolved in anhydrous DCM (20 mL). Acid chloride (1.5 mmol) is added, followed by addition of triethylamine (1.7 mmol). The reaction mixture is shaken at room temperature overnight. WAJ21 resin (loading: 5 mmol/g, 0.5 mmol, Aldrich product) is used to scavenge the excessive acid chloride or sulfonyl chloride. The mixture is shaken at room temperature for 5-6 h. The resin is filtered and concentration of the filtrate gives the desired product.

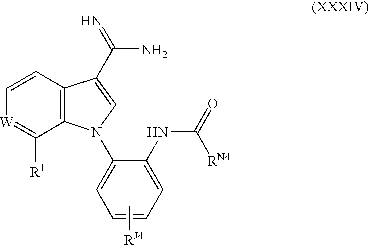

Alternatively, the nitro compound of formula XXXIIa can be converted to an amidine of formula XXXII, where each of R¹ and R^{J4} is as previously defined, by the sequential application of procedures C, D, E, G, and H or I.

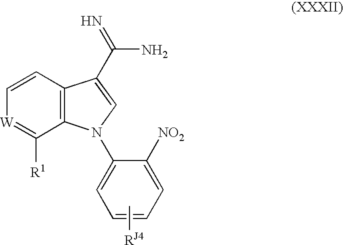

### General Procedure Q for the Alkylation of Anilines

As shown in FIG. 8, an aniline compound of formula XXXIIIa can by N-alkylated by treating the compound with a base (such as, for example, sodium hydride), followed by treatment with an alkylating agent (such as, for example, an alkyl halide). Alternatively, the aniline compound can be used as the amine component in a palladium catalyzed cross-coupling reaction, as described below in Procedure W. After alkylation, the intermediate nitrile is converted to an amidine of formula XXXIII by the sequential application of procedures C, D, E, G, and H or I.

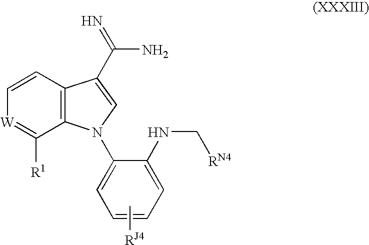

### General Procedure R for the Preparation of Sulfonamides

As shown in FIG. 8, a compound of formula XXXIIIa can be reacted with a sulfonyl chloride (such as, for example, R^{S4}—S(O)₂Cl) to produce a nitrile of formula XXXVa, which can then be converted to an amidine of formula XXXV, where each of R¹, R^{J4}, and R^{S4} is as previously defined, by the sequential application of procedures C, D, E, G, and H or I. In one example, the amine compound (1.0 mmol) is dissolved in anhydrous DCM (20 mL). Sulfonyl chloride (1.5 mmol) is added, followed by addition of triethylamine (1.7 mmol). The reaction mixture is shaken at room temperature overnight. WAJ21 resin (loading: 5 mmol/g, 0.5 mmol, Aldrich product) is used to scavenge the excessive acid chloride or sulfonyl chloride. The mixture is shaken at room temperature for 5-6 h. The resin is filtered and concentration of the filtrate gives the desired product.

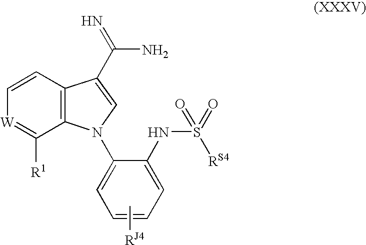

General Procedure S for the Preparation of Ureas

As shown in FIG. 8, a compound of formula XXXIIIa can be first reacted with phosgene, or a phosgene equivalent, followed by reaction with an amine (such as, for example R^{R4}—NH₂) to produce a compound of formula XXXVIa, which can then be converted to an amidine of formula XXXVI, where each of R¹, R^{J4}, and R^{R4} is as previously defined, by the sequential application of procedures C, D, E, G, and H or I. In one example, the amine compound (1.0 mmol) is dissolved in anhydrous DCM (20 mL), isocyanate (1.5 mmol) is added, followed by addition of a catalytic amount of solid-support DMAP resin (0.5 mmol). The reaction mixture is shaken at room temperature overnight. Trisamine resin (loading: 1.48 mmol/g, 0.2 mmol) is used to scavenge the excessive isocyanate reagent. The mixture is shaken at room temperature for 5˜6 h. The resin was filtered and concentration of the filtrate gives the desired product.

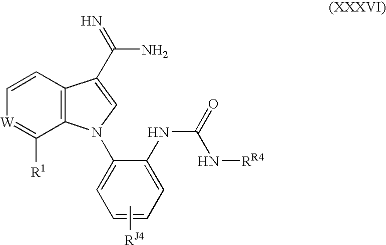

### General Procedure T for the Preparation of Thioureas

As shown in FIG. 8, a compound of formula XXXIIIa can be reacted with an isothiocyanate (such as, for example, R^{T4}—N═C═S), to produce a nitrile of formula XXXVIIa, which can then be converted to an amidine of formula XXXVII, where each of R¹, R^{J4}, and R^{T4} is as previously defined, by the sequential application of procedures C, D, E, G, and H or I. In one example, the amine compound (1.0 mmol) is dissolved in anhydrous DCM (20 mL), isothiocyanate (1.5 mmol) is added, followed by addition of a catalytic amount of solid-support DMAP resin (0.5 mmol). The reaction mixture is shaken at room temperature overnight. Trisamine resin (loading: 1.48 mmol/g, 0.2 mmol) is used to scavenge the excessive isocyanate reagent. The mixture is shaken at room temperature for 5-6 h. The resin is filtered and concentration of the filtrate gives the desired product.

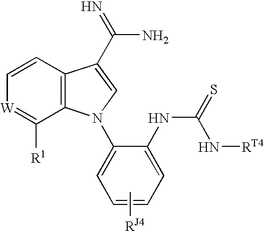

### General Procedure U for Palladium-Mediated Cross-Coupling Reactions

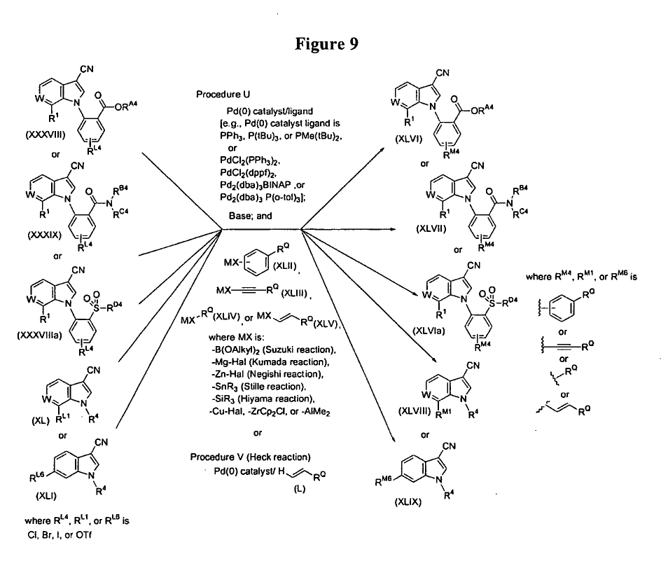

As shown in FIG. 9, a compound of formula XXXVIII, XXXVIIIa, XXXIX, XL, or XLI, that contains a leaving group that can be, for example, a halogen or a triflate, can be reacted with a palladium catalyst/ligand system (such as, for example, Pd(PPh₃)₄, Pd(PtBu₃)₄, Pd[P(Me)(tBu₂)]₄, PdCl₂(PPh₃)₂, PdCl₂(dppf)₂, Pd₂(dba)₃BINAP, or Pd₂(dba)₃P(o-tol)₃) in the presence of a base and an organometallic compound, such as for example, a compound of formula XLII, XLIII, XLIV, or XLV, where the MX moiety is —B(OH)₂ or —B(OAlkyl)₂ (Suzuki reaction), —Mg-Hal (Kumada reaction), —Zn-Hal (Negishi reaction), —Sn(Alkyl)₃ (Stille reaction), —Si(Alkyl)₃ (Hiyama reaction), —Cu-Hal, —ZrCp₂Cl, or —AlMe₂ to produce a compound of formula XLVI, XLVIa, XLVII, XLVIII, or XLIX, where R^{M4}, R^{M1}, or R^{M6} is —C₆H₄—R^{Q}, —C≡C—R^{Q}, CH═CH—R^{Q}, or —R^{Q}, where R^{Q} is hydrogen, substituted or unsubstituted C₁₋₆ alkyl, substituted or unsubstituted C₃₋₈ cycloalkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, substituted or unsubstituted heterocyclylalkyl, where the alkylene group is of one to six carbon atoms, substituted or unsubstituted C₂₋₇ alkanoyl, substituted or unsubstituted C₁₋₆ alkoxy, substituted or unsubstituted alkoxyalkyl, where the alkyl and alkylene groups are independently of one to six carbon atoms, substituted or unsubstituted C₁₋₆ alkylsulfinyl, substituted or unsubstituted alkylsulfinylalkyl, where the alkyl and alkylene groups are independently of one to six carbon atoms, substituted or unsubstituted C₁₋₆ alkylsulfonyl, substituted or unsubstituted alkylsulfonylalkyl, where the alkyl and alkylene groups are independently of one to six carbon atoms; substituted or unsubstituted C₇ or C₁₁ aryloyl, azido, halo, substituted or unsubstituted C₂₋₉(heterocyclyl)oxy, substituted or unsubstituted C₃₋₁₀(heterocyclyl)oyl, nitro, substituted or unsubstituted C₁₋₆ thioalkoxy, substituted or unsubstituted C₂₋₁₂ thioalkoxyalkyl, where the alkyl and alkylene groups are independently of one to six carbon atoms, —(CH₂)_{qq}CO₂R^{QA}, where qq is an integer of from zero to four and R^{QA} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, —(CH₂)_{qq}CONR^{QB}R^{QC}, where qq is an integer of from zero to four and R^{QB} and R^{QC} are independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms; and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N6}, where R^{N6} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{QA}, where R^{QA} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) —CONR^{QB}R^{QC}, where each of R^{QB} and R^{QC} is, independently, selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, and substituted or unsubstituted C₂₋₁₅ heterocyclylalkyl, where the alkylene group is of one to six carbon atoms, or R^{QB} taken together with R^{QC} and N forms a substituted or unsubstituted 5- or 6-membered ring, and (o) —S(O)₂R^{QS}, where R^{QS} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, with the proviso that no two groups are bound to the nitrogen atom through a carbonyl group or a sulfonyl group, —(CH₂)_{qq}S(O)₂R^{QD}, where qq is an integer of from zero to four and R^{QD} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, —(CH₂)_{qq}S(O)₂NR^{QE}R^{QF}, where qq is an integer of from zero to four and each of R^{QE} and R^{QF} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{QE} taken together with R^{QF} and N forms a substituted or unsubstituted 5- or 6-membered ring, —(CH₂)_{qq}NR^{QG}R^{QH}, where qq is an integer of from zero to four and each of R^{QG} and R^{QH} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{QN}, where R^{QN} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{QA}, where R^{QA} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (n) —S(O)₂R^{QS}, where R^{QS} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, with the proviso that no two groups are bound to the nitrogen atom through a carbonyl group or a sulfonyl group, perfluoroalkyl, perfluoroalkoxy, substituted or unsubstituted C₆ or C₁₀ aryloxy, and substituted or unsubstituted C₇₋₁₆ arylalkoxy, or —NR^{Qb}CONR^{QE}R^{QF}, where R^{Qb} is hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and each of R^{QE} and R^{QF} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C¹⁰ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{QE} taken together with R^{QF} and N forms a substituted or unsubstituted 5- or 6-membered ring. See Fu and Littke, Angew. Chem. Int. Ed. 41:4176-4211, 2002 for a review of palladium-catalyzed cross-coupling reactions.

In one example of a Suzuki reaction in which a boronic acids is couple to an aryl halide, a mixture of boronic acid (2 mmol), aryl halide (1 mmol), cesium carbonate (3 mmol) and bis(diphenylphosphinoferrocene)palladium(II) chloride (0.05 mmol) in DMF (10 mL) is heated to 100° C. overnight, or irradiated in a Microwave instrument at 100° C. for 20 min. The reaction mixture is cooled, quenched with water (20 mL) and extracted with ethyl acetate (2×10 mL). The organic layers are combined, dried and concentrated in vacuo. Purification by column chromatography gives the desired product.

In another example of a Suzuki reaction, a mixture of boronic acid (1.1 mmol), aryl halide (1.0 mmol), triethylamine (3 mmol) and bis(triphenylphosphine)palladium(II) chloride (0.05 mmol) in ethanol (30 mL) is irradiated in a Microwave instrument at 100° C. for 20 min. The reaction mixture is cooled and the solvent removed. The residue is treated with water (30 mL) and extracted with ethyl acetate (60 mL). The organic layer is dried and concentrated in vacuo. Purification by silica gel chromatography gives the desired product.

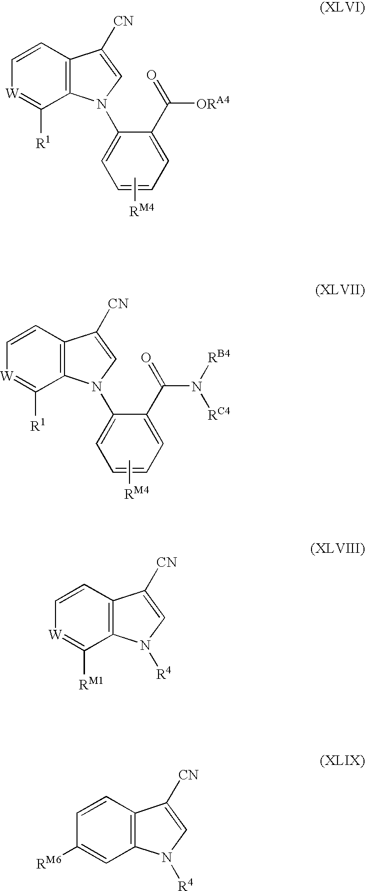

### General Procedure V for Palladium-Mediated Cross-Coupling Reactions

A compound of formula XXXVIII, XXXIX, XL, or XLI can be reacted with a palladium(0) catalyst in the presence of a base and an alkene, such as, for example, a compound of formula L, (see Heck, Palladium Reagents in Organic Synthesis, Academic Press, 1985) to produce a compound of formula XLVI, XLVII, XLVIII, or XLIX, where R^{M4}, R^{M1}, or R^{M6} is CH═CH—R^{Q}, where R^{A4}, R^{B4}, R^{C4}, and R^{Q} are as previously defined.

### General Procedure W for Palladium-Mediated Cross-Coupling Reactions

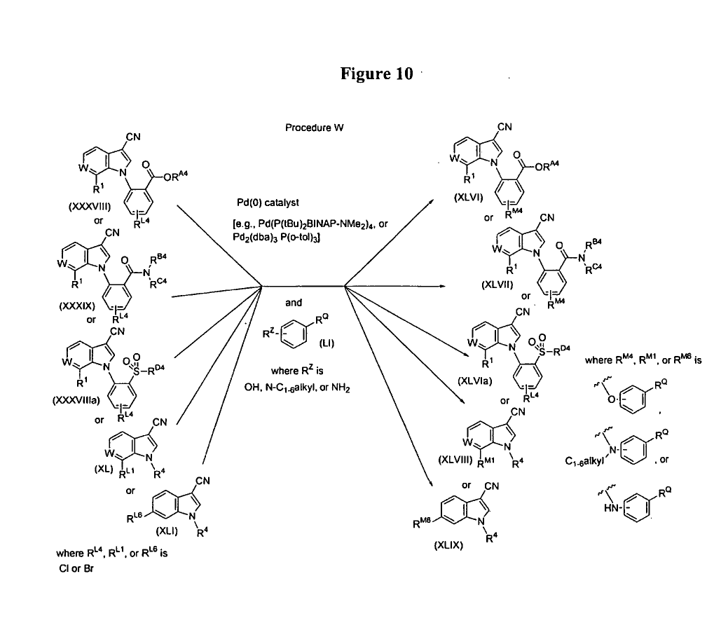

As shown in FIG. 10, a compound of formula XXXVIII, XXXVIIIa, XXXIX, XL, or XLI, that contains a leaving group that can be, for example, a chloro or a bromo, can be reacted with a palladium catalyst/ligand system (such as, for example, Pd(P(tBu)₂(dip-o-NMe₂))₄ or Pd₂(dba)₃P(o-tol)₃), see Fu and Littke, Angew. Chem. Int. Ed. 41:4176-4211, 2002) in the presence of a base and compound of formula LI, where R^{Z} is —OH, —NH—C₁₋₆alkyl, or —NH₂, to produce a compound of formula XLVI, XLVIa, XLVII, XLVIII, or XLIX, where R^{M4}, R^{M1}, or R^{M6} is —O—C₆H₄—R^{Q}, —NH—C₁₋₆alkyl-C₆H₄—R^{Q}, or —NH—C₆H₄—R^{Q}, where R^{A4}, R^{B4}, R^{C4}, and R^{Q} are as previously defined.

Table I includes compounds of formula XI prepared by the procedures described above.

TABLE I
Compounds of Formula XI

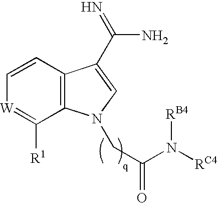

(XI), where q is one

| Cmpd | | | | |
|---|---|---|---|---|
| No. | R¹ | W | R^{B4} | R^{C4} |
| 1 | H | CH | H | 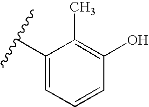 |
| 2 | H | CH | H | 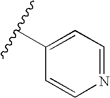 |
| 3 | H | CH | H | 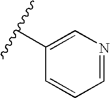 |
| 4 | H | CH | H | 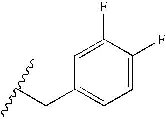 |
| 5 | H | CH | H | 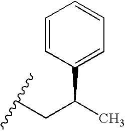 |
| 6 | H | CH | H | 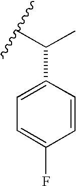 |
| 7 | H | CH | H | 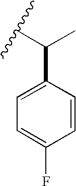 |
| 8 | Br | CH | H | 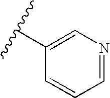 |
| 9 | H | CBr | H | 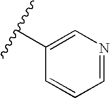 |
| 10 | Br | CH | H | 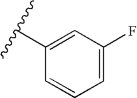 |
| 11 | Br | CH | H | 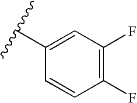 |
| 12 | Br | CH | H | 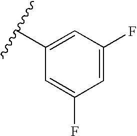 |
| 13 | Br | CH | H | 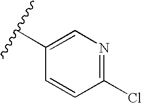 |
| 14 | Br | CH | H | 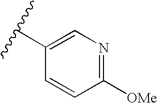 |
| 15 | Br | CH | H | 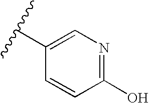 |
| 16 | Br | CH | H | 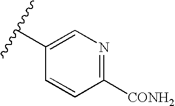 |
| 17 | Br | CH | H | NH₂ |
| 18 | Br | CH | H | 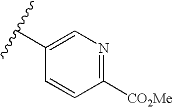 |
| 19 | Br | CH | H | 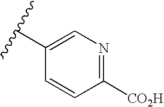 |
| 20 | Br | CH | H | 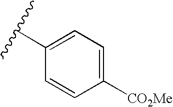 |
| 21 | Br | CH | H | 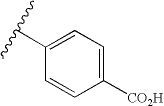 |
| 22 | Br | CH | H | 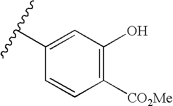 |
| 23 | Br | CH | H | 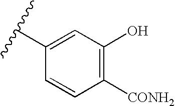 |
| 24 | Br | CH | H | 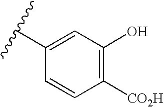 |
| 25 | Br | CH | H | 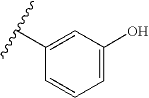 |
| 26 | Br | CH | H | 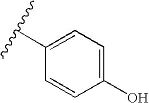 |
| 27 | Br | CH | H | 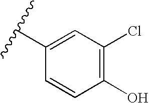 |
| 28 | Br | CH | H | 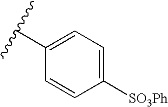 |
| 29 | Br | CH | H | 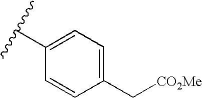 |
| 30 | Br | CH | H | 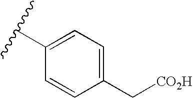 |
| 31 | Br | CH | H | 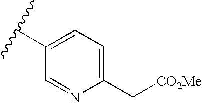 |
| 32 | Br | CH | H | 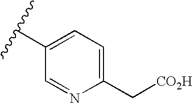 |
| 33 | Br | CH | H | 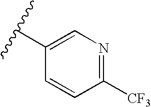 |
| 34 | Br | CH | H | 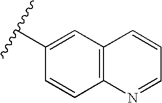 |
| 35 | Br | CH | H | 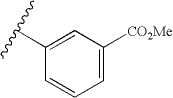 |
| 36 | Br | CH | H | 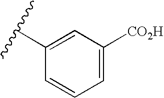 |
| 37 | Cl | N | H | 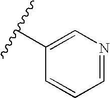 |
| 38 | Cl | N | H | 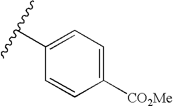 |
| 39 | Cl | N | H | 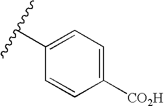 |
| 40 | H | 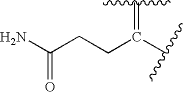 | H | 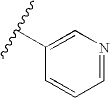 |
| 41 | H | 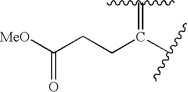 | H | 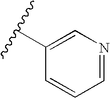 |
| 42 | H | 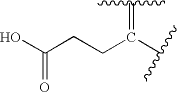 | H | 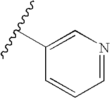 |
| 43 | 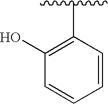 | CH | H | 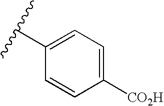 |
| 44 | 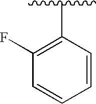 | CH | H | 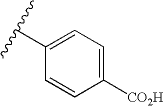 |
| 45 | 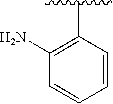 | CH | H | 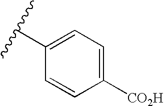 |
| 46 | 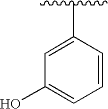 | CH | H | 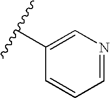 |
| 47 | 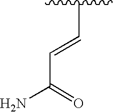 | CH | H | 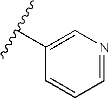 |
| 48 | 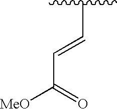 | CH | H | 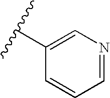 |
| 49 | 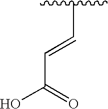 | CH | H | 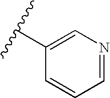 |
| 50 | 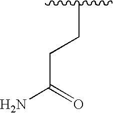 | CH | H | 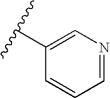 |
| 51 | 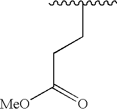 | CH | H | 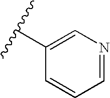 |
| 52 | 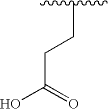 | CH | H | 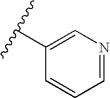 |
| 53 | 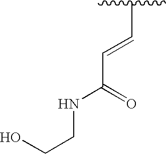 | CH | H | 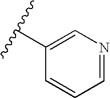 |
| 54 | 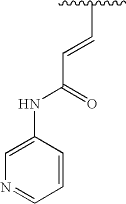 | CH | H | 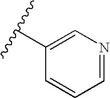 |
| 55 | 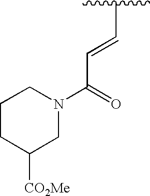 | CH | H | 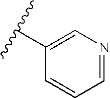 |
| 56 | 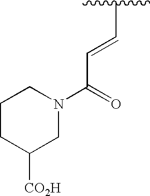 | CH | H | 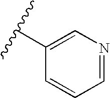 |
| 57 | 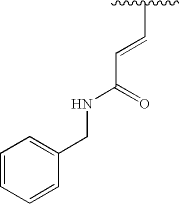 | CH | H | 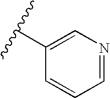 |
| 58 | 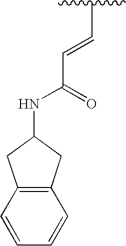 | CH | H | 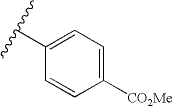 |
| 59 | 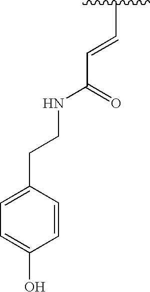 | CH | H | 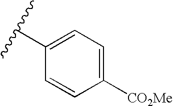 |
| 60 | 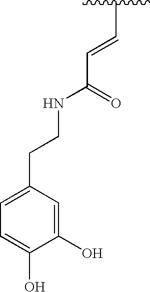 | CH | H | 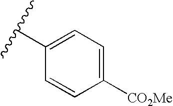 |
| 61 | 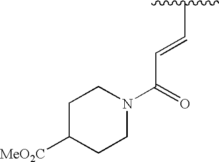 | CH | H | 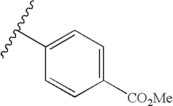 |
| 62 | 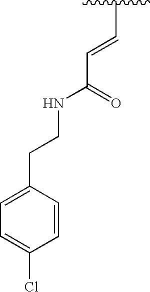 | CH | H | 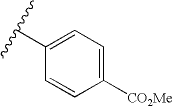 |
| 63 | 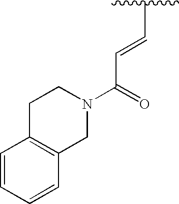 | CH | H | 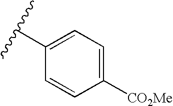 |
| 64 | 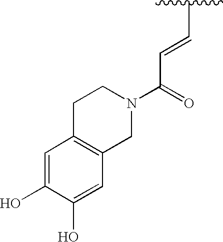 | CH | H | 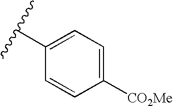 |
| 65 | 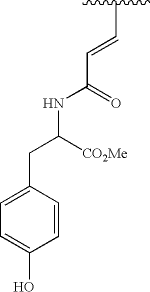 | CH | H | 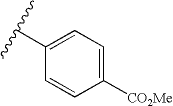 |
| 66 | 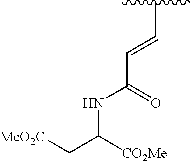 | CH | H | 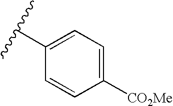 |
| 67 | 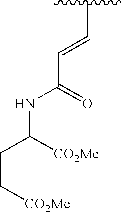 | CH | H | 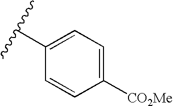 |
| 68 | 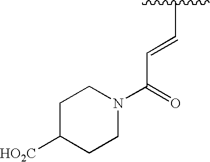 | CH | H | 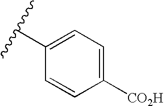 |
| 69 | 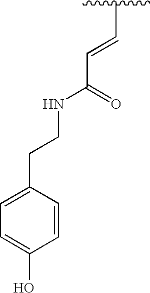 | CH | H | 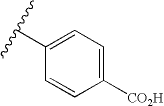 |
| 70 | 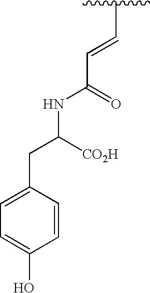 | CH | H | 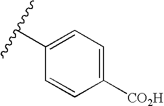 |
| 71 | 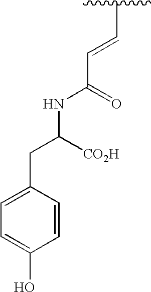 | CH | H | 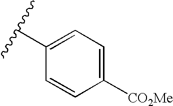 |
| 72 | 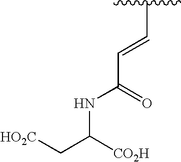 | CH | H | 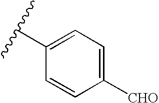 |
| 73 | 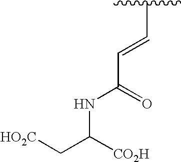 | CH | H | 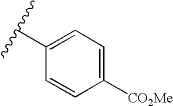 |
| 74 | 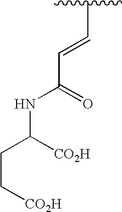 | CH | H | 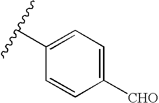 |
| 75 | 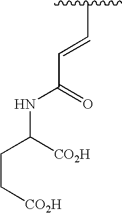 | CH | H | 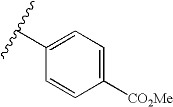 |
| 76 | 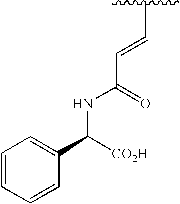 | CH | H | 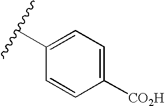 |
| 77 | 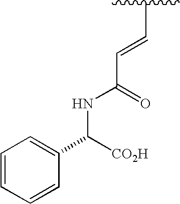 | CH | H | 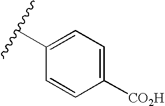 |
| 78 | 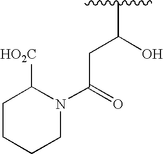 | CH | H | 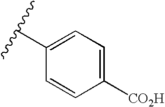 |
| 79 | 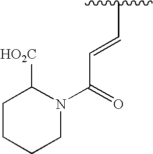 | CH | H | 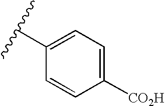 |
| 80 | 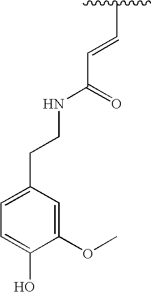 | CH | H | 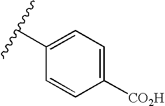 |
| 81 | 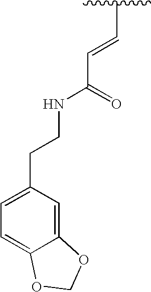 | CH | H | 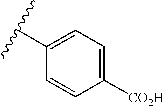 |
| 82 | 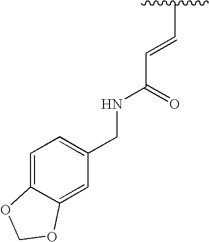 | CH | H | 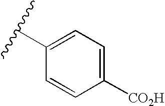 |
| 83 | 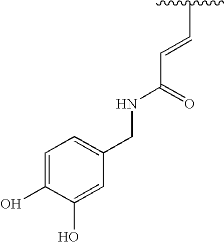 | CH | H | 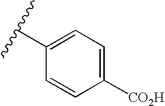 |
| 84 | 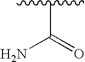 | CH | H | 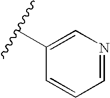 |
| 85 | 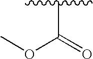 | CH | H | 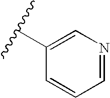 |
| 86 | 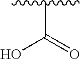 | CH | H | 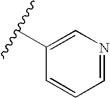 |
| 87 | 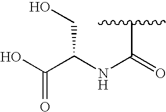 | CH | H | 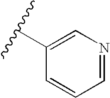 |
| 88 | 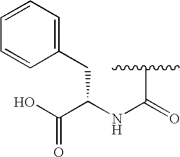 | CH | H | 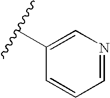 |
| 89 | 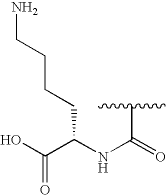 | CH | H | 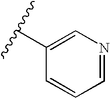 |
| 90 | 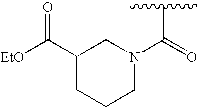 | CH | H | 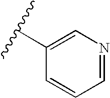 |
| 91 | 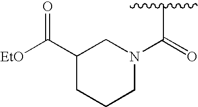 | CH | 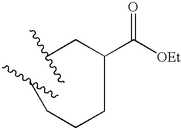 | |
| 92 | 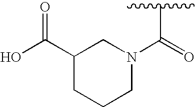 | CH | H | 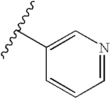 |
| 93 | 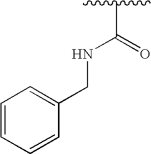 | CH | H | 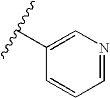 |
| 94 | 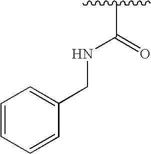 | CH | H | 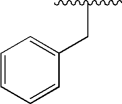 |
| 95 | 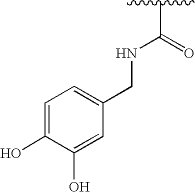 | CH | H | 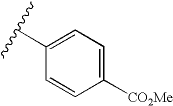 |
| 96 | 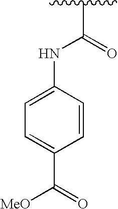 | CH | H | 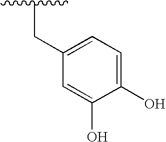 |
| 97 | 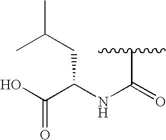 | CH | H | 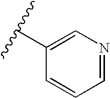 |
| 98 | 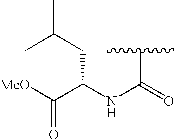 | CH | H | 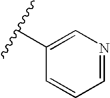 |
| 99 | 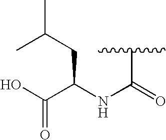 | CH | H | 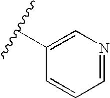 |
| 100 | 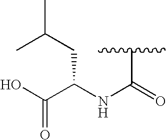 | CH | H | 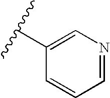 |

Table II includes compounds of formula XVII prepared by the procedures described above.

TABLE II
Compounds of Formula XVII

| | |
|---|---|
| | (XVII) |
| 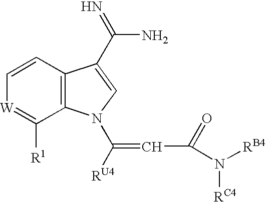 | |

| Cmpd | | | | | |
|---|---|---|---|---|---|
| No. | R¹ | W | R^{U4} | R^{B4} | R^{C4} |
| 101 | H | CH | 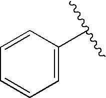 | 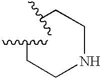 | |
| 102 | H | CH | 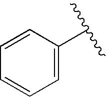 | 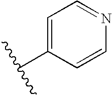 | H |
| 103 | H | CH | 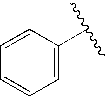 | 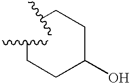 | |
| 104 | H | CH | 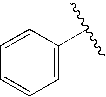 | 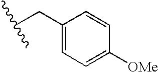 | H |
| 105 | H | CH | 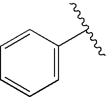 | 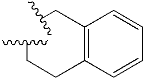 | |
| 106 | H | CH | 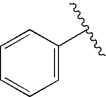 | 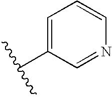 | H |
| 107 | H | CH | 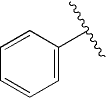 | 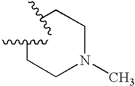 | |
| 108 | H | CH | 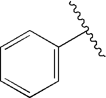 | 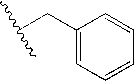 | H |
| 109 | H | CH | 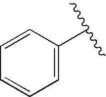 | 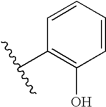 | H |
| 110 | H | CH | 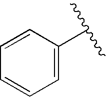 | 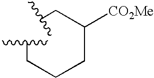 | |
| 111 | H | CH | 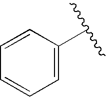 | 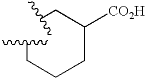 | |
| 112 | H | CH | 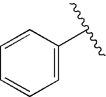 | 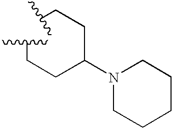 | |
| 113 | H | CH | 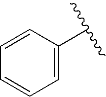 | 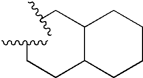 | |
| 114 | H | CH | 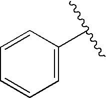 | 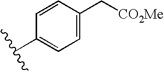 | H |
| 115 | H | CH | 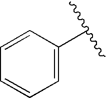 | 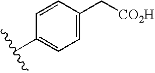 | H |

Table III includes compounds of formula XIX prepared by the procedures described above.

TABLE III
Compounds of Formula XIX

| | |
|---|---|
| | (XIX) |
| 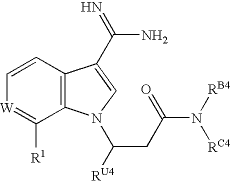 | |

| Cmpd | | | | | |
|---|---|---|---|---|---|
| No. | R¹ | W | R^{U4} | R^{B4} | R^{C4} |
| 116 | H | CH | 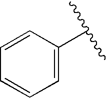 | 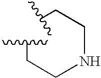 | |
| 117 | H | CH | 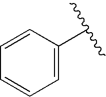 | 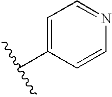 | H |
| 118 | H | CH | 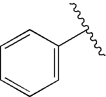 | 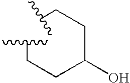 | |
| 119 | H | CH | 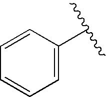 | 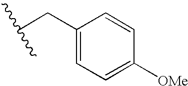 | H |
| 120 | H | CH | 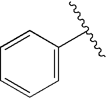 | 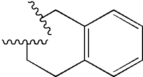 | |
| 121 | H | CH | 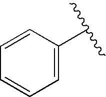 | 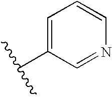 | H |
| 122 | H | CH | 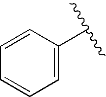 | 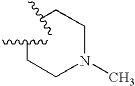 | |
| 123 | H | CH | 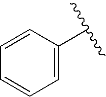 | 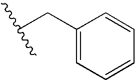 | H |
| 124 | H | CH | 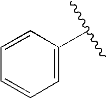 | 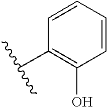 | H |
| 125 | H | CH | 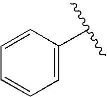 | 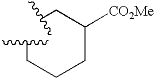 | |
| 126 | H | CH | 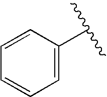 | 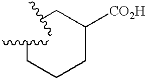 | |
| 127 | H | CH | 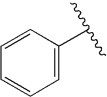 | 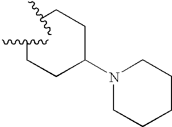 | |
| 128 | H | CH | 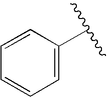 | 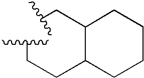 | |
| 129 | H | CH | 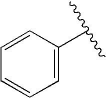 | 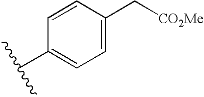 | H |
| 130 | H | CH | 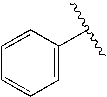 | 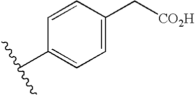 | H |

Table IV includes compounds of formula XXIII prepared by the procedures described above,

TABLE IV
Compounds of Formula XXIII

| | | | | (XXIII) |
|---|---|---|---|---|
| 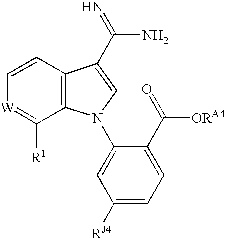 | | | | |
| Cmpd | | | | |
| No. | R¹ | W | R^{J4} | R^{A4} |
| 131 | H | CH | Br | —CH₃ |
| 132 | H | CH | Br | —CH₂CH₃ |
| 133 | H | CH | Br | H |
| 134 | H | CH | 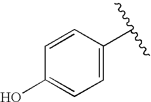 | —CH₃ |
| 135 | H | CH | 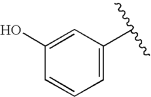 | —CH₃ |
| 136 | H | CH | 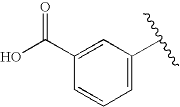 | —CH₃ |
| 137 | H | CH | 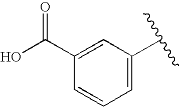 | H |
| 138 | H | CH | 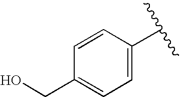 | —CH₃ |
| 139 | H | CH | 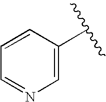 | —CH₃ |
| 140 | H | CH | 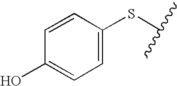 | —CH₂CH₃ |
| 141 | H | CH | H₂N— | —CH₃ |
| 142 | H | CH | 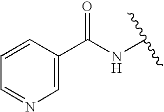 | —CH₃ |
| 143 | H | CH | 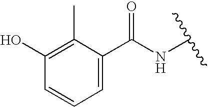 | —CH₃ |

Table V includes compounds of formula XXVI prepared by the procedures described above.

TABLE V
Compounds of Formula XXVI

| | |
|---|---|
| | (XXVI) |
| 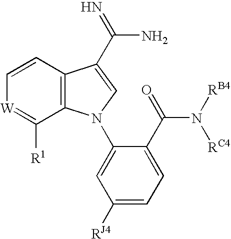 | |

| Cmpd | | | | | |
|---|---|---|---|---|---|
| No. | R¹ | W | R^{J4} | R^{B4} | R^{C4} |
| 144 | H | CH | Br | 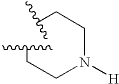 | |
| 145 | H | CH | Br | 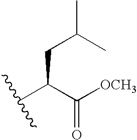 | H |
| 146 | H | CH | Br | 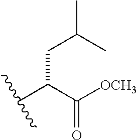 | H |
| 147 | H | CH | Br | 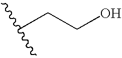 | H |
| 148 | H | CH | Br | —OCH₃ | —CH₃ |
| 149 | H | CH | Br | —CH₂CH(CH₃)₂ | H |
| 150 | H | CH | Br | 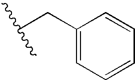 | H |

Table VI includes compounds of formula XXXII prepared by the procedures described above.

TABLE VI
Compounds of Formula XXXII

| | |
|---|---|
| | (XXXII) |
| 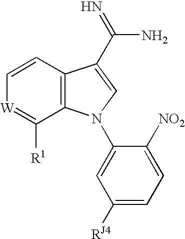 | |

| Compound | | | |
|---|---|---|---|
| No. | R¹ | W | R^{J4} |
| 151 | H | CH | H |
| 152 | H | CH | 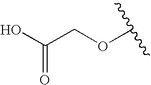 |
| 153 | H | CH | 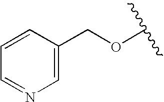 |
| 154 | H | CH | 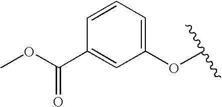 |
| 155 | H | CH | 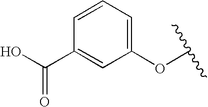 |
| 156 | H | CH | 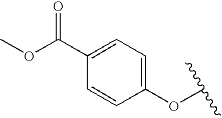 |
| 157 | H | CH | 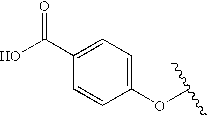 |
| 158 | 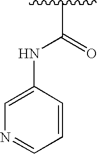 | CH | Cl |
| 159 | 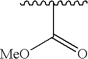 | CH | Cl |
| 160 | 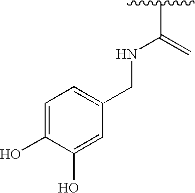 | CH | Cl |
| 161 | 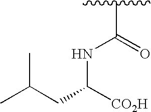 | CH | Cl |
| 162 | 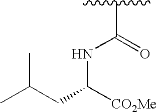 | CH | Cl |
| 163 | H | CH | Cl |
| 164 | H | CH | 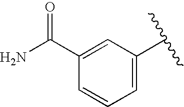 |
| 165 | H | CH | 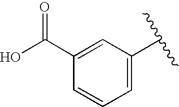 |
| 166 | H | CH | 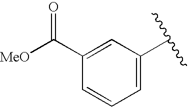 |
| 167 | H | CH | 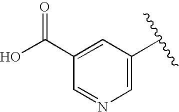 |
| 168 | H | CH | 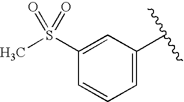 |
| 169 | H | CH | 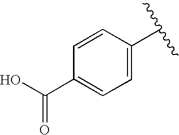 |
| 170 | 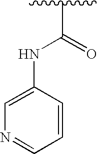 | CH | 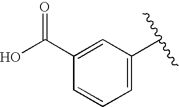 |
| 171 | 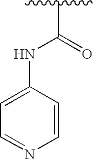 | CH | 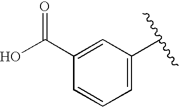 |
| 172 | 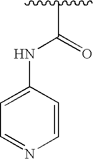 | CH | Cl |
| 173 | 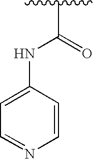 | CH | 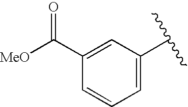 |

Table VII includes compounds of formula XXXII prepared by the procedures described above.

TABLE VII
Compounds of Formula XXXIII, XXXIV, and XXXV

| | | | | | (XXXIII) |
|---|---|---|---|---|---|
| 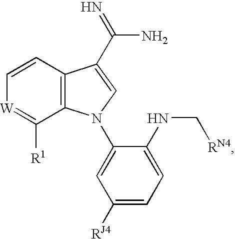 | | | | | |
| | | | | | (XXXIV) |
| 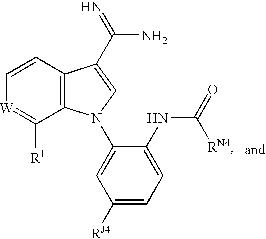 | | | | | |
| | | | | | (XXXV) |
| 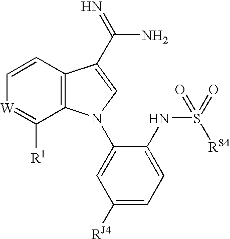 | | | | | |
| Cmpd | | | | | R^{N4} or |
| No. | R¹ | W | Formula | R^{J4} | R^{S4} |
| 174 | H | CH | XXXIII | 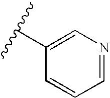 | H |
| 175 | H | CH | XXXIV | 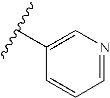 | CH₃ |
| 176 | H | CH | XXXV | 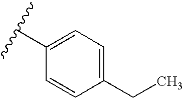 | CH₃ |

### Procedure X for the Preparation of Fluoroolefins of Formula LIV

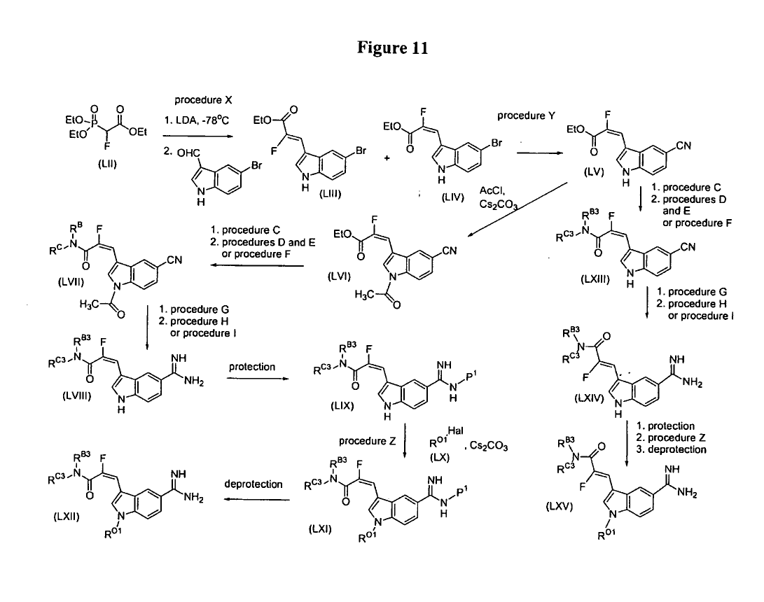

As shown in FIG. 11, the phosphonate of formula LII is treated with lithium diisopropylamide at −78° C. under an inert atmosphere, followed by reaction with commercially available 5-bromoindole-3-carboxaldehyde (Aldrich, Cat. No. 51, 874-3) to produce a mixture of fluoroolefins of formulas LIII (Z geometry) and LIV (E geometry), in a ratio of 1:7.5.

In one example, a 2.0 M LDA solution in THF (7.8 mL, 15.4 mmol) was added to THF (70 mL) at −25° C. To this LDA solution was added a solution of triethyl 2-fluoro-2-phosphonoacetate (3.53 g, 14.6 mmol) in THF (15 mL), followed by stirring at −25° C. for 45 min. The resulting carbanion solution was cooled to −78° C., followed by addition of 5-bromo-1H-indole-3-carbaldehyde (3.0 g, 13.4 mmol) in THF (28 mL). The reaction mixture was stirred for an additional 30 min at −78° C., and the cooling bath removed. The reaction mixture was stirred at room temperature for 3 h, then quenched with saturated NH₄Cl (30 mL) at 0° C. The separated aqueous layer is extracted with diethyl ether (80 mL×3). The combined organic layers is washed with brine, dried over Na₂SO₄, and concentrated in vacuo. The liquid residue was chromatographed on silica gel with AcOEt/Hexanes (0-20%) to give the desired (E)-fluoroolefin, 3-(5-cyano-1H-indol-3-yl)-2-fluoro-acrylic acid ethyl ester, in 88% yield.

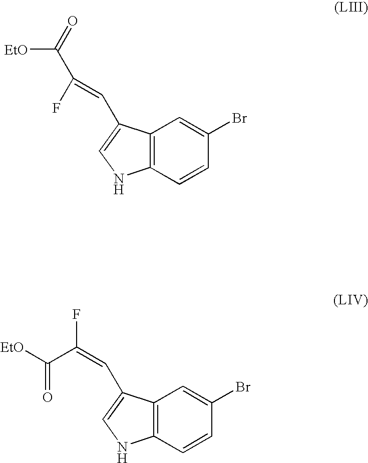

### General Procedure Y for Palladium-Mediated Cyanation

As shown in FIG. 11, using the procedure of Anderson et al. J. Org. Chem. 63: 8224-28, 1998, a mixture of NaCN, Cu(I), and palladium tetrakis-triphenylphosphine was treated with the compound of formula LIV to produce the compound of formula LV, which can be subsequently acylated with acetyl chloride using a mild base (such as, for example, cesium carbonate) to produce the compound of formula LVI as follows: an indole (1.0 mmol) is treated with acetyl chloride (1.5 mmol) or an anhydride (e.g. Boc anhydride) and DIEA (6.0 mmol) in DCM (14 mL) at room temperature. After 30 minutes, the solvent is removed. The residue is treated with NH₄Cl, and extracted with AcOEt. The organic layer is washed with brine (3×), dried over Na₂SO₄, filtered, and concentrated to afford the desired product. It was found that protection of the indole nitrogen with an electron withdrawing group retards the isomerization of the (E)-fluoroolefin to the (Z)-fluoroolefin during subsequent reactions.

Procedures C, D, E, G, I, and an amidine protection can be sequentially applied to the compound of formula LVI to produce a compound of formula LIX, where R^{B3} and R^{C3} are as previously defined, and P¹ is an amidine protecting group.

Alternatively, procedures C, D, E, G, and I can be sequentially applied to the compound of formula LV, resulting in isomerization of the olefin from the E geometry to the Z geometry during the Pinner conditions used in procedures G and I, to produce a compound of formula LXIV.

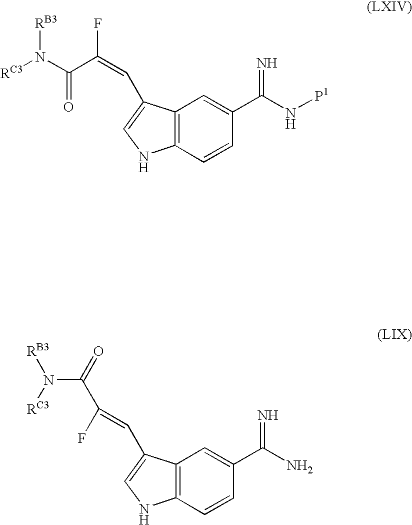

### General Indole Alkylation Procedure for the Preparation of Compounds of Formula LXII and LXV

As shown in FIG. 11, either the E isomer compound of formula LIX or the Z isomer compound of formula LXIV can be alkylated by treatment with an alkyl halide of formula LX, where R^{O1} is substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted heterocyclylalkyl, where the alkylene group is of one to six carbon atoms, to produce a compound of formula LXI or a compound of formula LXV, respectively. Compounds of formulas LXI can be deprotected to produce compounds of formula LXII and LXV, respectively, where R^{O1} is as previously defined.

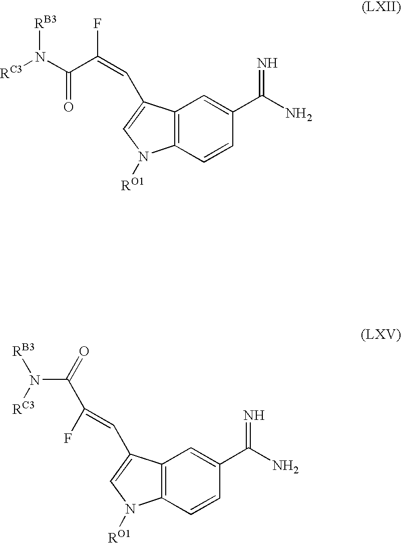

Table VIII includes compounds of formula LXII and LXV prepared by the procedures described above.

TABLE VIII
Compounds of Formula LXII and LXV

| Compounds No. | Formula | R^{O1} | R^{B3} | R^{C3} |
|---|---|---|---|---|
| 177 | LXII | H | H | H |
| 178 | LXV | H | H | 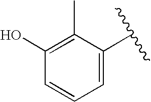 |
| 179 | LXII | H | H | 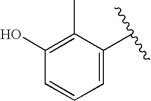 |
| 180 | LXII | H | H | 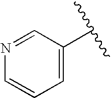 |
| 181 | LXII | H | H | 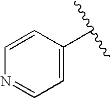 |

### Human Plasma Based Clotting Assay

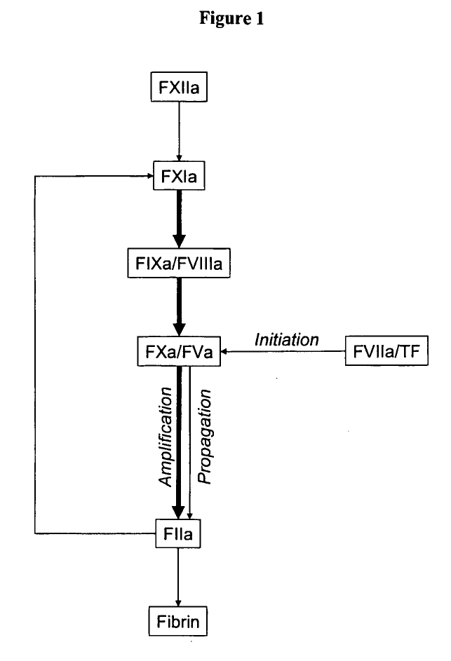

An activated partial thomboplastin time (aPTT) assay was used to measure the ability of compounds to inhibit the contact coagulation pathway. This pathway involves Factor XII, kallikrein, and Factor XI, which activates Factor IX and Factor VIII, leading to activation of Factor X and Factor V, and then activation of Factor II to form a blood clot (see FIG. 1). For this assay, CaCl₂ (30 mM) was placed in a large central reagent position of a Thromboscreen 400C instrument, allowing it to equilibrate to 37° C. Plasma (50 ul) and compounds of the invention were added at different concentrations to cuvettes. After incubation for two minutes, aPTT reagent (ALEXIN, Sigma) was added (50 ul) and incubated an additional three minutes. The cuvettes were transferred to a measuring position; prewarmed CaCl₂ reagent (50 ul) was added, and readings were then taken over a maximum of 300 seconds. A dose response curve was generated, and the concentration at which the clotting time was doubled (2×aPTT) was determined. Compounds which inhibit Factor XIa in the desired range desirably have an effect at less than 50 uM, more desirably at less than 10 uM.

A prothrombin time (PT) assay was also used to measure inhibition of coagulation. In this assay, the Factor XI dependent steps are bypassed. Hence, the assay measures inhibition of Factor VIIa, Factor Xa, and thrombin, but not FXI. This assay measures the ability of Factor VIIa to activate Factor X, which activates Factor II to form a blood clot. For this assay, the thromboplastin reagent (ThromboMax with Calcium, Sigma) was placed in a central reagent position in a Thromboscreen 400C instrument, and allowed to equilibrate to 37° C. Plasma (50 ul of plasma prewarmed for three minutes) and compounds of the invention (different concentrations) were added to cuvettes. The cuvettes were transferred to a measuring position. The prewarmed Thromboplastin reagent (100 ul) was then added, and readings were then taken over 300 seconds. A dose response curve was generated, and the concentration at which the clotting time was doubled (2×PT) was determined.

Table IX includes in vitro IC₅₀ data for selected compounds of the invention against factor XIa, factor Xa, and thrombin

TABLE IX
In vitro inhibition data

| Compound No. | IC₅₀ FXIa (μM) | IC₅₀ FXa (μM) | IC₅₀ thrombin (μM) |
|---|---|---|---|
| 8 | 0.62 | 170 | >200 |
| 34 | 1.39 | | |
| 47 | 1.77 | | >200 |
| 54 | 1.99 | | |
| 12 | 2.51 | | |
| 46 | 8.09 | | |
| 5 | 37.1 | 283 | >200 |
| 1 | 47.9 | 167 | >200 |
| 40 | 127 | | |
| 70 | 0.042 | >2 | >200 |
| 77 | 0.093 | 67.8 | >200 |
| 98 | 0.10 | | 3.78 |
| 95 | 0.87 | 82 | 4.2 |
| 84 | 1.47 | | |
| 64 | 1.96 | | |
| 58 | 3.23 | | |
| 91 | 3.81 | | |
| 101 | 6.27 | | |
| 111 | 13.7 | | |
| 106 | 22.4 | | |
| 108 | 44.6 | | |
| 151 | 4.15 | 145 | 224 |
| 131 | 1.36 | 35.1 | 60.2 |
| 139 | 3.80 | 9.65 | 73.1 |
| 135 | 1.62 | 15.2 | 43.7 |
| 142 | 11.3 | | |
| 154 | 3.00 | 54.7 | 20.2 |
| 150 | 280 | | |
| 144 | 81.7 | | |
| 148 | 134 | | |
| 158 | 1.24 | 80.8 | >200 |

### Administration of Compounds of the Invention for the Treatment or Prevention of Excess or Undesired Thrombosis

A compound of the invention may be used in any of the following clinical applications. For example, the compounds of the invention are useful for the treatment, stabilization, or prevention of a variety of medical disorders where anticoagulant therapy is indicated in the treatment or prevention of thrombotic conditions such as coronary artery and cerebro- and peripheral vascular disease. Indications include, but are not limited to, myocardial infarction, venous or arterial thrombosis, the formation of atherosclerotic plaques, coagulation syndromes, endarterectomy, including carotid endarterectomy, envascular injury including reocclusion and restenosis following angioplasty and coronary artery bypass surgery, thrombus formation after the application of blood vessel operative techniques, the introduction of artificial heart valves or on the recirculation of blood, cerebral infarction, cerebral thrombosis, transient ischemic attacks, stroke, cerebral embolism, pulmonary embolism, ischaemia, and angina, including unstable angina. In addition, pathologic thrombus formation often occurs in the venous vasculature of the lower extremities following knee, hip, and abdominal surgery (e.g., deep vein thrombosis, DVT). DVT further predisposes the patient to a higher risk of pulmonary thromboembolism. Disseminated intravascular coagulopathy (DIC), a systemic condition that commonly occurs in vascular systems during septic shock, certain viral infections, and cancer, is also indicated for treatment by compounds of the present invention. This condition is characterized by a rapid consumption of coagulation factors and their plasma inhibitors which can result in the formation of life-threatening clots throughout the microvasculature of several organ systems. Another application of Factor XIa inhibitors is the enhancement of fibrinolysis by tissue plasminogen activator.

In addition to their use in anticoagulant therapy, compounds of the invention are useful in the treatment and prevention of other diseases in which the generation of thrombin has been implicated as playing a physiologic role. For example, thrombin has been implicated in contributing to the morbidity and mortality of chronic and degenerative diseases such as cancer, arthritis, atherosclerosis, and Alzheimer's disease by its ability to regulate many different cell types through specific cleavage and activation of a cell surface thrombin receptor, mitogenic effects, diverse cellular functions such as cell proliferation, for example, abnormal proliferation of vascular cells resulting in restenosis or angiogenesis, release of PDGF, and DNA synthesis. Inhibition of Factor XIa effectively blocks thrombin generation and therefore neutralizes any physiologic effects of thrombin on various cell types. The representative indications discussed above include some, but not all, of the potential clinical situations amenable to treatment with a Factor XIa inhibitor.

Thus, one or more compounds of the invention can be used in the manufacture of medicaments for use in the production of an antithrombotic or anticoagulant. In one aspect, the invention features a method of treating, stabilizing, or preventing a disease, disorder, or condition associated with undesirable or excess thrombosis in a mammal (e.g., a human). This method involves administering a compound of the invention to the mammal in an amount sufficient to treat, stabilize, or prevent the disease, disorder, or condition. The compound may be administered to the mammal before, during, or after the occurrence of the condition.

In various embodiments, a compound that binds to Factor XI or Factor XIa decreases the activity of Factor XIa, the binding of a Factor XIa to another molecule (e.g., a substrate for Factor XIa), or the half-life of a Factor XI protein, as measured using standard methods (see, for example, Coligan, et al. Current Protocols in Protein Chemistry, Chapters 19 and 20, John Wiley & Sons, New York, 2000; Ausubel et al., Current Protocols in Molecular Biology, Chapter 9, John Wiley & Sons, New York, 2000). For example, the compound may competitively, noncompetitively, or uncompetitively inhibit the ability of Factor XI or Factor XIa to bind one or more of its endogenous substrates. The level of protein may be determined using standard Western, blot immunoassay, or immunohistochemical analysis (see, for example, Coligan, et al., supra; Ausubel et al., supra). Desirably, the compound decreases Factor XIa activity in an in vitro assay by at least 20, 40, 60, 80, 90 or 95%. In another desirable embodiment, the level of Factor XIa activity is at least 2, 3, 5, 10, 20, or 50-fold lower in the presence of the compoundin in an in vitro assay. In some embodiments, the compound is administered in a dose that is sufficient to reduce thrombosis but does not eliminate normal clotting resulting from external injuries or does not induce bleeding complications. In desirable embodiments, the in vivo half-life of an injected compound is less than 7, 6, 5, 4, 3, 2, 1, or 0.5 hours. In some embodiments, the in vivo half-life is contained within one of the following ranges: 4-6 hours, 2-4 hours, 30-120 minutes, or 30-60 minutes, inclusive. In desirable embodiments, the in vivo half-life of an oral compound is less than 24, 20, 16, 12, 8, or 4 hours. In some embodiments, the in vivo half-life is contained within one of the following ranges: 20-28 hours, 14-20 hours, 10-14 hours, 6-10 hours, 2-6 hours or 30-120 minutes, inclusive. In desirable embodiments, the compound has better inhibitory activity in an in vitro assay than benzamidine for Factor XIa at the same molar concentration, such as an IC₅₀ value of less than 100, 10, 1, 0.1, 0.01, or 0.001 μM.

With respect to the therapeutic methods of the invention, it is not intended that the administration of compounds to a mammal be limited to a particular mode of administration, dosage, or frequency of dosing; the present invention contemplates all modes of administration, including oral, intraperitoneal, intramuscular, intravenous, intraarticular, intralesional, subcutaneous, or any other route sufficient to provide a dose adequate to prevent or treat excess or undesired Factor XIa activity (e.g., excess or undesired clotting). One or more compounds may be administered to the mammal in a single dose or multiple doses. When multiple doses are administered, the doses may be separated from one another by, for example, several hours, one day, one week, one month, or one year. It is to be understood that, for any particular subject, specific dosage regimes should be adjusted over time according to the individual need and the professional judgment of the person administering or supervising the administration of the compositions. If desired, conventional treatments such as heparin may be used in combination with the compounds of the present invention. Exemplary mammals that can be treated using the methods of the invention include humans, primates such as monkeys, animals of veterinary interest (e.g., cows, sheep, goats, buffalos, and horses), and domestic pets (e.g., dogs and cats).

For clinical applications, compounds of the present invention may generally be administered, e.g., parenterally, intravenously, subcutaneously, intramuscularly, colonically, nasally, intraperitoneally, rectally, buccally, or orally. Compositions containing at least one compound of the invention that is suitable for use in human or veterinary medicine may be presented in forms permitting administration by a suitable route. These compositions may be prepared according to the customary methods, using one or more pharmaceutically acceptable adjuvants or excipients. The adjuvants comprise, inter alia, diluents, sterile aqueous media, and various non-toxic organic solvents. Acceptable carriers or diluents for therapeutic use are well known in the pharmaceutical field, and are described, for example, in Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988-1999, Marcel Dekker, New York. The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

The choice of vehicle and the content of active substance in the vehicle are generally determined in accordance with the solubility and chemical properties of the product, the particular mode of administration, and the provisions to be observed in pharmaceutical practice. For example, excipients such as lactose, sodium citrate, calcium carbonate, and dicalcium phosphate and disintegrating agents such as starch, alginic acids, and certain complex silicates combined with lubricants (e.g., magnesium stearate, sodium lauryl sulfate, and talc) may be used for preparing tablets. To prepare a capsule, it is advantageous to use lactose and high molecular weight polyethylene glycols. When aqueous suspensions are used, they may contain emulsifying agents which facilitate suspension. Diluents such as sucrose, ethanol, polyethylene glycol, propylene glycol, glycerol, chloroform, or mixtures thereof may also be used.

For parenteral administration, emulsions, suspensions, or solutions of the compositions of the invention in vegetable oil (e.g., sesame oil, groundnut oil, or olive oil), aqueous-organic solutions (e.g., water and propylene glycol), injectable organic esters (e.g., ethyl oleate), or sterile aqueous solutions of the pharmaceutically acceptable salts are used. The solutions of the salts of the compositions of the invention are especially useful for administration by intramuscular or subcutaneous injection. Aqueous solutions that include solutions of the salts in pure distilled water may be used for intravenous administration with the proviso that (i) their pH is adjusted suitably, (ii) they are appropriately buffered and rendered isotonic with a sufficient quantity of glucose or sodium chloride, and (iii) they are sterilized by heating, irradiation, or microfiltration. Suitable compositions containing the compounds of the invention may be dissolved or suspended in a suitable carrier for use in a nebulizer or a suspension or solution aerosol, or may be absorbed or adsorbed onto a suitable solid carrier for use in a dry powder inhaler. Solid compositions for rectal administration include suppositories formulated in accordance with known methods and containing at least one compound of formula I or II.

Dosage formulations of the compounds of this invention to be used for therapeutic administration must be sterile. Sterility is readily accomplished by filtration through sterile membranes (e.g., 0.2 micron membranes) or by other conventional methods. Formulations typically are stored in lyophilized form or as an aqueous solution. The pH of the compositions of this invention is typically between 3 and 11, more desirably between 5 and 9, and most desirably between 7 and 8, inclusive. While a desirable route of administration is by injection such as intravenously (bolus and/or infusion), other methods of administration may be used. For example, compositions may be administered subcutaneously, intramuscularly, colonically, rectally, nasally, or intrapertoneally in a variety of dosage forms such as suppositories, implanted pellets or small cylinders, aerosols, oral dosage formulations, and topical formulations such as ointments, drops, and dermal patches. The compounds of the invention are desirably incorporated into shaped articles such as implants, including but not limited to valves, stents, tubing, and prostheses, which may employ inert materials such as synthetic polymers or silicones, (e.g., Silastic, silicone rubber, or other commercially available polymers). Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the Factor XIa inhibitors of the invention may be coupled to a class of biodegradable polymers useful in achieving controlled release of a drug, for example polylactic acid, polyglycolic acid, copolymers of polylaclic and polyglycolic acid, polyepsilon caprolactone, polyhydroxy butyric acid, polyorthoesters, polyacetals, polydihydropyrans, polycyanoacrylates, and cross linked or amphipathic block copolymers of hydrogels.

The compounds of the invention may also be administered in the form of liposome delivery systems, such as small unilamellar vesicles, large unilamellar vesicles, and multilamellar vesicles. Liposomes can be formed from a variety of lipids, such as cholesterol, stearylamine, or phosphatidylcholines. The compounds of the invention may also be delivered using antibodies, antibody fragments, growth factors, hormones, or other targeting moieties to which the compound molecules are coupled (e.g., see Remington: The Science and Practice of Pharmacy, vide supra), including in vivo conjugation to blood components of a suitably modified compound of the formula I or II which possesses a metastable or reactive functional group as described above.

Dosage levels of active ingredients in the pharmaceutical compositions of the invention may be varied to obtain an amount of the active compound(s) that achieves the desired therapeutic response for a particular patient, composition, and mode of administration. The selected dosage level depends upon the activity of the particular compound, the route of administration, the severity of the condition being treated, and the condition and prior medical history of the patient being treated. For adults, the doses are generally from about 0.01 to about 100 mg/kg, desirably about 0.1 to about 1 mg/kg body weight per day by inhalation, from about 0.01 to about 100 mg/kg, desirably 0.1 to 70 mg/kg, more desirably 0.5 to 10 mg/kg body weight per day by oral administration, and from about 0.01 to about 50 mg/kg, desirably 0.1 to 1 mg/kg body weight per day by intravenous administration. Doses are determined for each particular case using standard methods in accordance with factors unique to the patient, including age, weight, general state of health, and other factors which can influence the efficacy of the compound(s) of the invention.

Administration of compositions of the invention may be as frequent as necessary to obtain the desired therapeutic effect. Some patients may respond rapidly to a higher or lower dose and may find much weaker maintenance doses adequate. Other patients, however, receive long-term treatments at the rate of 1 to 4 doses per day, in accordance with the physiological requirements of each patient. The active product may be administered, e.g., orally 1 to 4 times daily.

### OTHER EMBODIMENTS

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A compound of formula (I): 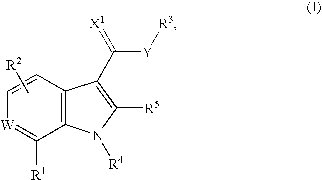
or pharmaceutically acceptable salt, pharmaceutically acceptable solvate, pharmaceutically active metabolite, or prodrug thereof, wherein
W is N or CR⁶, where R⁶ is H, halo, hydroxy, substituted or unsubstituted C₁₋₆ alkyl, substituted or unsubstituted C₂₋₆ alkenyl, substituted or unsubstituted C₁₋₆ alkoxy, substituted or unsubstituted C₂₋₁₂ alkoxyalkyl, where the alkylene group is of one to six carbon atoms, substituted or unsubstituted C₆ or C₁₀ aryl, substituted or unsubstituted C₆ or COO aryloxy, or —(CH₂)_{q}NR^{G6}R^{H6}, where q is an integer of from zero to two and each of R^{G6} and R^{H6} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N6}, where R^{N6} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms; (m) —CO₂R^{A6}, where R^{A6} is selected from the group consisting of hydrogen, substituted or unsubstituted C₁₋₄ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) —CONR^{B6}R^{C6}, where each of R^{B6} and R^{C6} is, independently, selected from the group consisting of hydrogen, substituted or unsubstituted C₁₋₄ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, and substituted or unsubstituted C₂₋₁₅ heterocyclylalkyl, where the alkylene group is of one to six carbon atoms, or R^{B6} taken together with R^{C6} and N forms a substituted or unsubstituted 5- or 6-membered ring, and (o) —S(O)₂R⁶, where R^{S1} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (p) a peptide chain of 1-4 natural or unnatural alpha-amino acid residues, where the peptide is linked via its C-terminal carboxy group to N, with the proviso that no two groups are bound to the nitrogen atom through a carbonyl group or a sulfonyl group;
X¹ is (H,H) or NR⁷, where R⁷ is H, C₁₋₆ alkyl, OH, NH₂, NO₂, CO₂R^{7a}, where R^{7a} is C₁₋₆ alkyl, or R⁷ taken together with R³ forms a 5- or 6-membered ring via a C₁ or C₂ linkage;
Y is NH or O, provided that when Y is O, X¹ is (H,H);
R¹ is a substituted or unsubstituted C₂₋₇ alkanoyl, substituted or unsubstituted C₁₋₆ alkyl, substituted or unsubstituted C₂₋₆ alkenyl, substituted or unsubstituted C₁₋₆ alkoxy, substituted or unsubstituted C₂₋₁₂ alkoxyalkyl, where the alkylene group is of one to six carbon atoms, substituted or unsubstituted C₁₋₆ alkylsulfinyl, substituted or unsubstituted C₂₋₁₂ alkylsulfinylalkyl, where the alkylene group is of one to six carbon atoms, substituted or unsubstituted C₁₋₆ alkylsulfonyl, substituted or unsubstituted C₆ or C₁₀ aryl, substituted or unsubstituted C₇₋₁₆ arylalkyl, where the alkylene group is of one to six carbon atoms, C₁ aminoalkyl, substituted or unsubstituted C₇ or C₁₁ aryloyl, substituted or unsubstituted C₃₋₈ cycloalkyl, substituted or unsubstituted C₄₋₁₄ cycloalkylalkyl, where the alkylene group is of one to six carbon atoms, substituted or unsubstituted C₁₋₉ heterocyclyl, substituted or unsubstituted C₁₋₉ (heterocyclyl)oxy, substituted or unsubstituted C₂₋₁₀ (heterocyclyl)oyl, substituted or unsubstituted C₁ thioalkoxy, substituted or unsubstituted C₂₋₁₂ thioalkoxyalkyl, where the alkylene group is of one to six carbon atoms, substituted or unsubstituted C₆ or C₁₀ aryloxy, substituted or unsubstituted C₃₋₈ cycloalkoxy, substituted or unsubstituted C₄₋₁₄ cycloalkylalkoxy, substituted or unsubstituted C₇₋₁₆ arylalkoxy, amidino, guanidino, ureido, —(CH₂)_{q}CO₂R^{A1}, where q is an integer of from zero to four and R^{A1} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}COR^{K1}, where q is an integer of from zero to four and R^{K1} is a peptide chain of 1-4 natural or unnatural alpha-amino acid residues, where the peptide is linked via its N-terminal amine to the CO group, —(CH₂)_{q}COR^{N1}, where q is an integer of from zero to four and R^{N1} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CONR^{B1}R^{C1}, where q is an integer of from zero to four and each of R^{B1} and R^{C1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B1} taken together with R^{C1} and N forms a substituted or unsubstituted 5- or 6-membered ring, —(CH₂)_{q}S(O)₂R^{D1}, where q is an integer of from zero to four and R^{D1} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide of 1-4 natural or unnatural alpha-amino acid residues, where the peptide is linked via its N-terminal amine to the S(O)₂ group, —(CH₂)_{q}S(O)₂NR^{E1}R^{F1}, where q is an integer of from zero to four and each of R^{E1} and R^{F1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E1} taken together with R^{F1} and N forms a substituted or unsubstituted 5- or 6-membered ring, —(CH₂)_{q}CR^{U1}═CR^{V1}CO₂R^{A1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{A1} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CR^{U1}═CR^{V1}COR^{K1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{K1} is a peptide chain of 1-4 natural or unnatural alpha-amino acid residues, where the peptide is linked via its N-terminal amine to the CO group, —(CH₂)_{q}CR^{U1}═CR^{V1}COR^{N1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{N1} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CR^{U1}═CR^{V1}CONR^{B1}R^{C1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₄alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and each of R^{B1} and R^{C1} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B1} taken together with R^{C1} and N forms a substituted or unsubstituted 5- or 6-membered ring, —(CH₂)_{q}CR^{U1}═CR^{V1}S(O)₂R^{D1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and R^{D1} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, —(CH₂)_{q}CR^{U1}═CR^{V1}S(O)₂NR^{E1}R^{F1}, where q is an integer of from zero to four, each of R^{U1} and R^{V1} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₇₋₁₆ aralkyl, (h) substituted or unsubstituted C₁₋₉ heterocyclyl, or (i) substituted or unsubstituted C₂₋₁₅ heterocyclylalkyl, and each of R^{E1} and R^{F1} is, independently, selected from the group consisting of (a) hydrogen, (b) substituted or unsubstituted C₁₋₄ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E1} taken together with R^{F1} and N forms a substituted or unsubstituted 5- or 6-membered ring, —(CH₂)_{q}NR^{G1}R^{H1}, where q is an integer of from zero to four and each of R^{G1} and R^{H1} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N1}, where R^{N1} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A1}, where R^{A1} is selected from the group consisting of hydrogen, substituted or unsubstituted C₁₋₄ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) —S(O)₂R^{S1}, where R^{S1} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (o) a peptide chain of 1-4 natural or unnatural alpha-amino acid residues, where the peptide is linked via its C-terminal carboxy group to N, with the proviso that no two groups are bound to the nitrogen atom through a carbonyl group or a sulfonyl group, —(CH₂)_{q}OR^{A1}, where q is an integer of from 0 to 4 and R^{A1} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or —(CH₂)_{q}NR^{b1}CONR^{E1}R^{F1}, where q is an integer of from 0 to 4, R^{b1} is hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and each of R^{E1} and R^{F1} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E1} taken together with R^{F1} and N forms a substituted or unsubstituted 5- or 6-membered ring;
R² is an H or a substituted or unsubstituted C₁₋₆ alkyl, substituted or unsubstituted C₂₋₄ alkenyl, hydroxyl, substituted or unsubstituted C₁₋₆ alkoxy, substituted or unsubstituted C₇₋₁₆ aralkoxy, trifluoromethyl, halo, amidino, N-hydroxyamidino, guanidino, —(CH₂)_{q}CO₂R², where q is an integer of from zero to two and R^{A2} is selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CONR^{B2}R^{C2}, where q is an integer of from zero to two and each of R^{B2} and R^{C2} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B2} taken together with R^{C2} and N forms a substituted or unsubstituted 5- or 6-membered ring, —(CH₂)_{q}S(O)₂R², where q is an integer of from zero to two and R^{D2} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}S(O)₂NR^{E2}R^{F2}, where q is an integer of from zero to two and each of R^{E2} and R^{F2} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E2} taken together with R^{F2} and N forms a substituted or unsubstituted 5- or 6-membered ring, or —(CH₂)_{q}NR^{G2}R^{H2}, where q is an integer of from zero to two and each of R^{G2} and R^{H2} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N2}, where R^{N2} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A2}, where R^{A2} is selected from the group consisting of hydrogen, substituted or unsubstituted C₁₋₆alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (n) —S(O)₂R^{S2}, where R^{S2} is selected from the group consisting of substituted or unsubstituted C₁₋₄ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, or R^{G2} taken together with R^{H2} and N forms a substituted or unsubstituted 5- or 6-membered ring, with the proviso that no two groups are bound to the nitrogen atom through a carbonyl group or a sulfonyl group;
R³ is H or C₁₋₆ alkyl, or when taken together with R⁵ or R⁷ forms a 5- or 6-membered ring via a C₁ or C₂ linkage;
R⁴ is H or a substituted or unsubstituted C₂₋₇ alkanoyl, substituted or unsubstituted C₁₋₆ alkyl, substituted or unsubstituted C₂₋₆ alkenyl, substituted or unsubstituted C₂₋₁₂ alkoxyalkyl, substituted or unsubstituted C₂₋₁₂ alkylsulfinylalkyl, substituted or unsubstituted C₁₋₆ alkylsulfonyl, substituted or unsubstituted C₆ or C₁₀ aryl, substituted or unsubstituted C₇₋₁₆ arylalkyl, substituted or unsubstituted C₁ aminoalkyl, substituted or unsubstituted C₇ or C₁₁ aryloyl, C₁₋₆ azidoalkyl, carboxaldehyde, carboxamide, C₃₋₈ cycloalkyl, C₄₋₁₄ cycloalkylalkyl, substituted or unsubstituted C₁₋₉heterocyclyl, C₂₋₁₅ heterocyclylalkyl, C₂₋₁₀ (heterocyclyl)oyl, hydroxy, C₁₋₆ hydroxyalkyl, N-protected aminoalkyl, C₂₋₁₂ thioalkoxyalkyl, C₁₋₄ perfluoroalkyl, substituted or unsubstituted C₆ or C₁₀ aryloxy, —(CH₂)_{q}CR^{U4}═CR^{V4}CO₂R^{A4} where q is an integer of from zero to four, each of R^{U4} and R^{V4} is, independently, (a) hydrogen, (b) substituted or unsubstituted C₁₁ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{A4} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CR^{U4}═CR^{V4}COR^{K4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{K4} is a peptide chain of 1-4 natural or unnatural alpha-amino acid residues, where the peptide is linked via its N-terminal amine to the CO group, —(CH₂)_{q}CR^{U4}═CR^{V4}COR^{N4}, where q is an integer of from zero to four, each of R^{U4} and R^{V4} is, independently, (a) hydrogen, (b) substituted or unsubstituted C₁₋₄ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{N4} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CR^{U4}═CR^{V4}CONR^{B4}R^{C4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉heterocyclyl, or (e) substituted or unsubstituted C₂₋₁₅ heterocyclylalkyl, and each of R^{B4} and R^{C4} is, independently, selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B4} taken together with R^{C4} and N forms a substituted or unsubstituted 5- or 6-membered ring, —(CH₂)_{q}CR^{U4}═CR^{V4}S(O)₂R^{D4}, where q is an integer of from zero to four, each of R^{U4} and R^{V4} is, independently, (a) hydrogen, (b) substituted or unsubstituted C₁₋₄alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and R^{D4} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, —(CH₂)_{q}CR^{U4}═CR^{V4} S(O)₂NR^{E4}R^{F4}, where q is an integer of from zero to four, each of R^{U4} and R^{V4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, and each of R^{E4} and R^{F4} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E4} taken together with R^{F4} and N forms a substituted or unsubstituted 5- or 6-membered ring, —(CH₂)_{q}(CR^{Y4}R^{Z4})ᵣ(CH₂)ₛCO₂R^{A4}, where each of q and s is, independently, an integer of from zero to four, and r is an integer of from zero to one, each of R^{Y4} and R^{Z4} is, independently, (a) hydrogen, (b) substituted or unsubstituted C₁₋₆alkyl, (c) substituted or unsubstituted C₆ or C₁₀ aryl, (d) substituted or unsubstituted C₁₋₉heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆ alkylsulfonyl, or (l) aminosulfonyl, and R^{A4} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or taken together with R^{Y4} or R^{Z4} forms an optionally substituted or unsubstituted 5- or 6-membered ring, —(CH₂)_{q}(CR^{Y4}R^{Z4})ᵣ(CH₂)ₛC(O)R^{K4}, where each of q and s is, independently, an integer of from zero to four, and r is an integer of from zero to one, each of R^{Y4} and R^{Z4} is, independently, (a) hydrogen, (b) substituted or unsubstituted C₁₋₆alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆ alkylsulfonyl, or (l) aminosulfonyl, and R^{K4} is a peptide chain of 1-4 natural or unnatural alpha-amino acid residues, where the peptide is linked via its N-terminal amine to the CO group, —(CH₂)_{q}(CR^{Y4}R^{Z4})ᵣ(CH₂)ₛC(O)R^{N4}, where each of q and s is, independently, an integer of from zero to four, and r is an integer of from zero to one, each of R^{Y4} and R^{Z4} is, independently, (a) hydrogen, (b) substituted or unsubstituted C₁₁ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₄₇ alkylsulfonyl, or (l) aminosulfonyl, and R^{N4} is selected from the group consisting of (a) substituted or unsubstituted C₁₋₄ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, or taken together with R^{Y4} or R^{Z4} forms an optionally substituted or unsubstituted 5- to 7-membered ring, —(CH₂)_{q}(CR^{Y4}R^{Z4})ᵣ(CH₂)₅CONR^{B4}R^{C4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₄ alkylsulfonyl, or (l) aminosulfonyl, and each of R^{B4} and R^{C4} is, independently, selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B4} taken together with R^{Y4} or R^{Z4} forms an optionally substituted or unsubstituted 5- or 6-membered ring, or R⁴ taken together with R^{C4} and N forms an optionally substituted 5- or 6-membered ring, —(CH₂)_{q}(CR^{Y4}R^{Z4})ᵣ(CH₂)ₛS(O)₂R^{D4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆₇ alkylsulfonyl, or (l) aminosulfonyl, and R^{D4} is selected from the group consisting of (a) hydroxyl, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, or taken together with R^{Y4} or R^{Z4} forms an optionally substituted or unsubstituted 5- to 7-membered ring, —(CH₂)_{q}(CR^{Y4}R^{Z4})ᵣ(CH₂)ₛS(O)₂NR^{E4}R^{F4} where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₆₇ alkylsulfonyl, or (l) aminosulfonyl, and each of R^{E4} and R^{F4} is independently selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E4} taken together with R^{Y4} or R^{Z4} forms an optionally substituted or unsubstituted 5- or 6-membered ring, or R^{E4} taken together with R^{F4} forms an optionally substituted 5- or 6-membered ring, —(CH₂)_{q}(CR^{Y4}R^{Z4})ᵣ(CH₂)ₛNR^{G4}R^{H4}, where each of q and s is, independently, an integer of from zero to four, and r is an integer of from zero to one, each of R^{Y4} and R^{Z4} is, independently, (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, (e) substituted or unsubstituted C₂₋₁₅ heterocyclylalkyl, (f) substituted or unsubstituted C₂₋₇ alkanoyl, (g) substituted or unsubstituted C₇ or C₁₁ aroyl, (h) substituted or unsubstituted C₃₋₁₀ heteroaroyl, (i) substituted or unsubstituted C₂₋₇ alkoxycarbonyl, (j) aminocarbonyl, (k) substituted or unsubstituted C₁₋₄ alkylsulfonyl, or (l) aminosulfonyl, and each of R^{G4} and R^{H4} is independently selected from the group consisting of (a) hydrogen, (b) an N-protecting group, (c) substituted or unsubstituted C₁₁ alkyl, (d) substituted or unsubstituted C₂₋₄ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N4}, where R^{N4} is selected from the group consisting of substituted or unsubstituted C₁₁ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A4}, where R^{A4} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂ heterocyclylalkyl, where the alkylene group is of one to six carbon atoms, (n) —S(O)₂R⁴, where R^{S4} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₅ heterocyclylalkyl, where the alkylene group is of one to six carbon atoms, with the proviso that no two groups are bound to the nitrogen atom through a carbonyl group or a sulfonyl group, or R^{G4} taken together with R^{Y4} or R^{Z4} forms an optionally substituted or unsubstituted 5- to 7-membered ring, or R^{G4} taken together with R^{H4} and N forms an optionally substituted 5- or 6-membered ring, or (o) —CONR^{E4}R^{F4}, and each of R^{E4} and R^{F4} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E4} taken together with R^{Y4} or R^{Z4} forms an optionally substituted or unsubstituted 5- to 7-membered ring, or R^{E4} taken together with R^{F4} and N forms a substituted or unsubstituted 5- or 6-membered ring; and
R⁵ is H or a substituted or unsubstituted C₁₋₆alkyl, C₂₋₆ alkenyl, C₁₋₆ alkoxy, carboxamide, C₃₋₈ cycloalkyl, hydroxy, nitro, nitrile, C₁₋₆ thioalkoxy, C₁₋₄ perfluoroalkyl, C₁₋₄ perfluoroalkoxy, —(CH₂)_{q}NR^{G5}R^{H5}, where q is zero to two and each of R^{G5} and R^{H5} is, independently, selected from the group consisting of (a) hydrogen, (b) an N-protecting group, (c) alkyl of one to six carbon atoms, (d) alkenyl of two to six carbon atoms, (e) alkynyl of two to six carbon atoms, and (f) cycloalkyl of three to eight carbon atoms, and (g) cycloalkylalkyl, where the cycloalkyl group is of three to eight carbon atoms.

2. The compound of claim 1, wherein R¹ is a substituted or unsubstituted C₂₋₇ alkanoyl, substituted or unsubstituted C₁₁ alkyl, substituted or unsubstituted C₂₋₄ alkenyl, substituted or unsubstituted C₇ or C₁₁ aryloyl, substituted or unsubstituted C₂₋₁₀ (heterocyclyl)oyl, or —(CH₂)_{q}CR^{U1}═CR^{V1}CONR^{B1}R^{C1}, where q is an integer of from zero to four, each of R^{U1} and R^{V1} is, independently, (a) hydrogen, (b) halogen, (c) hydroxyl, (d) substituted or unsubstituted C₁₋₄ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and each of R^{B1} and R^{C1} is, independently, selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{B1} taken together with R^{C1} and N forms a substituted or unsubstituted 5- or 6-membered ring.

3. The compound of claim 2, wherein R^{B1} is H and R^{C1} is 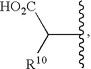
wherein
R¹⁰ is an substituted or unsubstituted C₁₋₆ alkyl, substituted or unsubstituted C₆ or C₁₀ aryl, substituted or unsubstituted C₇₋₁₆ aralkyl, substituted or unsubstituted C₁₋₉ heterocyclyl, or C₂₋₁₅ heterocyclylalkyl.

4. The compound of claim 1, wherein X¹ is NR⁶.

5. The compound of claim 1, wherein R⁴ is —CH₂C₂R^{A4}, —CH₂CONR^{B4}R^{C4}, —CH₂S(O)₂R^{D4}, —CH₂S(O)₂NR^{E4}R^{F4}, —CH₂C(O)R⁴, or —CH₂C(O)R^{N4}, wherein each of R^{A4}, R^{B4}, R^{C4}, R^{D4}, R^{E4}, and R^{F4} is as previously defined, and R^{K4} is a peptide chain of 1-4 natural or unnatural alpha-amino acid residues, where the peptide is linked via its N-terminal amine to the CO group.

6. The compound of claim 1, wherein R⁴ is —CR^{U4}═CHCONR^{B4}R^{C4}, wherein R^{U4} is (a) hydrogen, (b) substituted or unsubstituted C₁₋₆ alkyl, (c) substituted or unsubstituted C₆ or C₁₀ aryl, (d) substituted or unsubstituted C₁₋₉ heterocyclyl, or (e) substituted or unsubstituted C₂₋₁₅ heterocyclylalkyl, R^{B4} is H or substituted or unsubstituted C₁₋₆ alkyl, and R^{C4} is a substituted or unsubstituted C₁₋₄ alkyl, substituted or unsubstituted C₃₋₈ cycloalkyl, substituted or unsubstituted C₂₋₆ alkenyl, substituted or unsubstituted C₆ or C₁₀ aryl, substituted or unsubstituted C₇₋₁₆ arylalkyl, substituted or unsubstituted C₁₋₉ heterocyclyl, or substituted or unsubstituted C₂₋₁₅ heterocyclylalkyl.

7. The compound of claim 1, wherein R⁴ is a substituted or unsubstituted C₆ or C₁₀ aryl, substituted or unsubstituted C₇₋₁₆ arylalkyl, substituted or unsubstituted C₁₋₉heterocyclyl or substituted or unsubstituted C₂₋₁₅ heterocyclylalkyl.

8. The compound of claim 7, wherein R⁴ is selected from the group consisting of: 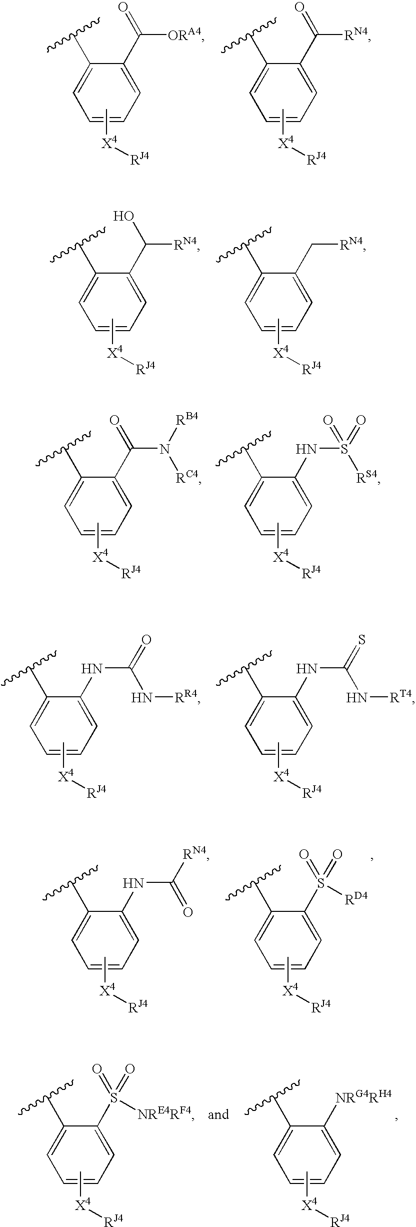
wherein
each of R^{A4}, R^{B4}, R^{C4}, R^{N4}, R^{R4}, R^{S4} and R^{T4} is, independently hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms;
X⁴ is O, S, NR¹⁴, or does not exist, wherein R¹⁴ is hydrogen or substituted or unsubstituted C₁₋₆ alkyl; and
R^{J4} is hydrogen, NO₂, SO₃H, CO₂H, substituted or unsubstituted C₁₋₆ alkyl, substituted or unsubstituted C₁₋₆ alkenyl, substituted or unsubstituted C₇₋₁₆ aralkyl, substituted or unsubstituted C₈₋₁₆ aralkenyl, substituted or unsubstituted C₂₋₁₅ heteroaralkyl, substituted or unsubstituted C₃₋₁₅ heteroaralkenyl, substituted or unsubstituted C₂₋₇ acyl, substituted or unsubstituted C₇₋₁₁ aroyl, substituted or unsubstituted C₃₋₁₀ heteroaroyl, substituted or unsubstituted C₂₋₇ alkoxycarbonyl, substituted or unsubstituted C₄₋₉ cycloalkoxycarbonyl, substituted or unsubstituted C₈₋₁₇ aralkoxycarbonyl, substituted or unsubstituted C₇ or C₁₁ aryloxycarbonyl, substituted or unsubstituted C₃₋₁₆ heteroaralkyloxycarbonyl, substituted or unsubstituted C₂₋₁₀ heterocyclyloxycarbonyl, aminocarbonyl, substituted or unsubstituted C₂₋₇ alkylaminocarbonyl, substituted or unsubstituted C₃₋₁₃ dialkylaminocarbonyl, substituted or unsubstituted C₄₋₉ cycloalkylaminocarbonyl, substituted or unsubstituted C₈₋₁₇ aralkylaminocarbonyl, substituted or unsubstituted C₇ or C₁₁ arylaminocarbonyl, substituted or unsubstituted C₃₋₁₀ heterocyclylaminocarbonyl, C₃₋₁₆heteroaralkylaminocarbonyl, substituted or unsubstituted C₂₋₇ alkylthiocarbonyl, substituted or unsubstituted C₄₋₉ cycloalkylthiocarbonyl, substituted or unsubstituted C₇₋₁₁ arylthiocarbonyl, substituted or unsubstituted C₈₋₁₇ aralkylthiocarbonyl, substituted or unsubstituted C₂₋₁₀ heterocyclylthiocarbonyl, substituted or unsubstituted C₃₋₁₆ heteroaralkylthiocarbonyl, substituted or unsubstituted C₁₋₆ alkylsulfonyl, substituted or unsubstituted C₃₋₈ cycloalkylsulfonyl, substituted or unsubstituted C₇₋₁₆ aralkylsulfonyl, substituted or unsubstituted C₆ or C₁₀ arylsulfonyl, substituted or unsubstituted C₂₋₉ heterocyclylsulfonyl, or a substituted or unsubstituted C₂₋₁₅ heteroaralkylsulfonyl, with the proviso that R¹⁴ is not SO₃H or CO₂H when X⁴ is O or S.

9. The compound of claim 7, wherein R¹ is —(CH₂)_{q}COR^{K1}, wherein q is an integer of from zero to four and R^{K1} is a peptide chain of 1-4 natural or unnatural alpha-amino acid residues, where the peptide is linked via its N-terminal amine to the CO group.

10. The compound of claim 2, wherein W is CH; X is NR⁶; each of R² and R³ is H; and R⁴ is —CH₂CO₂R^{A4}, —CH₂CONR^{B4}R^{C4}, —CH₂S(O)₂R^{D4}, —CH₂S(O)₂NR^{E4}R^{F4}, or —CH₂C(O)R^{A4}, wherein each of R^{A4}, R^{B4}, R^{C4}, R^{D4}, R^{E4}, and R^{F4} is as previously defined.

11. The compound of claim 2, wherein W is CH; X is NR⁶; each of R² and R³ is H; and R⁴ is —(CR^{Y4}R^{Z4})CH₂CO₂R^{A4}, —(CR^{Y4}R^{Z4})CH₂CONR^{B4}R^{C4}, —(CR^{Y4}R^{Z4})CH₂S(O)₂R^{D4}, —(CR^{Y4}R^{Z4})CH₂S(O)₂NR^{E4}R^{F4}, or —(CR^{Y4}R^{Z4})CH₂C(O)R^{K4}, wherein each of R^{A4}, R^{B4}, R^{C4}, R^{D4}, R^{E4}, R^{F4}, R^{K4}, R^{Y4} and R^{Z4} is as previously defined.

12. Use of a compound of claim 1, or a pharmaceutically acceptable salt or prodrug thereof, in the preparation of a medicament for the treatment of a thromboembolic disorder.

13. The use of claim 12, wherein said thromboembolic disorder is selected from the group consisting of arterial cardiovascular thromboembolic disorders, venous cardiovascular thromboembolic disorders, and thromboembolic disorders in the chambers of the heart.

14. The use of claim 12, wherein said thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

15. A compound of formula (I): 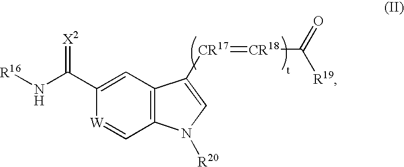
or a pharmaceutically acceptable salt, pharmaceutically acceptable solvate, pharmaceutically active metabolite, or prodrug thereof, wherein
t is 0 or 1;
W is N or CR²¹, wherein R²¹ is H, halo, amino, hydroxy, substituted or unsubstituted C₁₋₆ alkoxy, substituted or unsubstituted C₁₋₆ alkyl, or forms a 5- or 6-membered ring with R²² via a C₁ or C₂ linkage;
X² is (H,H) or NR²², wherein R²² is H, C₁₋₆ alkyl, OH, NH₂, NO₂, CO₂R^{22a}, wherein R^{22a} is C₁₋₆ alkyl, or R²² taken together with R¹⁶ or R²¹ forms a 5- or 6-membered ring via a C₁ or C₂ linkage;
R¹⁶ is H, substituted or unsubstituted C₁₋₄ alkyl, or when taken together with R²² forms a 5- or 6-membered ring via a C₁ or C₂ linkage;
each of R¹⁷ and R¹⁸ is, independently H, halo, or C₁₋₆ alkyl;
R¹⁹ is C₁₋₆ alkyl, C₃₋₈ cycloalkyl, OR²³, or NR²³R²⁴, wherein each of R²³ or R²⁴ is, independently, H, substituted or unsubstituted C₁₋₆ alkyl, C₃₋₈ cycloalkyl, or C₂₋₆ alkenyl;
W is N or CR²¹, wherein R²¹ is H, halo, amino, hydroxy, substituted or unsubstituted C₁₋₆ alkoxy, substituted or unsubstituted C₁₋₆ alkyl, or forms a 5- or 6-membered ring with R²² via a C₁ or C₂ linkage;
X² is (H,H) or NR²², wherein R²² is H, Clot alkyl, OH, NH₂, NO₂, CO₂R^{22a}, wherein R^{22a} is C₁₋₆ alkyl, or R²² taken together with R¹⁶ or R²¹ forms a 5- or 6-membered ring via a C₁ or C₂ linkage;
R¹⁶ is H, substituted or unsubstituted C₁₋₆ alkyl, or when taken together with R²² forms a 5- or 6-membered ring via a C₁ or C₂ linkage;
each of R¹⁷ and R¹⁸ is, independently H, halo, or C₁₋₆alkyl;
R¹⁹ is C₁₋₆ alkyl, C₃₋₈ cycloalkyl, OR²³, or NR²³R²⁴, wherein each of R²³ or R²⁴ is, independently, H, substituted or unsubstituted C₁₋₆ alkyl, C₃₋₈ cycloalkyl, or C₂₋₆ alkenyl;
R²⁰ is a substituted or unsubstituted C₂₋₇ alkanoyl, substituted or unsubstituted C₁₋₆ alkyl, substituted or unsubstituted C₂₋₆ alkenyl, substituted or unsubstituted C₁₋₆ alkoxy, substituted or unsubstituted C₂₋₁₂ alkoxyalkyl, where the alkylene group is of one to six carbon atoms, substituted or unsubstituted C₁₋₄ alkylsulfinyl, substituted or unsubstituted C₂₋₁₂ alkylsulfinylalkyl, where the alkylene group is of one to six carbon atoms, substituted or unsubstituted C₁₋₄ alkylsulfonyl, substituted or unsubstituted C₆ or C₁₀ aryl, substituted or unsubstituted C₇₋₁₆ arylalkyl, where the alkylene group is of one to six carbon atoms, C₁₋₆ aminoalkyl, substituted or unsubstituted C₇ or C₁₁ aryloyl, substituted or unsubstituted C₃₋₈ cycloalkyl, substituted or unsubstituted C₄₋₁₄ cycloalkylalkyl, where the alkylene group is of one to six carbon atoms, substituted or unsubstituted C₁₋₉ heterocyclyl, C₂₋₁₅ heterocyclylalkyl, substituted or unsubstituted C₁₋₉ (heterocyclyl)oxy, substituted or unsubstituted C₂₋₁₀ (heterocyclyl)oyl, substituted or unsubstituted C₁₋₆ thioalkoxy, substituted or unsubstituted C₂₋₁₂ thioalkoxyalkyl, where the alkylene group is of one to six carbon atoms, substituted or unsubstituted C₆ or C₁₀ aryloxy, substituted or unsubstituted C₃₋₈ cycloalkoxy, substituted or unsubstituted C₄₋₁₄ cycloalkylalkoxy, substituted or unsubstituted C₇₋₁₆ arylalkoxy, amidino, guanidino, ureido, —(CH₂)_{q}CO₂R^{A20}, wherein q is an integer of from zero to four and R^{A20} is selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}COR^{K20}, wherein q is an integer of from zero to four and R^{K20} is a peptide chain of 1-4 natural or unnatural alpha-amino acid residues, where the peptide is linked via its N-terminal amine to the CO group, —(CH₂)_{q}COR^{N20}, wherein q is an integer of from zero to four and R^{N20} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CONR^{B20}R^{C20}, wherein q is an integer of from zero to four and each of R^{B20} and R^{C20} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅heterocyclylalkyl, where the alkylene group is of one to six carbon atoms, or R^{B20} taken together with R^{C20} and N forms a substituted or unsubstituted 5- or 6-membered ring, —(CH₂)_{q}S(O)₂R^{D20}, wherein q is an integer of from zero to four and R^{D20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide of 1-4 natural or unnatural alpha-amino acid residues, where the peptide is linked via its N-terminal amine to the S(O)₂ group, —(CH₂)_{q}S(O)₂NR^{E20}R^{F20}, wherein q is an integer of from zero to four and each of R^{E20} and R^{F20} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, or R^{E20} taken together with R^{F20} and N forms a substituted or unsubstituted 5- or 6-membered ring, —(CH₂)_{q}CR^{U20}═CR^{V20}CO₂R^{A20}, wherein q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and R^{A20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, —(CH₂)_{q}CR^{U20}═CR^{V20}COR^{K20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₄ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and R^{K20} is a peptide chain of 1-4 natural or unnatural alpha-amino acid residues, where the peptide is linked via its N-terminal amine to the CO group, —(CH₂)_{q}CR^{U20}═CR^{V20}COR^{N20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and R^{N20} is selected from the group consisting of (a) substituted or unsubstituted C₁₋₄ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms, —(CH₂)_{q}CR^{U2}═CR^{V20}CONR^{B20}R^{C20} where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and each of R^{B20} and R^{C20} is, independently, selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅heterocyclylalkyl, where the alkylene group is of one to six carbon atoms, or R^{B20} taken together with R^{C20} and N forms a substituted or unsubstituted 5- or 6-membered ring, —(CH₂)_{q}CR^{U20}═CR^{V20}S(O)₂R^{D20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₄ alkoxy, (f) substituted or unsubstituted C₆ or C₁₀ aryl, (g) substituted or unsubstituted C₁₋₉heterocyclyl, or (h) substituted or unsubstituted C₂₋₁₅ heterocyclylalkyl, and R^{D20} is selected from the group consisting of (a) hydrogen, (b) substituted or unsubstituted C₁₋₄ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, (f) substituted or unsubstituted C₂₋₁₅ heterocyclylalkyl, where the alkylene group is of one to six carbon atoms, and (g) a peptide chain of 1-4 natural or unnatural alpha-amino acid residues, where the peptide is linked via its N-terminal amine to the S(O)₂ group, —(CH₂)_{q}CR^{U2}O═CR^{V2} S(O)₂NR^{E20}R^{F20}, where q is an integer of from zero to four, each of R^{U20} and R^{V20} is, independently, (a) hydrogen, (b) halogen, (c) hydroxyl, (d) substituted or unsubstituted C₁₋₆ alkyl, (e) substituted or unsubstituted C₁₋₆ alkoxy, (f) substituted or unsubstituted C₆ or C₁₀ aryl, (g) substituted or unsubstituted C₁₋₉ heterocyclyl, or (h) substituted or unsubstituted C₂₋₁₅ heterocyclylalkyl, and each of R^{E20} and R^{F20} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E20} taken together with R^{F20} and N forms a substituted or unsubstituted 5- or 6-membered ring, —(CH₂)_{q}NR^{G20}R^{H20}, where q is an integer of from zero to four and each of R^{G20} and R^{H20} is, independently, selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) C₃₋₈ cycloalkyl, (g) cycloalkylalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ arylalkyl, where the alkylene group is of one to six carbon atoms, (j) substituted or unsubstituted C₁₋₉ heterocyclyl, (k) substituted or unsubstituted C₂₋₁₅ heterocyclylalkyl, where the alkylene group is of one to six carbon atoms, (l) —COR^{N20}, where R^{N20} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (m) —CO₂R^{A20}, where R^{A20} is selected from the group consisting of hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, (n) (CH₂)_{q}OR^{A20}, where q is an integer of from 0 to 4 and R^{A20} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms, (o) —S(O)₂R^{S20}, where R^{S20} is selected from the group consisting of substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and (p) a peptide chain of 1-4 natural or unnatural alpha-amino acid residues, where the peptide is linked via its C-terminal carboxy group to N, with the proviso that no two groups are bound to the nitrogen atom through a carbonyl group or a sulfonyl group, or —NR^{b20}CONR^{E20}R^{F20}, where R^{b20} is hydrogen, substituted or unsubstituted C₁₋₆ alkyl, substituted or unsubstituted C₆ or C₁₀ aryl, substituted or unsubstituted C₇₋₁₆ arylalkyl, where the alkylene group is of one to six carbon atoms, substituted or unsubstituted C₁₋₉ heterocyclyl, or substituted or unsubstituted C₂₋₁₅ heterocyclylalkyl, where the alkylene group is of one to six carbon atoms, and each of R^{E20} and R^{F20} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted cycloalkylalkyl, where the cycloalkyl group is of three to eight carbon atoms, and the alkylene group is of one to ten carbon atoms (e) substituted or unsubstituted C₆ or C₁₀ aryl, (f) substituted or unsubstituted C₇₋₁₆ arylalkyl, where the alkylene group is of one to six carbon atoms, (g) substituted or unsubstituted C₁₋₉ heterocyclyl, and (h) substituted or unsubstituted C₂₋₁₅ heterocyclylalkyl, where the alkylene group is of one to six carbon atoms, or R^{E20} taken together with R^{F20} and N forms a substituted or unsubstituted 5- or 6-membered ring.

16. The compound of claim 15, wherein R¹⁷ is hydrogen and R¹⁸ is F.

17. The compound of claim 15, wherein X² is NH and R¹⁶ is H.

18. Use of a compound of claim 15, or a pharmaceutically acceptable salt or prodrug thereof, in the preparation of a medicament for the treatment of a thromboembolic disorder.

19. The use of claim 18, wherein the thromboembolic disorder is selected from the group consisting of arterial cardiovascular thromboembolic disorders, venous cardiovascular thromboembolic disorders, and thromboembolic disorders in the chambers of the heart.

20. The use of claim 18, wherein the thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.
